(19) United States
(12) Patent Application Publication
(10) Pub. No.: US 2025/0391519 A1
(43) Pub. Date: Dec. 25, 2025

(54) CHEMICAL SYNTHESIS PLATFORM

(71) Applicant: THE UNIVERSITY COURT OF THE UNIVERSITY OF GLASGOW, Glasgow (GB)

(72) Inventor: Leroy CRONIN, Glasgow (GB)

(21) Appl. No.: 18/879,555

(22) PCT Filed: Jun. 28, 2023
(86) PCT No.: PCT/EP2023/067674

§ 371 Date: Dec. 27, 2024

(30) Foreign Application Priority Data
Jun. 28, 2022 (GB) 2209476.7

(51) Int. Cl. G16C 20/90; G16C 20/10

(52) U.S. Cl. CPC G16C 20/90; G16C 20/10

(57) ABSTRACT

The invention relates to a method for generating a database for chemical syntheses and a method for performing a chemical synthesis using a database. The method for generating a database comprises generating a series of instruction sets for a series of chemical syntheses from the literature, wherein each instruction set is a machine readable and executable universal language for a chemical synthesiser; assembling the series of instructions sets within a database, and making the instruction sets available for access and autonomous execution by a chemical synthesiser. The method for performing a chemical synthesis comprises accessing an instruction set from a database, providing the instruction set to a chemical synthesiser, and autonomously executing the instruction set on the chemical synthesiser thereby to perform the chemical synthesis. The invention makes use of a standardised and step-focused syntax to describe the chemical syntheses, and a universal synthesis automation platform.

### RELATED APPLICATIONS

The present application claims priority to, and the benefit of, GB 2209476.7 filed on 28 Jun. 2022, the contents of which are hereby incorporated by reference in their entirety.

### FIELD OF THE INVENTION

The present invention provides methods for generating a chemical synthesis database, and the use of the database, with verification, for automatic chemical syntheses on a chemical synthesiser.

### BACKGROUND

To replicate a known chemical reaction the protocol must be obtained from the literature, or a database, so that it can be run manually in the laboratory. (1) However, not all the literature or database entries can be easily reproduced. (2) This is a barrier, not only to the synthesis of new molecules, but to accumulation of high quality data for machine learning, (3-6) and is exacerbated by the fact there is also no open standard for coding the procedures, for example for automated chemical synthesisers, or a way to report and correct failed experiments. (7, 8)

Chemical synthesis currently requires intensive, highly skilled labour (17) and a typical synthesis can require multiple complex unit operations that are difficult to explicitly encode. This is because the tacit knowledge required is often context dependent resulting in ambiguities in the published literature, and hence limits reproducibility, automation or data mining. (18) These limits have been overcome in some specific areas such as oligopeptide (19), oligosaccharide (20), and oligonucleotide (21) chemistry, and more recently much progress has been made in automating chemistry processes. (22-30) However most automated synthetic chemistry platforms remain task specific or represent islands of automation (10) in an otherwise manual workflow, but even these have bespoke instruction sets with no simple semantic link among them or to the literature. To fully exploit the potential of automation in chemical synthesis and ensure reproducibility of procedures, progress is needed.

The present inventor has previously shown how literature methods in natural language may be converted into a machine-readable instruction set: see WO 2021/219999. This instruction set may then be used to generate a hardware index, which is the required reactionware for the performance of a synthetic procedure.

Similarly, the present inventor has also shown how a method of synthesis for a target product can be translated into digital model of that method: see WO 2019/137954. This digital model comprises a digital description of the chemical and/or physical steps within the method of synthesis. From this, a digital reactionware may be developed, which provides a digital reaction module for each step in the process sequence, and the digital modules are digitally interconnected for the digital production of a target product. From this digital reactionware the physical reactionware is generated, with the physical reactionware has a module for each step in the process sequence.

### SUMMARY OF THE INVENTION

In a general aspect the present invention provides a method for generating a database for chemical syntheses, where the database holds a plurality of instructions sets for a chemical synthesiser, where each instruction set is a machine code for the execution of a chemical synthesis. The instructions set is a chemical description language that is fully described and universal. The database is accessible and the instruction sets are available for transfer to a chemical synthesiser on demand. The database may hold multiple instruction sets for a single chemical synthesis for version control, validation, collaboration and data mining.

The invention also provides for the use of the database for automatic chemical synthesis, where a chemical synthesiser accesses an instruction set in the database, and performs a chemical synthesis and purification according to the instruction set.

Also provided is a method for generating an instruction set for use on a chemical synthesiser. Here a chemical synthesis reported in the literature is translated to an instruction set, and a chemical synthesis is performed by a chemical synthesiser according to the instruction set. The chemical synthesis is assessed and where the performance of the synthesis is regarded as having a satisfactory performance, the instructions set may be uploaded to a database, where it may be made available for access.

A chemical synthesis may be regarded as satisfactory where it gives the target product in a target yield and/or purity. Here, the target yield and/or purity may be that reported in the literature for the synthesis. Alternatively, it may be a target that is set by the operator.

Where a chemical synthesis is not satisfactory, the instruction set may be revised and a further chemical synthesis performed to test where the revision gives a satisfactory result. The instruction set may be revised as needed until the desired target is achieved (or otherwise where the target is deemed inaccessible).

The running of an instruction set on a chemical synthesiser may be referred to as chenputation, and is the reliable conversion of code and reagents into a desired chemical product.

The instruction set, which may be referred to as a chemical description language, or _{X}DL, is a universal language, which is readable and executable by different chemical synthesisers.

Accordingly, the instruction set is an apparatus agnostic description of chemical operations, and a chemical synthesiser may interpret the instruction set to execute a synthesis of the target product on the chemical synthesiser.

The instruction set, or the chemical description language, is a fully described instruction set. Thus, the set provides the full details needed for the chemical synthesiser to undertake a chemical synthesis, and to access the desired chemical product, and, advantageously, to at least partially purify the desired product from other components of a reaction mixture.

In the first aspect of the invention, there is provided a method for generating a database for chemical syntheses, the method comprising the steps of:
(i) generating a series of instruction sets for a series of chemical syntheses from the literature, wherein each instruction set is a machine readable and executable language for a chemical synthesiser; and
(ii) assembling the series of instructions sets within a database, and making the instruction sets available for access and autonomous execution by a chemical synthesiser.

The database may be organised and searchable. Each instruction set is optionally provided with analytical data for the synthesis, including for the target product.

An instruction set may be generated together with analytical data for the synthesis, and wherein the instruction set is made available for access together with the analytical data for access by a chemical synthesiser.

An instruction set if made available for access together with information for access by a chemical synthesiser, which information may be selected from:
(i) a user ranking of the instruction set;
(ii) the identity of the originator of the instruction set;
(iii) the validation status of an instruction set, and where validated optionally the identity of the validator of the instruction set;
(iv) the access number for the instruction set, which access number is the number of times that instructions set has been viewed or the number of times the instruction set has been provided to a chemical synthesiser for execution, optionally together with a relative ranking of the instructions set amongst a group of instruction sets available from the database; and
(v) variant instruction sets for the chemical synthesis, where a variant instructions set is one in which at least one operation within the instruction set is modified over the instruction set, where a variant instruction set may give rise a reaction outcome for the chemical synthesis that differs from the reaction outcome for the chemical synthesis executed under the instruction set.

Instruction sets may be made available only to authorised chemical synthesiser, such as a paid subscriber to the database.

The literature is typically the chemical literature, including journal articles, that is written in natural language. The method of the invention translates these chemical syntheses into a format that is universal to chemical synthesisers in that the instruction set is executable regardless of the precise reactionware held by the chemical synthesiser.

The conversion of a literature process in a natural language to an instruction set for a chemical synthesiser may follow the methods developed by the present inventor in WO 2021/219999.

The database holds multiple instruction sets, and this database may provide a persistent, trusted and reliable store of experimental procedures for execution by chemical synthesisers.

In another aspect of the invention there is provided a method for performing a chemical synthesis, the method comprising the steps of:
(i) accessing an instruction set from a database, and providing the instruction set to a chemical synthesiser, where the instruction set is a machine readable and executable language for a chemical synthesiser; and
(ii) autonomously executing the instruction set on the chemical synthesiser thereby to perform the chemical synthesis.

The result of the chemical synthesis may be analysed, and is optionally reported to the database.

The instruction set may be provided to the chemical synthesiser together with analytical data, wherein the analytical data is compared with analytical data that is generated from the chemical synthesis.

The method may further comprise the step of (iii) modifying an operation within the instruction set, and subsequently autonomously executing the modified instruction set on the chemical synthesiser thereby to perform the chemical synthesis, and analytical data that is generated from the chemical synthesis executed under the modified instruction set is compared with the analytical data provided with the instructions.

The chemical synthesiser has the reactionware that is commonplace for the performance of chemical reactions. Thus, the chemical synthesiser may include modules selected from the group consisting of, and preferably including all of, a reactor, a separator, an evaporator and a purification system. The chemical synthesiser may include a chromatographic system within the purification system for column chromatography.

The chemical synthesiser is suitably programmed to create an execution schedule for the chemical synthesis which arises from the chemical processing unit of the synthesiser developing the instruction set with knowledge of the available physical components of the chemical synthesiser, notably the reactionware that is available to that specific synthesiser.

The development of graphs as a representation of the abstract layer of a reactionware, and the subsequent implementation of the graph in the physical layer for the execution of a chemical synthesis on an automated platform is described by the present inventor in WO 2019/137954.

The chemical synthesiser may be provided with the materials, such as reagents, solvents and catalysts, necessary for the performance of the chemical synthesis.

An instruction set may be provided together with analytical information for the synthesis, such as characterising information for the product of the synthesis. The method may then comprise a comparison of the analytical information of the chemical synthesis against that provided with the instruction set. The results of the chemical synthesis may be reported to the database, regardless of whether analytical data is the same or different to that analytical data provided with the instruction set.

Where a chemical synthesis is performed and the analytical results correspond to those provided with the instruction set, then the performance of the chemical synthesis may be regarded as having validated the instruction set. In this way, the reliability of the instruction set is established.

Optionally, after an instruction set is used and a chemical synthesis is performed, the instruction set may be altered in at least one aspect of its synthesis script, and the altered instruction set may then be executed on the chemical synthesiser thereby to perform the chemical synthesis. The analytical data for that synthesis may then be compared against the original instructions set to assess the impact of the change on the reaction result. In such a way the instructions set may be optimised for the chemical synthesis, or alternatively the conserved parts of the instructions set may be recognised and preserved for later iterations of the instruction set.

These and other aspects and embodiments of the invention are described in further detail below.

### SUMMARY OF THE FIGURES

The present invention is described with reference to the figures listed below.

FIG. 1—Schematic of _{X}DL protocol optimization. Chemical reactions can be captured by the Chemical Description Language (_{X}DL) which represents synthetic steps as sequences of physical processes such as Add, Dissolve, Evaporate etc. The initially established _{X}DL protocol is then executed on a chemical synthesiser and the purity and yield of the product are determined. The _{X}DL protocol can be improved until the process meets the expectation of product purity and yield. At that stage the protocol can be added to the database as validated, backed up by the full characterisation of the target product and the process development history.

FIG. 2—The ChemPU that drives the chemical synthesiser can be regarded as a chemical state machine. It processes the physical input (i.e., reagents, solvents) based on the digital input (i.e. the synthesis script). Each unit operation defines a route for the system to progress from the current state to the next state. The state of the system is always well-defined, and every detail of the synthesis is known and can be documented. (PV: process variables).

FIG. 3—A) The exact state of the ChemPU for each individual synthesis is represented as a graph. The nodes indicate the modules, and the edges define the tubing connections for liquid transfers and physical connections (‘has-attributes’). B) A schematic representation of the ChemPU with images of the individual modules. The ChemPU emulates the manual batch chemistry workflow and uses much of the typical laboratory hardware. The liquid handling backbone consists of an array of pumps and valves which transfer reagents, solvents and solutions of intermediates between the different units of the system. Reactions are either carried out in a round bottom flask reactor or a filter. The work-up is performed in a separator that is agitated with an overhead stirrer. The phase boundary is detected with a conductivity sensor. Solutions are concentrated in a rotary evaporator. Column chromatography is performed on a flash column chromatography machine.

FIG. 4—A representative selection of most used reaction classes in the fields of medicinal chemistry, process chemistry and total syntheses have been translated to _{X}DL and validated on a chemical synthesiser. A) Number of examples per reaction class. Once a literature procedure has been captured by _{X}DL it is marked as ‘translated’. When a translated procedure was successfully executed on a ChemPU it is moved to the ‘validated’ class of _{X}DL scripts. For reference the average frequency of reactions over the fields of medicinal chemistry, process chemistry and total syntheses is shown (lit. reference category). B) The distribution of validated reactions along a specific example for illustration is shown. C) Chemical operations, unit operations and total runtime per procedure.

FIG. 5—Representative examples of _{X}DL procedures validated on the chemical synthesiser. Transformations from all of the main reaction classes afforded products in yields comparable to those reported in the literature for manual synthesis. Additionally, multi-component reactions were performed, and a small library of compounds was prepared by varying the starting materials of one of the multicomponent reactions. Finally, the reproducibility of a validated _{X}DL procedure has been examined and a _{X}DL procedure of a multi-step synthetic sequence has been validated.

FIG. 6—The chromatography module stands out from the ChemPU hardware library in terms of the complexity of the information and material flow between the module and the controller and the other hardware. The chromatography method consists of two important parts: the sample injection protocol and the solvent gradient. Once these parameters have been optimised, the separation run can be initiated. The chemical synthesiser defines the run parameters on the commercial chromatography unit. Then the run preparations (baseline correction over the gradient, and column equilibration) are performed. Next the sample is injected onto the column. During the gradient run the chromatography machine sends the detector signals to the ChemPU controller in real-time. The controller performs the peak detection and triggers the fraction collection mechanism of the chromatography machine. When the separation run is complete the product peak is identified and transferred to the next module (usually the rotary evaporator).

FIG. 7—A schematic of the ChemPU software stack.

FIG. 8—A schematic of a chromatography system to interface with the liquid handling backbone of the ChemPU (top), and a schematic of a fraction collector extension to connect a funnel rack with the ChemPU liquid handling backbone.

### DETAILED DESCRIPTION OF THE INVENTION

The present invention makes use of two useful developments. First, the invention makes use of a standardised and precise syntax to describe the processes for a chemical synthesis (15). The description of the process steps reliably captures all the critical details for that process, avoiding ambiguity, and avoiding the need for any operator to derive any implied instructions from the instructions. Thus, any inherent teaching from an original source experimental description is provided as explicit in the instruction set.

Further, the process language that forms the instruction set is process step-focused, and the coding is developed to be independent of hardware. As such, there is a generality to the description to allow a chemical synthesiser to perform a chemical synthesis using the hardware it has available to it, rather than the chemical synthesiser

Second, the invention makes use of a universal automation platform adapted for performing the standard unit operations within the chemical arts. As such, the platform can perform many chemical syntheses, and may do so autonomously based on it reading of an instruction set, and its assembly of a virtual schedule and a virtual graph for the execution of the synthesis as the physical output from the digital input of the instruction set.

Accordingly, the invention provides a method for generating a database for chemical syntheses, where the database holds a plurality of instructions sets for a chemical synthesiser, where each instruction set is a machine code for the execution of a chemical synthesis.

In a related aspect, there is also provided the use of the database for automatic chemical synthesis, where a chemical synthesiser accesses an instruction set in the database, and performs a chemical synthesis according to the instruction set.

The invention also provides a method for generating an instruction set for use on a chemical synthesiser.

The work in the present case therefore presents the design, construction and validation of a workflow to capture the chemical synthesis literature from manual operation to a fully described and universal instruction set, or chemical description language (_{X}DL) (15, 33), suitable for execution on a chemical synthesiser. The automatic undertaking of the chemical synthesis by a suitably programmed chemical synthesiser is under the control of a Chemical Processing Unit, or ChemPU (14-16), which unit has knowledge of the physical reactionware of the chemical synthesiser and is capable of planning and scheduling the execution of the instruction set using that physical reactionware based on its abstraction of the instructions set.

The process of running the instructions set, or _{X}DL, on the ChemPU may be referred to as Chemputation, which is similar to computation, and is the reliable conversion of code and reagents into products. As shown herein, the instruction set can be compiled to run on many different ChemPU configurations, and the instruction set language can encode a wide range of synthetic procedures, which are representative of the organic chemistry ‘toolbox’.

The worked examples in the present case show the translation of over 100 different reactions, representing a variety selection of chemical reactions and reagents, into reliable instruction sets for use in chemical synthesisers. Amongst these instruction sets, over 50 have been successfully run, and therefore validated, on the chemical synthesisers with yields and purities comparable to the yield and purities reported in the originating literature from which the translations were made.

This increased synthesis throughput would not have been possible with the earlier versions of the systems described here, which could not utilise _{X}DL.(14, 16) It also signifies a massive step-up in the number of validated _{X}DL procedures compared with the seminal paper on _{X}DL (15) and is in part testimony to the increased reliability of the hardware employed in this paper.

Based on work describe herein, it is clear that chemical synthesis literature can be easily converted to a universal chemical code that can run on any robot capable of Chemputation; the only requirements for this are a batch reactor, a separator, evaporator, and purification system. This means that potentially many different robotic approaches will be able to use identical _{X}DL codes to produce identical results.

The use of a _{X}DL Chemify database will not only allow a new way to publish new procedures but provide the community with a rich source of validated data amendable to state-of-the-art machine learning for reaction optimisation, route planning, increasing safety and reducing the environmental impact of synthesis as well as dramatically reducing labour for bench chemists repeating well known procedures.

U.S. Pat. No. 5,463,564 describes the use of automated synthesisers to prepare libraries of compounds for testing. After each library is prepared, and the compounds tested and rated, the system looks to develop a structure activity relationship from the results. Using this relationship, the system then identifies the reagents that are associated with the beneficial structures. A subsequent library is then developed making use of those reagents. This includes the development of new automated synthesis instructions for the synthesis robot to follow.

Although U.S. Pat. No. 5,463,564 refers to instructions for the robotic synthesiser there is no indication that these are universal instructions that are required in the methods of the present case. Thus, the instructions mentioned in U.S. Pat. No. 5,463,564 are apparently specific for the robot that is used, and do not have general applicability for execution by other automated synthesisers.

U.S. Pat. No. 5,463,564 focuses on discovery processes, and there is no particular discussion on the use of the database as a source of synthesis information for other users to access. There is also no mention of the use of the database to provide originating, versioning, and accessing information to guide in the selection of an instruction set for execution.

Also, U.S. Pat. No. 5,463,564 does not describe how the instructions for the synthesiser are developed, and it does not seem that they are developed from a chemical literature, which is the preliminary step in the development of the database in the present case.

U.S. Pat. No. 7,164,992 describes a system for preparing polynucleotides. Essentially, the system is programmed to design the synthesis of a target polynucleotide from smaller fragments. The design procedure looks at breaking the polynucleotide down into smaller units the system knows that it can produce according to its database, which holds synthesis data and synthesis rules. There are further design rules programmed into the system to recognise what fragment terminals (e.g. overhangs) it can join to form larger sequences.

It is not clear that the database described in U.S. Pat. No. 7,164,992 holds universal instructions for automated chemical synthesisers. Context suggests that the system holds instructions that are specific to the automated DNA synthesiser. There is also no indication that the synthesis instructions are available or for sharing with other users, and there is no reference to any information that might be associated with an instruction set for the benefit of the user in selecting and validating a synthesis. The generation of the programmes for the robot from the literature is also not discussed.

### Chemical Synthesis

The present invention provides methods for converting a chemical synthesis into an instruction set for execution by a chemical synthesiser.

Typically, a chemical synthesis is a multistep synthesis. Here, the chemical synthesis may include the step of preparing an intermediate compound from one or more reagents, and the subsequent transformation of that intermediate compound into a target product. The one or more reagents may themselves be upstream intermediate compounds, prepared in their turn from suitable reagents. Accordingly, a chemical synthesis may comprise one or more chemical reactions, and preferably two or more chemical reactions.

The chemical synthesis is not particularly limited, and may encompass methods for the production of small organic compounds, metal complexes, inorganic materials including nanomaterials, supramolecular structures and polymers, amongst others.

The chemical synthesis may also encompass biological reactions, or reactions where biomolecules participate as reagents or catalysts. Such biomolecules may include polypeptides, such as proteins, including enzymes and antibodies, polynucleotides, polysaccharides, metabolites and cofactors, amongst many others.

The worked examples provided in the present case exemplify the preparation, in multi-step syntheses of small organic compounds using a chemical synthesiser under autonomous control. The worked examples also well exemplify a large series of single step reactions performed using a variety of different reagents and procedures. These basic chemical syntheses provide the solid basis for understanding that the chemical synthesiser can operate autonomously following an instruction set, and can yield products in desirable yields and purities, and at such yields and purities that are comparable to those reported in the literature for the standard bench syntheses.

The chemical synthesis may also include, and typically includes, methods of preparing a reaction mixture for a chemical reaction, as well as methods for the work-up of a reaction mixture following a chemical reaction, and methods for purifying a product of a chemical reaction.

In a preferred embodiment, the chemical synthesis comprises two or more chemical reactions, such as three or more chemical reactions. The chemical synthesis may comprise a series of chemical reactions, as well as well as parallel chemical reactions, that are later brought to convergence.

A downstream chemical reaction may be performed in a reaction vessel different to that of an upstream chemical reaction. On one embodiment, each chemical reaction may be performed in a different reaction vessel. Here, the chemical synthesis will require the transfer of material between reaction vessels as the synthesis progresses.

A chemical synthesis may comprise one or more work-up, purification or preparation steps.

For each chemical reaction in a chemical synthesis, the chemical reaction may include one or more work-up, purification or preparation steps.

### Instruction Set

The instruction set is prepared as an operating procedure for the performance of a chemical synthesis in an unambiguous manner (9-16). As such the instruction set is highly suited for use in an automated system, such as the chemical synthesisers described herein.

An instruction set may also be provided together an analytical data set, which is analytical data recorded for the product of a chemical synthesis. This data may be provided for validation of the product produced by the chemical synthesiser against the data against the data held in the database for the synthesis.

The analytical data for the product may be obtained from the literature. For example, the generation of instruction set from the natural language description of the synthesis also allow for the recording of the analytical data reported for the product of the synthesis.

Additionally or alternatively, analytical data may be recorded on a preparatory or validatory running of a chemical synthesis on a chemical synthesiser. The product that is produced on the chemical synthesiser may be analysed and the analytical data made be collected and the data may be made available to the database for collection, analysis and for onward disclosure to other operators.

A database may one instruction set for a chemical synthesis. However, and advantage of the present invention is that a database may hold multiple instructions sets for a chemical synthesis, representing variations of the operating procedure. Of particular use in the present case is the use of versioning for an instruction set. Versioning allows an instruction set to be replaced with another instruction set, whilst maintaining a record of both the original and the replacement instruction set. Versioning may be used as part of an iterative process to improve the instruction set, to allow for, amongst others, improvements in the yield and accessibility of the final product, to improve the overall process, for example to reduce process complexity or length, and also to correct errors or to otherwise reduce the amount of solvent, material, or catalyst use whilst at least obtaining the compound with the same yield and purity.

The present invention allows for an instruction set to develop beyond the instruction set immediately developed from the description of the chemical synthesis in the literature, with the possibility of providing an improved chemical synthesis beyond that originally reported.

For example, the instruction set first developed from the literature may transpose any errors or poor practice from the literature procedure into the instruction set. Any execution of the instruction set in a chemical synthesiser may then also develop and perpetuate those errors and poor practice. The development of the instructions set may then lead to the identification of those errors, and their correction, as well as the recognition of poorly practised steps, which may then be revised with appropriate steps in the instructions set.

A literature reported synthesis is also typically written for manual performance on the bench and may be optimised for or indeed limited to the techniques that are available to the bench chemist. The instruction set will operate according to the translation prepared from that description. However, using the apparatus of the chemical synthesiser the execution of the instruction set may show that the chemical synthesis does not transfer well to the autonomous system. Thus, yields may be lower than expected or the execution of the chemical synthesis may take longer than expected.

The instruction set may therefore be adapted to best run on the chemical synthesiser, optimising the optimal conditions for the chemical synthesis on the standard reactionware of the chemical synthesiser.

Thus, in the same way as computer software is versioned, instruction sets for a chemical synthesis may also be versioned.

An instruction set may represent a generalised approach for the chemical synthesis of a class of target products.

For example, for a reductive amination, the instruction set may provide detailed and unambiguous process instructions, but these may be applicable for a wide range of different amines and different aldehydes, optionally together with a range of different reducing agents. Here, whilst the instructions set provides clear, unambiguous and detailed procedural information for execution by the chemical synthesiser, such may be applied to any of the combinations of reagents.

An instruction set may be provided within a database for persistent storage and it is available for access and downloading to a chemical synthesiser on demand.

The generation of an instruction set may have an origin in a literature reported chemical synthesis, which is described in natural language. Such chemical syntheses may be provided in journal articles, for example within the experimental section or the supplementary information (SI), or may be provided as an experimental standard, for example within standard texts or protocols. This information may be proved on hardcopy, or in electronic form, for example with a PDF or provided on web page.

This natural language is converted to a machine-readable format which is the instructions set for use in the present, and such a translation may follow the process previously described by the present inventor in WO 2021/219999, the contents of which are hereby incorporated by reference in their entirety.

Alternatively, the generation of a new instruction set may be based on an earlier prepared instruction set. Here, the new instruction set is a variation of the earlier set and

The new instruction set may be intended to optimise or otherwise improve the results from the execution of the earlier instruction set.

### Chemical Synthesiser

The chemical synthesiser is the chemical apparatus that performs a chemical synthesis following an instruction set. The chemical synthesiser may do so autonomously, generally only requiring the loading of appropriate reagents, solvents and catalysts by an operator. It is the operator how may select the instruction set for the chemical synthesiser to work to, but this is not essential, as the chemical synthesiser may autonomously select instructions sets, or may be them selected for is, for example where a suitably programmed database providing suitable instructions sets.

A chemical synthesiser may have available to it a series of modules for undertaking common tasks within chemical syntheses.

Chemical reactions typically share many of the same process features, and a laboratory chemist will learn a series of standard techniques, often making use of a series of standard apparatus.

It follows that many and most chemical reactions may call for only a small selection of hardware for their performance, and for the subsequent steps of work-up and purification.

A chemical synthesiser may be provided with the hardware to undertake the standard series of techniques, and these may correspond to the standard apparatus that is used in the laboratory, or such apparatus as might be adapted for use in an automated chemical synthesiser.

At its most general, a chemical synthesiser is provided with reactionware that includes at least one or more of, and preferably all of, a reactor, a separator, an evaporator and a purification system. Each of these may be referred to a module in the chemical synthesiser.

The reactor incudes a reaction vessel for performing reactions and for storing of materials, for example prior to their transfer to another module within the synthesiser.

A reaction vessel may be provided with means for agitating a reaction mixture contained within. Typically, the contents of the reactor may be mechanically agitated, and an overhead stirrer or a shaker may be provided for this purpose, amongst other possible options

The separator is for separation of immiscible fluids, such as the separation of organic and aqueous phases. The separator may be provided with a phase analyser to locate or otherwise identify a phase boundary between liquid layers. The data from the phase analyser may be used by the chemical synthesiser to control flow through the separator to allow for the separation of phases passing through the separator.

The evaporator is for the removal of components from a mixture by evaporation, such as the removal of solvent, although products, reagents and/or by-products may also be removed by evaporation. The evaporator may include a heater or cooler for control of temperature, and may also be provided with vacuum. The evaporator may include means for agitating the mixture, for example to distribute the mixture in the flask or to increase the surface area.

The chemical synthesiser may be provided with a transfer system for the movement of components between units. The transfer system may be fluid-based, such as liquid or gas, and may also be solid-based.

Thus, in one embodiment, a chemical synthesiser may comprise one or more units for reaction and mixing, separation, filtration, heating, cooling, pressurisation, chromatography, analysis and material transfer.

An operator may be required to load a chemical synthesiser with the reagents, solvents and catalysts as might be needed for a chemical synthesis. The chemical synthesiser, working to the instruction set, may instruct the operator on the identity and amounts of material that are to be provided.

An operator of a chemical synthesiser may have minimal interaction with a chemical synthesiser during the execution of an instruction set, and may only be required to interact with the chemical synthesiser when the synthesiser self-reports and error in the execution of the instructions set, for example in view of anomalous analytical data, or in view of failure, such as electrical or mechanical failure, of apparatus within the reactionware.

The chemical synthesiser is suitably programmed to execute an instruction set using the reactionware that is available. Once the chemical synthesiser receives an instruction set, the ChemPU, the processing unit of the synthesiser responsible for control and execution, draws up a schedule for the synthetics steps within the synthesis. The schedule takes the operations from the digital instruction set input and matches the operations to the available reactionware, as represented as a graph in an abstract layer of the processing unit. The ChemPU has an understanding of the status of all reactionware with the synthesiser—which status is known from analytical readings or from the know use of those reactionware from subsequent uses, and the execution schedule is drawn up accordingly.

Through the chemical synthesis following the instruction set operations in the local reactionware, the ChemPU monitors the state of reactionware against the schedule and plans instructs operations within the synthesiser at appropriate times and with appropriate movements of material between reactionware modules.

### Chromatographic Separation

The chemical synthesiser preferably includes a chromatography system, which is provided as part of a purification system.

The worked examples in the present case demonstrate the use of chromatography, and specifically column chromatography, as a system for autonomous separation of a target product from a reaction mixture. The system can automatically determine the Rf and the correct solvent to use.

Thus, where a literature procedure requires separation of components, such can be implemented on the chemical synthesiser using chromatography.

Typically, a literature procedure for chromatographic separation will stipulate the stationary phase, as well as the eluting phase, and any change in the eluting phase, such as reverse or normal.

The chromatography may be column chromatography, for example with silica gel or alumina alumni as the solid phase.

It is preferred that the chromatography system uses pre-prepared cartridges for use in the system, and the system may have a plurality of such cartridges, where these cartridges may differ in their size or phase. Such cartridges are readily available from commercial sources, or may be prepared by the operator, although this is less preferred and not needed where the commercial supply is readily available.

In the worked examples of the present case, a Buchi Pure C-815 chromatography system is incorporated into the chemical synthesiser for use as a chromatography system.

The chromatographic system typically includes a loading system for placing a sample on the column, as well as an analytical system for detecting components eluting from the column, or for analysing fractions collected from the column.

The chemical synthesisers for use in the invention are typically provided with a fluidic system for the transfer of fluids between modules. Thus, the chemical synthesiser is provided with fluid lines, manifolds and valves for the controlled distribution of fluids. Such may be used to transfer mixtures from another module, for example from a rotary evaporator module to a column in the chromatography system. Subsequently the fluid system may then be used to provide the eluent to the column to allow passage of the components within the mixture through the column.

In the worked examples of the present case a UV-vis detector is used to analyse the components eluting from the column.

In the usual way of chromatography systems, the eluant may be evenly distributed across collection vessels, where each of the collected fractions is analyse. Alternatively, the detector is used in real time to analyse the eluant, and the eluant is distributed across different collections vessels in response to changes in the analytical data.

The system may also allow for other standard practical steps that are associated with the performance of column chromatography, such as the rinsing of a reactor following mixture transfer to the column, and the transfer of the rinse onto the column to minimise loss of material. Such procedural steps are in-built into the procedure executed by the chemical synthesiser from the coordination of the digital instruction set with a scheduler in the chemical synthesiser.

In addition, or as an alternative, to UV-vis detection, an elastic light scattering detector (ELSD) may also be provided and used for the detection of components eluting from the column, for example where UV-vis detection is not appropriate or helpful.

The incorporation of chromatography into the chemical synthesiser expands the range of chemical syntheses in the literature that are accessible to the synthesiser. Accordingly, the chemical synthesiser of the invention has a far greater practical application.

### Database

The present invention provides a database for storage of an instruction set. The instructions set is available from the database on request, and may be downloaded to a local chemical synthesiser as required for a chemical synthesis.

The database may be hosted by any suitable computer, and this computer may be remote from the chemical synthesiser. The database may be held in the cloud.

The database is a persistent store for instruction sets, and permits ready and easy access to operators of chemical synthesisers.

The database may have certain access restrictions, such that the instructions sets, either in their entirety, or only a sub-group of the instruction sets, is available to a licensed operator, such as a subscriber to the database.

A database may include, and preferably includes, a plurality of instruction sets. A typical database may hold 100 or more instruction sets, such as 200 or more instructions sets, such as 1,000 or more instructions sets.

The database may be searchable. The instructions set within the database may be organised or otherwise categorised to aid searching and recall of data, and for the purpose of data analysis for the database rights holder.

A database may include a categorisation of instructions sets by target product, including both specific and general target products.

A database may include a categorisation of instructions sets by reagents and catalysts, including both specific and general reagents and catalysts.

A database may include a categorisation of instructions sets by classification of the formal bond-formation in the synthesis step or steps.

A database may include a categorisation of instruction sets by reaction classification.

A database may include or request the location data of a chemical synthesiser, as well as the precise timings for the undertaking of a chemical synthesis performed by a chemical synthesiser. Location data information may be used as a selection criterion for the presentation of available instruction sets to an operator. Certain target products, or the use of certain reagents and intermediates, may be restricted or illegal in certain jurisdictions, and the database may restrict access to those instruction sets associated with these target products, reagents and intermediates.

Instruction sets held on the database may differ in one or more of their target product, their reagents and catalysts, their formal bond formation, their reaction classification, or the number of reactions in the synthesis, amongst others.

The presentation of available instructions sets to an operator may include the pre-selection of instruction sets based on the available reagents, solvents and catalysts available to the operator of the chemical synthesiser. Available in this context, may include the availability of such materials that are present as reagents, solvents and catalysts within the chemical synthesiser itself, or the availability of these materials within the laboratory of the operator, or more generally the availability of such materials on the open chemical market (with further selections available to accommodate the readiness of any supply).

It is an advantage of the present invention that the instruction sets are generated independently of physical hardware, as they describe process steps that are not prescriptive of the reactionware that is to be used for the chemical synthesis. As such, it is not necessary for an operator to stipulate what reactionware is present within the chemical synthesiser as the chemical synthesiser may be capable of performing standard chemical operations using its collection or reactionware modules, such as the reactor, the separator, the evaporator and the purification system.

However, it is anticipated that certain instruction sets provided within the database may require bespoke, unusual or advanced chemical process steps, and such might require reactionware that is not part of the standard suite of reactionware for a chemical synthesiser.

Such instruction sets may be presented only where an operator has available a chemical synthesiser capable of performing process steps having the suitable reactionware to perform such steps.

The chemical synthesisers for use in the present may be provided with a chromatographic system for the separation of components in a mixture. Such may be used to isolate intermediates for use in downstream reactions within the chemical synthesis, or may be used to isolate a target product in a desired yield. Such processes are commonly use in the literature, and may be translated into the instruction set.

The database may be used to present only those instruction sets that are available for execution by the chemical synthesiser. The chemical synthesiser itself may provide the database with its reactionware index, for the database to present instructions sets that are performable on

The database may also present to the operator those instruction sets that could be performed on the chemical synthesiser if the chemical synthesiser were to be supplemented with a particular reactionware module.

Accordingly, the operator is shown the instruction sets that are potentially available should the necessary adaptations to the chemical synthesiser be made.

An advantage of the chemical synthesiser is its modular construction, and its use of fluidic control, including liquid and gas control, and solid control to allow transfer of materials through the modules that are present. Additional reactionware modules, which may be provided for particular unit operations, are readily accommodated into the system.

The database may be expandable, and new instructions sets may be added to the collection either by the database rights holder or by authorised users of the database.

The database may additionally hold information of each instruction set for the benefit of operators and for the benefit of the rights holder. The database may also permit the gathering of user data in relation to the viewing and downloading of instructions sets, and optionally, the database may acquire feedback from a chemical synthesiser executing an instruction set, including analytical data, or from the operator directly.

For example, the database may record the number of times an instruction set is viewed or downloaded. In this way, popular instruction sets become visible to other operators.

The database may also record the originator of the instruction set, together with the identity of those operators who have validated or revised the instruction set.

In this way, tested or familiar originators and validators can be recognised, and an operator may select an instruction set based on the identity of the originator or the validator. Originators and validators may be ranked based on experience, or the quality or relatability of their instructions sets, or in connection with the number of instruction sets downloaded by operator for which they are the associated operator or validator.

The database may also record the user ranking of an instruction set. Thus, once an instructions et is executed and the result known, an operator may provide ranking of the instruction set based on perceived ease of execution and the quality of the results. Such rankings can be made available for instruction sets, and other operators may be free to select on instruction set over another based on the user rankings of those instruction sets.

The database may also provide information on the operators themselves, for example to show which operators are regular or trusted users of the database and the instruction sets. Such operators may be regarded as valued users with access to data and information not available to infrequent users. Any operator identifying information may be obscured for data protection.

The database rights holder can assemble such information and optionally make information available to the chemical synthesiser operators. The use of user and creator data in this way matches the use of user and create data on media platforms, for example, including social media platforms. Thus, the database can provide community opinion on instruction sets, and also the community is able to suggest edits and improvements to those instructions sets (wiki-style modification of the instructions sets).

As noted previously, an instruction set within a database may be provided together characterising data for the chemical synthesis. This original of this data may also be presented.

The characterising data may include information reported in the literature for the chemical synthesis. This can include analytical data, as information on the scale and yield of the synthesis.

The characterising data may include information derived from a repeat of the chemical synthesis by a chemical synthesiser following an instruction set. This is validated data as it represents the data that will likely be obtained for an operator repeating that synthesis using the instruction set in the operator's own chemical synthesiser.

For each chemical synthesis there may exist a plurality of different instruction sets. These instructions may represent the different conditions reported in the literature for a particular synthesis. These may differ, for example, in scale, choice of solvent, catalyst and reaction conditions (temperature, atmosphere and so on). The use of the database, with operators selecting certain instructions sets, may show that one instruction set if favoured others, and this information is useful to other operators.

Multiple instruction sets may also be provided as part of series of improvements of an original instruction set. Such a series may show the optimisation of the instructions sets, and the database may record what advantages are available through the alteration of the instruction sets.

The database is provided for operators to upload their instructions set as an alternative, such as an improvement, to an instruction set held on the database for a particular synthesis posted.

The database may distinguish between those instruction sets that have been validated, and those that have not. The validation may be a verification of the instruction set by the database rights holder, or it may be an approval of an instruction set by other users of the database, such as other operators who may be provided with approval rights based on their experience and history (for example, in submitting additional instructions sets that have been consistently approved).

Instruction sets that have been validated by the rights holder or community members may be regarded as more trustworthy than those instruction sets that carry no validation.

The database might hold details of all the failed reactions and negative data including the analytical data presenting the outcome of the reaction.

The database may provide a list of instruction sets that require validation, and these may be offered to users of the database for validation. The database may offer a reward system where users who repeat unvalidated instruction sets are given credit for their work, or may be given rights as a validator for their work.

The database may also hold instruction sets that are known failures or have a high probability failure rate, and are recoded and validated as such. Such information is usefully provided to show to an operator what operations and conditions within a chemical synthesis are problematic, so that they may be avoided or may be investigated for the purposes of optimisation and improvement.

The information on the failure may also report the nature of the failure its consequence for the synthesis, which might include abandonment of the synthesis or obtaining the target product at a yield or purity that are deemed unsatisfactory.

The information on failures may also report where those failures are not specifically related to the operation steps, that is not related to chemical process embodied in the instruction set, but related to the operation of the reactionware itself within the chemical synthesiser. Such information may reveal where the reactionware is prone to breakages, or is suspectable to variation in its operation, or may reveal that certain operations are operationally too complex or time-consuming for the chemical synthesiser to complete. This information may be usefully collected and may inform the use or reactionware within a chemical synthesiser or may inform the operator on the types of chemistry that can be reasonably undertaken on the chemical synthesiser.

As described above, a chemical synthesiser contains a basic set of reactionware that permit may standard experimental procedures to be undertaken autonomously by the synthesiser.

Thus, the collection of information on failures is as useful as the information on optimisation and success.

The database rights holder may charge for access to an instruction set. The database rights holder may provide access to part or all of the database for a regular charge. The database rights holder may provide access to only part of the database for a basic fee, with other parts of the database accessible for an additional or premium fee. The database rights holder may choose what parts of the database, and therefore what instruction sets, are made available under difference payment and licence models.

The selection of an instruction set from the database may be the free choice of the operation of the chemical synthesiser, and that selection is made based on the product that is desired and with guidance on the types of chemical syntheses that are possible with the chemical synthesiser available, and the types of chemical material, such a reagents, solvents and catalyst, that are available

In other embodiments, the chemical synthesiser may select an instruction set for use. The chemical synthesiser may be suitably programmed to do so. The chemical synthesiser may also operate under the instructions of the database, which may select an instruction set for the synthesiser to use. Here, the operation of multiple chemical synthesisers may be coordinated as part of a community synthesis project.

The data included can be run-time telemetry data and also end-point analytical data as well as purification method validation.

### Other Embodiments

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### Experimental Section

We designed and built a _{X}DL database called Chemify for 100 entries and anticipate this will rapidly expand and is available for anyone to run and validate on suitable hardware. Not only can these _{X}DL entries be validated over a wide range of different systems, but statistics can also be gathered, and new versions suggested, if required.

In addition to directly repeating the validated procedures, the substrate scope for each _{X}DL can be gradually expanded by changing the substrates and adjusting key parameters—such as temperature or time—of the reaction while keeping the rest of the process unchanged. Since we have selected reactions based on popularity, the resulting set of validated _{X}DLs covers a substantial range of common reactions and constitutes an entry point to the automation the entire organic synthesis ‘toolbox’.

Further—through the acid test of performing >50 procedures of highly diverse chemistry—the hardware and software of the ChemPU has been pushed to the limit and a path to full universality demonstrated. To do this, key advances have been made by incorporating a _{X}DL-enabled flash column chromatography system in the hardware library. This means that the chemical synthesiser can not only perform the reaction, work-up and concentration, but also the chromatographic separation of the product and directly deliver the purified compound on demand. To achieve this, we show that the platform can react in a dynamic manner responding to detection of the product and collecting the appropriate product fractions demonstrating automatic synthesis with autonomous purification—all in a single system.

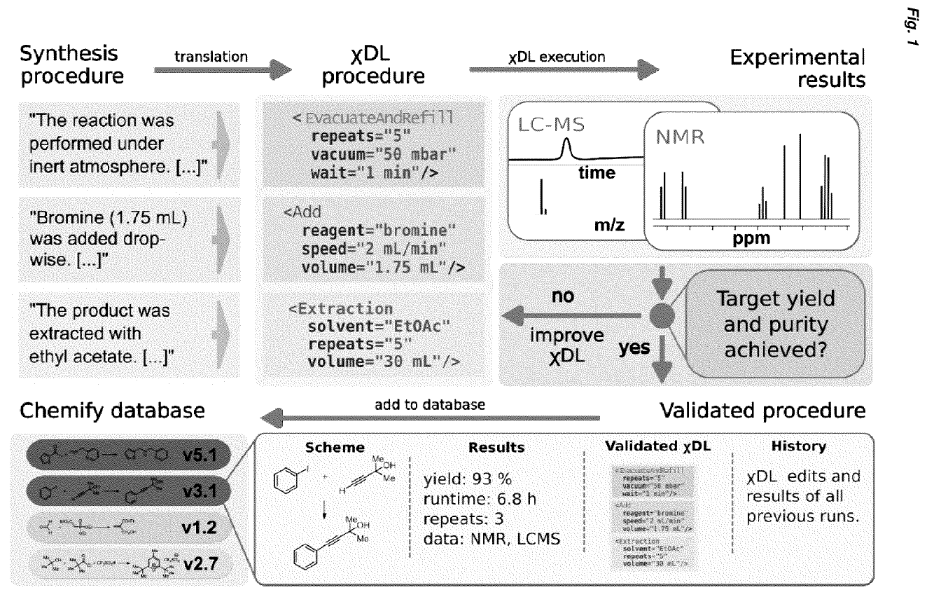

The workflow starting from a literature procedure to a validated entry in the Chemify database is illustrated in FIG. 1. As opposed to earlier work on _{X}DL, the focus was not on an exact translation of the original procedure text to _{X}DL but the implementation of a chemical process giving the target molecule. Following this approach allowed use not only to reproduce the literature but to improve the processes in several instances. Chemical reactions can be captured in _{X}DL, which represents synthetic steps as sequences of physical processes such as Add, Dissolve, Evaporate etc. There are currently 44 steps within the _{X}DL framework, each step having a fully customizable set of parameters. All often-used tasks in organic syntheses have a boilerplate _{X}DL step to represent them such as EvacuateAndRefill to establish an inert atmosphere or Separate to perform a liquid-liquid separation and extraction. The _{X}DL steps help to enforce precise descriptions of the process and eliminate any ambiguity such as the number of cycles of evacuation and inert gas refill or process-critical addition speeds. To achieve this, we used our web-based Chemistry Development Environment (ChemIDE)(33)—that aids the quick generation of _{X}DL procedures by providing a text-to-_{X}DL translation tool. This works using a template library of all available _{X}DL steps, and an editor where individual _{X}DL steps are represented as graphical elements, which can be edited and arranged as needed. (33) ChemIDE was used in the generation of all _{X}DL procedures detailed in this work.

Expression of a chemical procedure in _{X}DL does not immediately solve the problem of missing information or ambiguity present in the original prose instructions, but it does provide an unambiguous path to close it. To do this some process development and iterations may still be required to maximise yields and purity. After appropriate analysis (NMR/LCMS/GCMS) of the target compound from the chemical synthesiser execution of the _{X}DL code, an assessment on the quality and purity of the product is made. If necessary, the _{X}DL is improved to increase the yield and purity and executed again. The key advantage of _{X}DL is that once a successful process has been encoded, all subsequent users who execute the code on compatible hardware can expect identical results, with no more requirement for process development. All the critical knowledge to execute the process on qualified hardware, both tangible and intangible, is now captured in the _{X}DL. At this stage the protocol can be added to the database as a validated process, backed up by the full characterisation of the target product and the process development history. Including the process development history is a distinguishing feature of the Chemify database. By showing the results of less successful experiments and contrasting them with the final successful run critical aspects of the process are highlighted and can be quantified.

The Chemify database persistently stores and maintains information for _{X}DL procedures, experimental results, and relevant analysis. It is a locally hosted PostgreSQL database server containing all validated _{X}DL scripts as described above, that can be accessed via ChemIDE—the web-based _{X}DL development environment or using a Python 3 based API for automated database querying. Moreover, for end-user experience, ChemIDE is equipped to display characterization parameters of each experiment such as product scale, yield, status (translated, validated, failed) and process duration). Users can submit, search, download, and reproduce trusted syntheses.

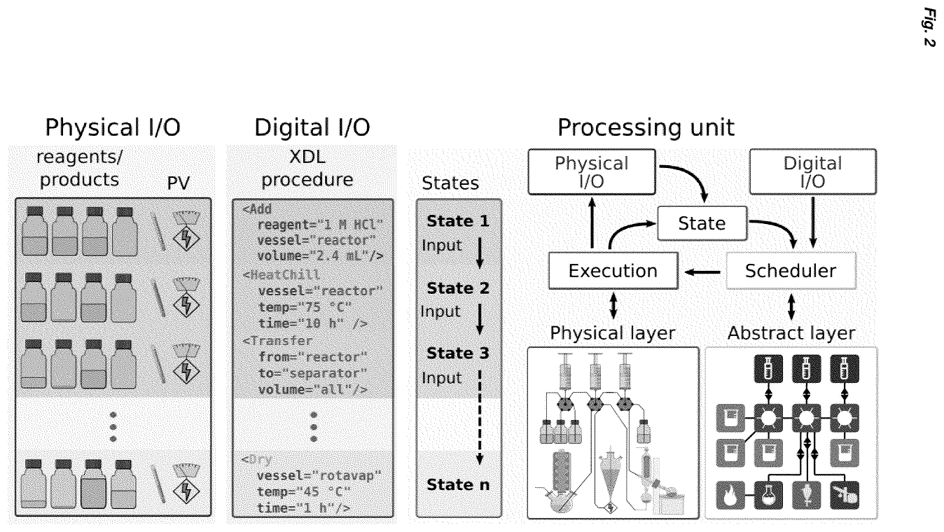

The database contains successful, final validated synthesis scripts as well as previous developmental versions, which may work to a varying degree—affording the desired products in lower yields, insufficient purities, or leading to process failures (for example, causing blockages or formation of emulsions during liquid-liquid separations) due to insufficient or incorrect description of the necessary process parameters for automation. Comparing failed, or lower yielding experiments, to successful attempts of a given specific reaction or reaction class can unveil critical aspects of the process. Further, the database also contains _{X}DL entries that have been translated, but not yet executed on a suitable automation platform. Users interested in unvalidated _{X}DL files can access these and have the option to validate them. The _{X}DL procedures reported here have been validated on a chemical synthesiser, a chemistry automation platform that emulates the manual operations of the bench chemist as far as possible. While operationally simple and intuitive, the rigorous implementation means that the platform operates as a finite state machine, see FIG. 2. It can be in one of a finite number of states and it transitions from one state to the next based on well-defined operations. These operations are defined by the program—the _{X}DL synthesis protocol—and the sensor feedback—e.g., temperature, conductivity, pressure, or UV absorbance.

The direct mapping of the _{X}DL synthesis instructions to state transitions, or‘unit operations’, highlights the rigorous abstraction of synthesis processes in _{X}DL. Moreover, the clear definition of state transitions defined in the _{X}DL procedure is critical to ensure the reproducibility of the _{X}DL synthesis—including on different layouts of the Chemputer and potentially entirely different, qualified hardware set-ups.

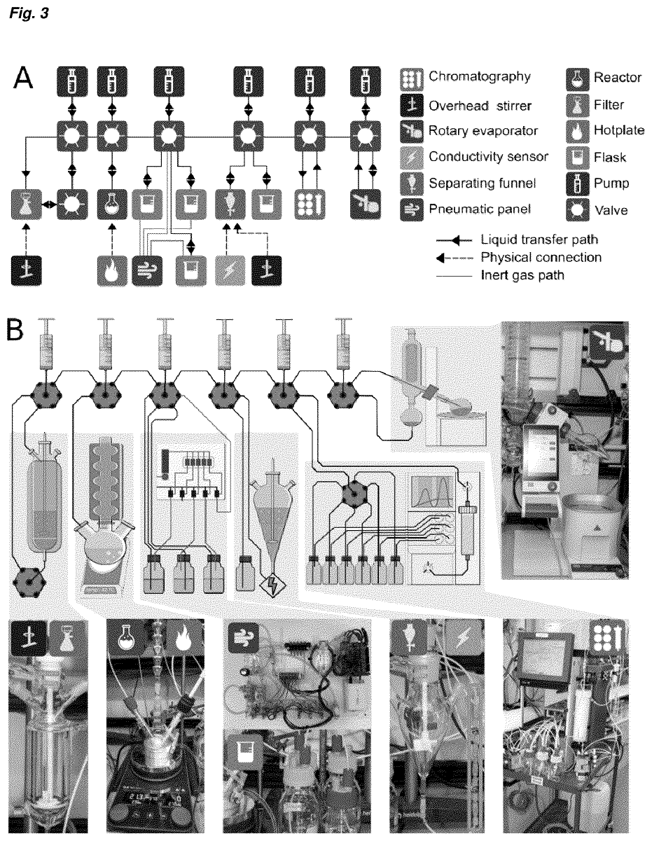

The ChemPU state machine consists of three logical parts: the physical input/output (I/O), digital 1/O and the processing unit. The processing unit can exist in several states with each state being a result of either the initial conditions of the ChemPU or a combination of the physical and digital I/O, that is, the current conditions as defined by the sensors, the process variables and the _{X}DL step being executed. The execution of the _{X}DL step according to the scheduler gives rise to a new state to be acted upon in later steps and results in a physical change to the physical I/O e.g., a change of location of reagents, a change in temperature, the phase boundary in a liquid-liquid separation or the peak elution during chromatography. The scheduler resorts to a graph representation of the hardware (abstract layer) to interpret the _{X}DL script and orchestrate the hardware for concerted tasks (e.g., moving liquid through the liquid handling backbone). The abstract layer defines the locations and connections of the hardware devices as nodes and contains specific information on each node such as the IP address and temperature limits of the device in question. The graph file together with the _{X}DL file can be compiled into an execution file (executable _{X}DL or xdlexe), which is platform specific. The strict separation of the description of the chemical process into the _{X}DL file and the hardware platform description into the graph file ensures that the _{X}DL file remains platform independent. It also allows for flexibility in how the platform is designed and what its exact physical layout is. This means that each _{X}DL can be versioned and compiled to run on any suitable platform and the ChemPU system is highly modular, flexible, and extensible, see FIG. 3.

By mirroring the unit operations of batch synthetic chemistry, the chemical synthesiser represents a universal, programmable hardware platform for execution of synthetic chemistry. The platform can be readily expanded due to its modular nature, with individual modules being connected via the liquid handling backbone, analogous to the bus of a conventional computer. Connection to the liquid-handling backbone (including pumps and valves) is via a single piece of flexible tubing, allowing modules to be easily removed for maintenance or rearranged to optimise operations e.g., by segregating aqueous and water sensitive parts of the process. The liquid handling backbone consists of a series of syringe pumps and valves. A typical backbone consists of six of each, however the backbone is readily contracted or expanded to accommodate the requirements of any chemical process. The valves are six position, seven port valves. Each valve is connected to its nearest neighbour(s) and a waste container and can connect to three to four different reagents, solvents, or hardware modules. The connectivity of the modules to the backbone is represented in an abstract manner by a graph as described above. Cleaning of the backbone is carried out via an automated cleaning routine that can be defined by the user to account for different types of contamination present after different procedures. In addition to the liquid handling backbone the chemical synthesiser systems used to execute the syntheses reported here used a reaction module consisting of a standard hotplate controlled via an Ethernet-to-serial convertor, a separator for liquid-liquid extractions equipped with an overhead stirrer for agitation, and a conductivity sensor for phase boundary detection, a jacketed filter for precipitation and recrystallisation of products, a number of reagent flasks, a rotary evaporator and an optional chromatography system.

With the abstraction of Chemputation, the _{X}DL language, and the chemical synthesiser platform we set out to translate and automate the typical reactions from the organic chemistry ‘toolbox’. Organic chemistry encompasses an immense diversity of transformations. Despite the variety, most reactions can be classified succinctly with less than ten categories. Several studies have analysed the reaction frequencies in different fields e.g. medicinal chemistry, process chemistry, and total synthesis. (34-37) There are some notable differences in the distribution of reaction classes utilized in synthesis, depending on the primary goal e.g., medicinal chemistry research may prefer transition-metal catalysed C—C bond forming reactions, which allow for a convenient generation of large numbers of related compounds for biological assays, while modern total synthesis is likely to contain more elaborate ring-forming reactions for assembly of complex molecular scaffolds.(38)

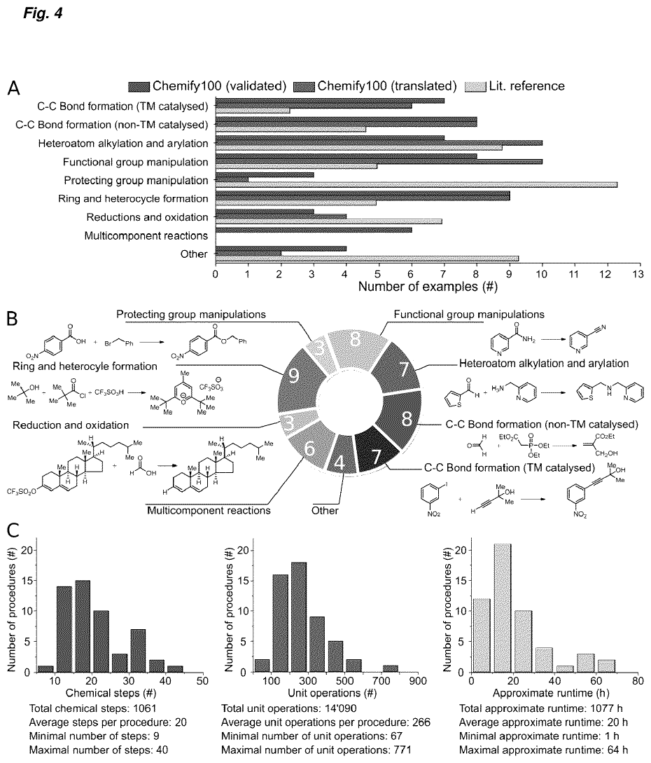

Additionally, while protecting group chemistry is the cornerstone of some synthesis fields, such as peptide synthesis (39) or carbohydrate chemistry,(40) researchers working on total syntheses often prefer more elegant protecting-group-free approaches.(41) Despite the minor variations, these categories embody the varied toolbox of modern organic chemistry. To represent all these classes with the examples from all these types of reactions we chose to translate the _{X}DLs of these procedures and validate them with the chemical synthesiser, see FIG. 4. For clarity, the carbon-carbon bond forming reaction class was further separated into transition-metal catalysed and transition-metal-free reactions. Furthermore, a separate multi-component reaction class was introduced as these reactions generally accomplish multiple chemical transformations in one synthetic operation. The initial reaction were chosen from the most cited papers from the journal Organic Syntheses.(42) This journal is unique in the organic chemistry field in that it publishes practical methods for synthesis of either notable compounds or execution of important synthetic methods, and the submitted procedures have been repeated at least once by expert chemists other than those who submitted the original synthesis. Although the procedures from this journal generally have a high level of detail, there was still the need for some process development, highlighting the difficulty to capture all necessary information in an unstructured prose text format as opposed to _{X}DL. Selecting these most cited papers from Organic Syntheses covered the top reaction classes but gave an uneven distribution. Hence, further examples were manually selected from notable literature sources to achieve a more balanced representation of the organic chemistry toolbox with our dataset.

The reactions chosen for each of the categories include well-established classical reactions, important contemporary reactions, as well as some more unconventional synthetic transformations (FIG. 4). The selected transition-metal catalysed carbon-carbon bond forming reactions include commonly used Suzuki, Heck, and Sonogashira couplings, as well as a stereoselective Carroll rearrangement.

The transition-metal-free carbon-carbon bond forming reaction class encompasses such classical reactions as the Wittig reaction, Friedel-Crafts alkylation, Aldol and Claisen condensations. Different types of heteroatom alkylations are represented by palladium-catalysed Buchwald-Hartwig coupling, copper-catalysed alkylation, SNAr reaction of heteroarenes, and reductive amination reactions. Functional group interconversions contain examples of a Mitsunobu reaction, nitrile formation, esterifications and others. Manipulations of protecting groups include the common protecting groups, such as the Boc, benzyl, and tosyl groups. Ring and heterocycle formations, include both classical syntheses such as the Fischer indole synthesis, as well as a more exotic formation of a trisubstituted pyrylium salt. Reduction and oxidation reactions contain conventional hydride reduction, Jones oxidation, and a palladium-catalysed hydrogen transfer reaction. Finally, the multi-component reactions contain the well-known Ugi reaction besides other more unusual cascade reactions and one-pot multi-step manifolds. This diverse set of reactions covers the standard organic chemistry toolbox. Crucially, automating further reactions just requires translation of the original synthesis procedure to _{X}DL.

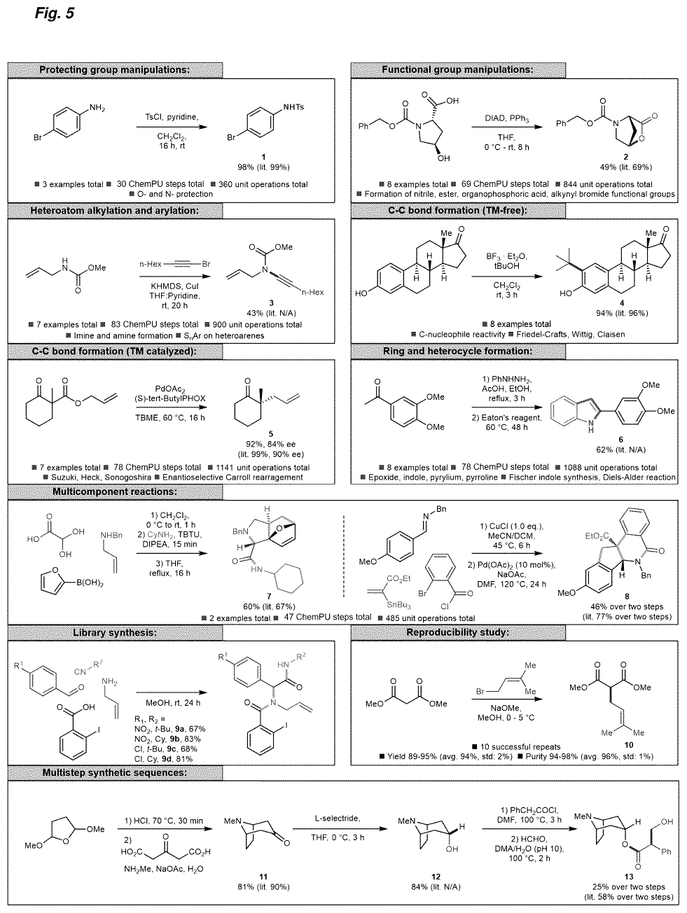

Overall, we were able to do more than 1061 chemical steps (i.e., one _{X}DL instruction block) (FIG. 4C). This and the following figures include only the operations from the final iteration of each _{X}DL protocol. All steps and operations that were executed in less successful runs are not counted for the purpose of this discussion. The average procedure consists of 20 such discrete, high-level instructions with some procedures reaching 40. Unpacking these high-level _{X}DL steps into the corresponding unit operation—e. g. StartStir, WaitForTemp, ApplyVacuum—gives a total of 14,090 operations that have been executed. The successful executions of all _{X}DL scripts took more than 1000 hours of Chemputation across 7 different systems. This figure includes the reaction time but does not account for asynchronous steps, i.e., steps where two processes are running in parallel on the same chemical synthesiser hardware, such as for example a cleaning step for a rotary evaporator during the reaction time. The yields of the reactions performed on the chemical synthesiser were in general comparable to the literature yields after a short period of process development. This could be required to fill the gaps in the original protocol and is common to all synthetic development, manual or automated or to adapt elements of the protocol that were not amenable to automation e.g., unexpected formation of precipitates that lead to blocked lines. A selection of reactions is shown in FIG. 5 to illustrate the performance of the platforms and give specific examples to show the breadth of chemistry that has been performed.

The independence from the exact type of chemistry being executed allows the chemical synthesiser to be a general synthesis platform, and reactions utilising a diverse set of reagents and conditions were automated on the same hardware. It is noteworthy that the system is tolerant of moisture-sensitive or highly reactive reagents, such as potassium bis(trimethylsilyl)amide (KHMDS) used in a copper-mediated alkynylation of a carbamate to afford 3, boron trifluoride used in a Friedel-Crafts alkylation of a steroid estrone to afford derivative 4, or Eaton's reagent (10% phosphorus pentoxide solution in methanesulfonic acid) used in a Fischer indole synthesis of 6. Additionally, reactions requiring inert atmosphere were successfully executed on the platform, including a palladium-catalysed enantioselective Carroll rearrangement to give 5. Procedures of up to 90 mmol scale were efficiently executed on our ChemPU platform. Conveniently, once a _{X}DL script is produced, a particular reaction can easily be scaled up or down within the constraints of the available vessel sizes and the chemical process such as safety considerations, heat- and mass-transfer. The _{X}DL procedures for the generation of more complex products arising from multicomponent and cascade reactions were also successfully executed on the platform. For example, a Petasis/Diels-Alder cyclization cascade has been used for rapid generation of a scaffold containing multiple stereogenic centres 7, with potential for further derivatisation in a library synthesis. Similarly, a copper(I)-catalyzed three-component coupling/palladium(0)-catalyzed annulation cascade was also successfully utilised affording product 8 containing the indenoisoquinoline scaffold. One particularly attractive prospect is the use of validated _{X}DL procedures for the construction of large libraries of compounds for biological screening. A representative set of examples of _{X}DL procedures validated on the chemical synthesisers is shown in FIG. 5.

Such libraries could conveniently be accessed by only changing the starting materials and without major modifications to the synthesis scripts i.e. once a process has been established it can be applied to many different substrates as a general procedure by only varying key parameters, such as the substrates, reaction solvent and reaction time. To showcase such an approach, a small library of α-acylamino amides 9a-9d was synthesised via a multicomponent Ugi reaction. To do this we conducted simultaneous execution of multiple or ‘multi-threaded’ reactions in parallel on the ChemPU by using reactant combinations from two different isocyanide and two aldehyde starting materials affording four structurally related a-acylamino amide products. Further expansion of the set of reactants used would rapidly expand the number of the products generated, allowing for rapid generation of larger libraries.

To examine the consistency and reliability of executing the curated _{X}DL procedures, we set out to repeat the same reaction protocol multiple times on the chemical synthesiser platform. An alkylation of malonate ester, affording 10, was chosen as a suitable reaction for the reproducibility study as accurate temperature control and rate of addition are key to the success of the process. After the initial process development, a validated _{X}DL procedure script was obtained, and the reaction protocol was successfully replicated 10 times in 12 attempted runs. The two failures were caused by incorrect phase boundary determination during liquid-liquid separations: product could have been recovered through manually restarting the system, but that was not done here. Crucially, execution of the curated _{X}DL procedure reliably afforded the product in consistent yields (avg. 94%, min 89%, std: 2%) and purities (avg. 96%, min 94%, std: 1%). Together with the ability to generate libraries of compounds, the system can be used to automate the highly repetitive work of generating multiple batches of the same material or repeating the same reaction with different substrates once the initial protocol has been set-up.

The versatility of the platform is further demonstrated by the ability to execute multi-step synthetic sequences. Atropine 13, an anticholinergic medication used in treatment of nerve agent poisonings, was synthesized in four steps from simple commercially available starting materials. Notably, synthetic protocols for individual steps from multiple sources, as well as a reduction protocol that was previously reported only for related substrates but not for the synthesis of 12, were successfully converted to _{X}DL procedures. The ability to efficiently execute multi-step reaction protocols, combined with the reliability offered by reproducible execution of a well-defined synthesis script, reaffirms the universality of the platform towards the breadth of synthetic organic chemistry.

Chromatographic separation of the product compound from a reaction is the go-to method of purification for small and medium scale organic syntheses. Many commercially available chromatography systems exist that assist the lab-based chemist in chromatographic separations. However, these systems still require a significant amount of user interaction. For example, the crude material must be manually loaded onto the column and the product fractions must be manually identified, washed out of the fraction vials, and combined. Further, the commercial systems require user interactions at several different stages, thus tying the chemist to the lab even if it is only for a trivial task such as loading the sample onto the column. To integrate the Buchi Pure C-815 chromatography system with the ChemPU, two auxiliary hardware units were built: A column carousel that allows pre-installing different columns on the system and an extension to the fraction tray. The extension to the fraction tray allows for recovery of the product fraction by the chemical synthesiser. The first challenging-to-automate operation is the sample loading onto the column. The laboratory-based chemist usually chooses between dry-loading and liquid injection of the sample. We aimed to implement the liquid injection method which ties in nicely with the liquid handling backbone of the chemical synthesiser. The liquid injection sample loading method requires little process development, requiring only the identification of a suitable solvent mixture and volume to dissolve the crude material. The second challenge to full automation of normal-phase chromatography is to reliably select the product peak. Usually, chemists need to analyse individual fractions by thin-layer chromatography, mass spectrometry or NMR after the chromatographic separation. For the ChemPU integration of the module several alternative options were considered. It was found that by consideration of the UV/vis response or the signal from the elastic light scattering detector (ELSD) of the eluting fractions and by choosing the peak with the largest area under the curve for a specified signal trace gives the best trade-off between reliability and flexibility: for a given well-performing reaction the product peak can correctly be identified independent of the exact retention time. Moreover, this method does not rely on more elaborate product identification such as mass spectrometry or NMR.

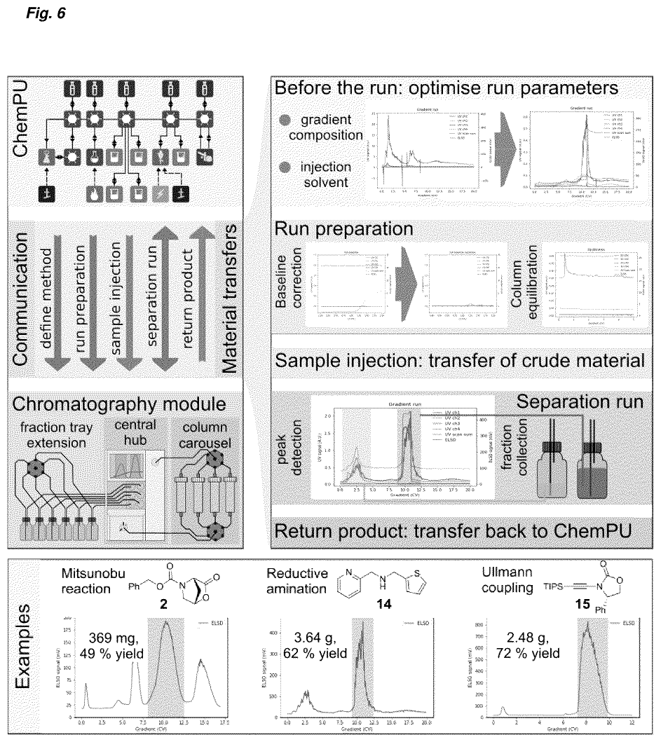

Once the method is developed and coded in _{X}DL it can be executed on the chemical synthesiser or equivalent automation system as shown in FIG. 6. The platform controller starts the chromatography process by defining the run parameters on the commercial chromatography unit (central hub), such as flowrate and detector settings. Then the actual run preparations are executed such as baseline corrections and the equilibration of the column. Next the sample of crude material is dissolved, transferred to the chromatography machine, and injected onto the column. The sample injection process also includes a rinsing sequence to minimise loss of material during the sample dissolution and transfer. Once the sample loading is complete the gradient run is started. During the gradient run the chromatography machine continuously reads the detector signals and sends them to the ChemPU controller software in real-time.

The ChemPU controller then performs the peak detection and triggers the fraction collection mechanism of the chromatography machine. The controller also keeps track of fraction vial filling levels and various run parameters such as back-pressure build-up, solvent vapour levels, and solvent levels of the gradient solvents and the solvent waste drum. If any of these parameters exceed the specific threshold an appropriate error-handling routine is initiated that pauses the chromatographic separation in a controlled way. When the separation run is complete the product peak is identified and transferred to the next module (usually the rotary evaporator). Since typically the crude material is transferred from the rotary evaporator to the chromatography module and then the purified product is transferred back from the chromatography module to the rotary evaporator, the rotary evaporator flask needs to be cleaned in between. Hence an optional cleaning routine for the target vessel of the purified product has been implemented and can be performed during the chromatographic separation. The integrated chromatographic separation was used for three reactions. The process of these chromatographic separations has been captured by _{X}DL, specifying every minute and critical detail, in a concise, easy-to-understand way. Hence, reproduction of the chromatographic separations on a Chemputer or equivalent system, or even manually with a commercially available chromatography machine, is readily possible.

### General Materials and Experimental Details

Solvents and reagents were used as received from commercial suppliers unless otherwise stated.

NMR measurements were performed with Bruker Avance III HD 600 spectrometer operating at 600.1 and 150.9 MHz for ¹H and ¹³C, respectively. Spectra were collected at 298 K, chemical shifts are reported in ppm and were calibrated for the (residual) NMR solvent signal (multiplicities are given as s: singlet, d: doublet, t: triplet, q: quartet, m: multiplet, with coupling constants reported in Hz). The spectra were processed with MestReNova 14.0.0.

HPLC-UV/Vis (MS) analysis was performed on a Thermo Dionex Ultimate 3000 equipped with an LPG-3400 RS pump, a WPS-3000TRS autosampler, a TCC-3000SD column compartment and a DAD-3000 diode array detector. 10 μL of each sample was injected on to an Agilent Porodhell 120 EC-C18 2.7 μm, 4.6×150 mm column, eluting at 1 mL/min with mobile phase A being water+0.1% formic acid and mobile phase B acetonitrile+0.1% formic acid, detecting using UV (λ=190, 214, 220 and 254 nm). The total run time was 26 minutes, with the LC method as follows—0 min—0% B, 4 min—10% B, 16 min—70% B, 19 min—100% B, 23 min—0% B, 26 min—0% B. Column compartment was set at 30° C. The Thermo Dionex Ultimate 3000 HPLC was connected to a Bruker MaXis Impact quadrupole time-of-flight mass spectrometer with an electrospray source, operating exclusively in negative mode. The instrument was regularly calibrated using Agilent ESI-L Low Concentration Tuning Mix. Samples were introduced into the MS at a dry gas temperature of 200° C. The ion polarity for all MS scans recorded was negative, with the voltage of the capillary tip set at 4500 V, end plate offset at −500 V, nebuliser at 1.6 bar, dry gas at 8.0 l/min, funnel 1 RF at 400 Vpp, funnel 2 RF at 400 Vpp, isCID energy at 0 eV, hexapole RF at 100 Vpp, ion energy 5.0 eV, low mass at 50 m/z, collision energy at 5 eV, collision cell RF at 200 Vpp, transfer time at 63.5 μs, and the pre-pulse storage time at 1.0 μs. The mass range was set to 50-2000 m/z. Data was analysed using the Bruker DataAnalysis v4.1 software suite or MestReNova 14.0.0.

GC-MS analysis was performed on Agilent 7890A GC system equipped with Agilent 5975C inert XL MSD with triple-axis detector. For the standard, non-chiral analysis the GC was equipped with an Agilent HP-5MS column (30 m, 0.250 mm diameter, 0.25 μm film) and run with a flow of 1.0 mL/min of helium. Samples were injected in splitless mode with the inlet liner temperature at 290° C. The oven temp was kept at 40° C. for 1 min and then ramped at 20° C./min to 310° C. and then held at that temperature for 7 min. The MS analysis was run with 3 min solvent delay and the mass range from 100.0 to 550.0 Da was scanned with a resolution of 0.1 D at a cycle time of 1181.86 ms.

3D-Printing was performed on a Connex 500 printer from Stratasys using the Fullcure 720 translucent resin for the major body of the printed parts and VeroBlack as the coloured resin in cases where two-coloured prints were required. Once the print was complete, the parts were cleaned first by scrapping away the bulk of the support material manually, followed by thoroughly washing them in a water jet cleaning station (Quill Vogue Polyjet). Finally, the parts were placed in a 0.1 M aq NaOH bath for 30 min and then cleaned again in the water jet cleaning station. Particular attention was paid to small holes for screws and magnets to ensure that no residual support material was present.

Laser Cutting was performed on a Monster1060 CO₂ Laser system (ML1060 130 W) from Radecal with the RDWo rksV8 software. The applied parameter sets are summarized in the Table below.

TABLE 1
Settings for the laser cutter

| Material | 6 mm acrylic | 4 mm acrylic |
|---|---|---|
| Laser power | 70%-85% | 50%-65% |

| | | | | |
|---|---|---|---|---|
| Speed | 10 | mm/s | 17 | mm/s |

| | | |
|---|---|---|
| Air | On | On |
| Laser through mode | Enabled | Enabled |

| | | | | |
|---|---|---|---|---|
| Air pressure | 0.3 | MPa | 0.3 | MPa |
| Flow rate | 33 | L/min | 33 | L/min |

### The ChemPU System

### Software Overview

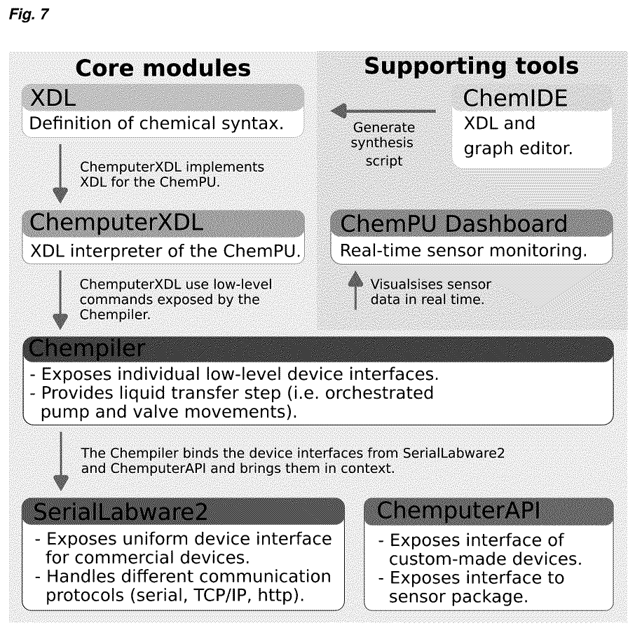

The ChemPU software stack consists of five core modules and two additional tools (FIG. 7). The modules are written in Python and are compatible with Python 3.7 to 3.9.

The XDL module defines the general syntax for chemical procedures. It is based on XML and thus lends itself for automated processing—either for execution of the protocol or at a later stage for mining of synthesis procedures—while being intuitive and human-readable. Importantly, the XDL module and XDL syntax are not specific to the ChemPU and do not depend on any other Python module shown in FIG. 7. In fact, the XDL module and syntax are entirely platform independent and serve the sole purpose of capturing and communicating chemical synthesis protocols without ambiguity and missing details.

The ChemPUXDL module inherits the XDL module, thereby generating the ChemPU-specific interpretation of XDL while obeying the rules imposed by XDL. By starting from the XDL module in the implementation of ChemPUXDL it is ensured that any synthesis protocol written to the XDL standard can be executed on the ChemPU. Likewise, a synthesis protocol written in XDL and tested on the ChemPU can be executed on any other laboratory automation system that inherits the XDL module. The ChemPUXDL module takes each synthesis step defined in the XDL module and translates it into the specific ChemPU base commands, which are exposed by the Chempiler module.

The Chempiler module has two tasks: 1) it gathers all device interfaces and brings the individual devices into context (e.g. captures physical and logical connections) and 2) it provides the logic for simple concerted tasks such as moving liquid through the liquid handling backbone.

The Chempiler pulls device interfaces from SerialLabware2 and ChemputerPUAPI which are both Python libraries containing the low-level commands and communication protocols to address the individual devices that make the ChemPU. For example, SerialLabwer2 exposes the application programming interface of the Buchi rotary evaporator R300 and the Buchi chromatography machine Pure C815. It also contains interfaces to other devices such as different hotplates, recirculating chillers and overhead stirrers. The ChemputerPUAPI module is very similar to the SerialLabware2 module in its function. The main difference is that the ChemputerPUAPI defines the interfaces for devices that were not commercially available but built in-house. It also contains a range of different sensors for reaction and process monitoring.

### Software Installation

Since the ChemPU Python software stack has a number of specific requirements to other third-party Python modules its best practice to install it in a dedicated Python environment. Importantly, to avoid frustration, the ChemPU Python modules should not be installed in the system Python path.

It is suggested to install a Python version managing system such as Miniconda (https-J/docs.conda.io/projects/conda/en/latest/user-guidefinstall/download.html, 23 Jun. 2021 at 18:58 BST). Once Miniconda or equivalent has been installed and a Python environment dedicated for the ChemPU software stack has been initialized with a Python version of either 3.7, 3.8 or 3.9 the actual ChemPU software modules can be installed.

While any XDL protocol can be executed as a simple Python script in the Miniconda terminal or equivalent, it was found to be more convenient to run the synthesis script as a Jupyter Notebook. Jupyter Notebook or Jupyter Lab can be installed via Miniconda.

### Development Process of _{X}DL Procedures

The generation of a _{X}DL procedure starts by translating the literature procedure into the _{X}DL syntax. Then the _{X}DL procedure is updated with steps that are implied but not explicitly mentioned in the literature such as Evaporate steps and rinsing sequences when material is transferred from one vessel to another. Next, steps that are necessary for the housekeeping but would not usually be considered as part of a synthesis procedure in a manual workflow are added such as ResetHandling steps to clean the liquid handling system of the platform with the appropriate solvent or CleanVessel steps to clean glassware on the fly before it is reused. Finally, some process optimisation can be done. For example, since solid additions are not currently automated, if a solid has to be added in the middle of a synthesis it may be more convenient to pre-charge it in a separate vessel and then dissolve it automatically.

With the finished _{X}DL procedure in hand, the synthesis was executed on the ChemPU. Depending on the outcome of the synthesis run the _{X}DL procedure was modified as needed. In cases where further iterations of the _{X}DL were needed, the cause of the issue fell into either of two categories. First, there are problems with the automated execution, such as a software bug or a hardware failure. Second, there may be a problem with the chemistry. For issues falling into the first category, the problem was recorded and logged as an issue on our gitlab account. It was then addressed by either the software developers or the engineering team and a bugfix or engineering change was then rolled out. Problems with the actual chemistry were addressed by changing parameters in the _{X}DL, which was usually based on further literature research to better understand the chemical process.

In average, it took approximately three iterations to generate a validated _{X}DL procedure that gives a satisfactory result starting from a literature text. While it is not directly possible to compare the yield achieved in the literature to the yield achieved with the ChemPU due to different reaction scales and minor differences in the process, we aimed to achieve a yield that was no less than 15% lower than the literature yield. When optimising the _{X}DL, the aim was to achieve sufficient purity to record NMR spectra that meet the standard for publication.

### Synthesis Protocol Execution

Most synthesis protocols reported here have been executed from a Jupyter Notebook.

### Process for New Hardware Additions

The addition of new hardware modules to the ChemPU hardware library is straight forward and greatly simplified by the modular architecture of the various software modules. There are two scenarios for hardware additions. First, an alternative hardware unit for an existing unit may be added. For example, one may want to add a hotplate stirrer from a different supplier. Second, a hardware unit with entirely new functionality may be added such as a chromatography system—which has been added in this work—or a photochemical or electrochemical module.

a. Add Alternative Hardware Modules

The device needs to be integrated in the SerialLabware library. Once the device class is there, it can be used directly with the rest of the software stack.

b. Add Hardware Modules with New Functionality

The same integration as for an alternative hardware module (see Section 0) has to be performed. Additionally, a new XDL step (e.g. Irradiate for photochemistry or ColumnChromatography for flash column chromatographic separations) has to be defined in the XDL library and implemented in the ChemPUXDL library. Additionally, the Chempiler module needs to be updated with relevant constants and wrappers.

### Hardware Installation

The ChemPU hardware has been extensively described in Angelone et al. and Fyfe et al.

### Frame and Shelving

The shelving unit provides the frame to support not only the pumps and valves for the liquid-handling backbone, but also other components. Since the fume hood dimensions vary from lab to lab the frame to support the shelve was custom-made.

The frame and shelving consist of three major components: a frame, an upper shelf for the pumps and a lower shelf for the valves. Dimensions are given to fit the shelving unit in a 1000×1700 mm×600 mm (H×L×D, internal) fume cupboard. For fume cupboards of different size, the frame dimensions might be adjusted accordingly, while the dimensions of the shelves can be kept the same. The frame was built from OpenBuilds® parts.

### Switch

The connectivity between the PC running the software and the ChemPU hardware is achieved by the PoE-capable switch. This means that the switch can provide power and Ethernet connection over the same cable. The PoE switch of preference is the Netgear GS752TPP, providing 48 ports and a maximum of 750 W of power at 48 V. No configuration is required for that switch.

### Electronics

Custom-made electronics were used to drive the pumps and valves in the liquid handling back-bone of the ChemPU. These are powered and controlled over Ethernet (PoE). The electronic assembly for a single pump or valve is composed of two boards: the controller and the stepper driver. At the heart of the controller board is a microcontroller (Atmel XMega128A4U) which is programmed with a specific firmware (see Firmware section below). The controller board is powered over PoE with a dedicated DC-DC step-down converter. The Ethernet connectivity is provided by a W5500 embedded Ethernet controller from WizNet. The controller board also carries two rows of standard 100 mil pitch pin headers along edges serving as mezzanine connectors for the stepper driver board. The stepper driver board is plugged on top of the controller board. It gets unconverted 48 V PoE voltage to supply the stepper motor as well as 5 V from the controller board to supply the stepper driver chip. The chip used is a TMC262 stepper motor controller from Trinamic, which is controlled by the main MCU via STEP/DIR interface.

### Manufacturing

The manufacturing of all the custom-made electronics was undertaken by SOUMAC Ltd. The manufacturing company was provided with Gerber files, a bill of materials and pick and place instruction files.

### Firmware

The firmware was flashed onto the control boards using an Atmel ICE mkIII (firmware version 1.27) programmer. A simple batch script invoking avrdude (an open source tool for flashing/reading Atmel AVR microcontrollers available at https://www.nongnu.org/avrdude/) was used for the actual flashing process for convenience. Alternatively, the firmware can be flashed from the Atmel Studio software package freely available from Microchip website. For the Atmel ICE mkIII programmer to work correctly both with avrdude and Atmel studio, libusb filter drivers have to be installed first.

### Configuration

After fully assembling the pumps and valves and flashing the firmware, the devices were configured and tested. For this purpose, a custom Python script was executed and the dialogue was followed. The firmware comes with default settings, which must be adjusted depending on the physical configuration of the device instance. In the case of a pump, for example, the installed syringe size must be specified. After configuration, the script automatically tests the device by executing a few simple movement commands.

### Hall Sensor Assembly

The Hall sensor board is used both in the pump and in the valve to calibrate the home position. The fully assembled sensor boards can be obtained from a chosen EMS/ECM provider. Each pump and each valve require one Hall sensor assembly. The Hall sensor assemblies for pumps and valves use a different Hall sensor board. The exact total number of assemblies required depends on the size of the liquid handling backbone that is assembled and the number of auxiliary valves. In the standard build, six Hall sensor assemblies for pumps and seven Hall sensor assemblies for valves are needed.

### Pump Assembly

The ChemPU uses custom-made pumps in the liquid handling backbone. These are controlled and powered over Ethernet. The pumps were equipped with either 5 mL, 10 mL or 25 mL syringes (as specified for each instance of the ChemPU).

The standard instructions for 3D printing with the Connex 3D printer were followed (consult the relevant manufacturers manual). The parts were cleaned according to the standard procedure.

The commercially available syringes (ILS part number: 2624093 [for 10 mL syringes], 2624076-HT [for 25 mL syringes]) may fail after long-term use in a chemical synthesiser (though durability strongly depends on what chemicals are handled). Instead of replacing broken syringes, it was found that they can be repaired rather easily, and good results were obtained (i.e. low failure rate in long-term use). For critical applications, it may even be considered to perform the following re-gluing process as a preventive maintenance.

### Valve Assembly

The chemical synthesier uses custom-made valves in the liquid handling backbone. These are controlled and powered over Ethernet.

The standard instructions for 3D printing with the Connex 3D printer are followed (consult the relevant manufacturers manual). The parts are cleaned according to the standard procedure.

The motor is assembled as described above.

### Liquid Handling Backbone

The pump and valves were connected to form the liquid handling backbone—the core of the physical instance of the ChemPU. The ChemPU architecture allows for much flexibility in terms of how the liquid handling backbone is designed. First the liquid routing is planned and visualized as a graph using a suitable graph editor (ChemIDE—https://croningroup.gitlab.io/ChemPU/xdlapp/—provides a graph editor). This helps to highlight any possible problems with liquid routing and provides a reference schematic for the following assembly. During assembly, all tubing must be cut with an appropriate tube cutter.

### Inert Gas Supply

Establishing an inert atmosphere is a key step in many synthetic transformations. Hence, inert gas supply is a critical feature of any automated synthesis platform. The ChemPU architecture provides two solutions for inert gas supply. In the first approach a constant positive pressure of a protective gas is provided, while the second approach consists of a programmable pneumatic manifold that functions analogous to a Schlenk line.

### Programmable Pneumatic Panel

The programmable manifold was designed with the typical synthetic chemists' needs in mind:

Providing positive inert gas overpressure

Providing an option to evacuate the vessel for filtration/inert gas re-fill

Providing pressure differential between arbitrary outlets to perform cannula transfer-type manipulations

To provide the above, the system was designed as a stack of two rows of six 2/3 electromagnetic valves. The frontend row (referred to as frontend valve manifold hereafter) of valves switches between vacuum (normally closed) and the output of the backend row of valves (referred to as backend valve manifold hereafter). The backend valve manifold switches between low-pressure (normally open) and the high-pressure inert gas supply. Additionally, the high-pressure inert gas supply can be manually switched between the high-pressure gas and ambient air for vacuum relief/drying purposes. When all valves are de-energized, the system provides low pressure inert gas on all 6 outputs. Excessive pressure is vented to the atmosphere through the outlet flow meter and the check valve. The valves are controlled by an Arduino board with custom-made shield hosting 12 MOSFETs and an Ethernet-to-serial converter. The programmable manifold can be supplied with up to 8 bar of inert gas.

### Reagent Bottles

Two types of reagent bottle setups were used. The standard setup allows for keeping the reagent under a positive pressure of argon if needed. In the advanced setup, the flask can additionally be chilled and stirred (that is used, for example, when a solution needs to be freshly prepared from a solid reagent just before use).

### Standard Reactor

The ChemPU used the three-neck round bottom flask as the standard reactor based on the ChemPU paradigm to mimic the manual organic chemistry workflow. The flask size was chosen according to the needs of the synthesis at hand. The instructions assume a 250 mL flask and connection to an IKA RET control-visc hotplate stirrer. Connection to an IKA RCT digital is also possible and support in the software.

The jacketed filter module is used in steps where the reaction product has to be separated as a solid such as in a re-crystallisation or a precipitation step. It can even be used for simple reactions where the product is formed as a solid. The reactor part of the module is a custom glass-blown device.

During synthesis it may be necessary to apply vacuum and/or a positive pressure of inert gas to the outlet of the jacketed filter as well as to remove liquids from the outlet via the liquid handling backbone. To be able to switch between vacuum and liquid handling backbone a dedicated valve and a Woulff-type bottle were placed between the reactor outlet and the liquid handling backbone.

### Separator

The separator module was used to perform liquid-liquid extractions. It consists of an overhead stirrer, a separating funnel, and a conductivity sensor, which detects the phase-boundary.

The phase boundary in the separating funnel is detected based on a difference in conductivity of the aqueous and organic phase by a conductivity sensor.

### Rotary Evaporator

The rotary evaporator module comprises a actual rotary evaporator with heating bath, and rotator. Additionally, it includes a separate vacuum pump and optionally a chiller to cool the condenser coil. Two types of rotary evaporators have been integrated in the ChemPU module library so far: an IKA RV10 digital rotary evaporator combined with a Vacuubrand Vario Plus vacuum pump and the modem Büchi R-300 rotary evaporator with Büchi V-300 vacuum pump and F-308 chiller.

### Chromatography Module

The commercially available C815 chromatography system was fully integrated with the liquid handling and logical control of the ChemPU.

The C815 chromatography system was running a custom Pure software version on the device itself. Remote control to the Pure software was established through the OpenPure server which was run locally. Both the custom Pure version and the OpenPure server were provided by Büchi on request together with installation instructions.

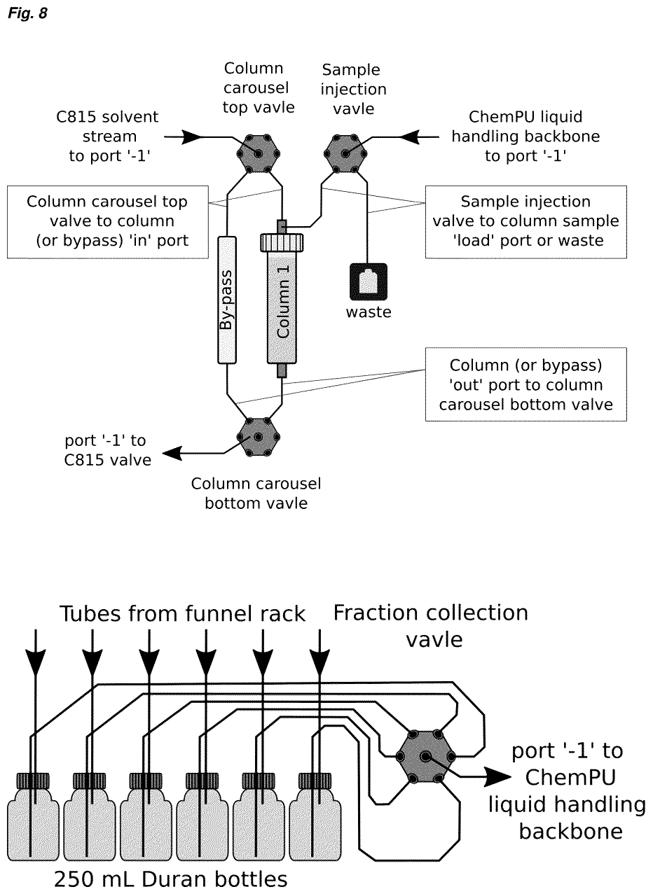

A schematic of the chromatography system and the fraction collector system is shown in FIG. 8.

### Serial-to-Ethernet Converter

One of the key incentives during the further development of the chemical synthesiser was to make the system more uniform by reducing the number of unique hardware components. One step towards this end was to include as many third-party devices as possible available over the Ethernet network, thus removing the need to have different custom interface modules. As many of the used devices had a serial port as the standard mean of communication, a serial-to-Ethernet converter was needed to make the transition from serial to Ethernet.

### N-(Pyridin-2-ylmethyl)thiophen-2-amine 14

Manual preparation

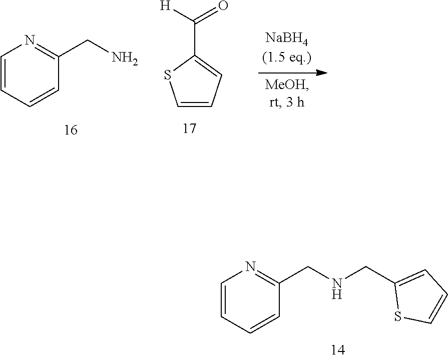 [SMILES: NCc1ccccn1 ; O=Cc1cccs1 ; c1ccc(CNCc2cccs2)nc1]

The reactor_2 was charged with NaBH4 (1.16 g, 30.0 mmol, 1.50 eq.).

### ChemPU Steps (Key Steps Only)

Add methanol (6 mL) directly to reactor_1. Add 2-thiophenecarboxaldehyde 17 (1.91 mL, 20.0 mmol, 1.0 eq.) directly to reactor_1. Add 2-picolylamine 16 (2.08 mL, 20.0 mmol, 1.0 eq.) directly to reactor_1. Heat/Chill reactor_1 to 25° C. for 60 min with stirring at 250 RPM. Transfer all from reactor_1 directly to reactor_2. Heat/Chill reactor_2 to 25° C. for 3 h with stirring at 250 RPM. Evaporate contents of reactor_2 at 45° C. for 2 h. Dissolve contents of reactor_2 in dichloromethane (50 mL) at 25° C. over 5 min, stirring at 250 RPM. Transfer all from reactor_2 directly to separator. Dissolve contents of reactor_2 in water (50 mL) at 25° C. over 5 min, stirring at 250 RPM. Transfer all from reactor_2 directly to separator. Separate mixture in separator, sending lower phase through MgSO₄ to rotavap and upper phase directly to separator. Extract contents of separator with dichloromethane (2×50 mL). Transfer waste phase (top) to waste, and product phase (bottom) through MgSO₄ to rotavap. Evaporate contents of rotavap at 50° C. for 30 min. Run preparative chromatography with DCM in petroleum ether (50% to 100% over 5 column volumes) followed by methanol in DCM (0% to 15% over 10 column volumes). Evaporate contents of rotavap at 45° C. for 30 min. Dry contents of rotavap for 30 min at 45° C.

The product N-(pyridin-2-ylmethyl)thiophen-2-amine 14 was obtained as a pale yellow oil (2.27 g, 11.1 mmol, 62%). The recorded NMR signals in methanol-d₄ are in agreement with the literature data.(43)

### Synthesis Protocols

A full synthesis definition for the ChemPU system consists of a XDL procedure, a graph file, and a short Python execution script.

The syntheses procedures have been described following the XDL standard. This task is most conveniently done in the ChemIDE web application using the step templates and the prose-to-XDL translation tool. ChemIDE also allows construction of a graph representation of the ChemPU system on which the synthesis will be executed. With a graph definition of the available hardware at hand, the XDL procedure can be translated into an executable “.xdlexe” file. This “.xdlexe” file is eventually executed with the help of a short Python script.

### Validated Synthesis Protocols

The reaction schemes for the validated XDLs are shown in the Schemes below. The code below each product structure corresponds to the name of the sub-folder in the supporting ‘XDL files’ folder containing all files necessary to execute the reaction. All multistep reaction sequences are shown together. The reactions were classified according to the reaction classes shown in the summary of reactions below. The distribution of reaction scales is shown in the graph that follows.

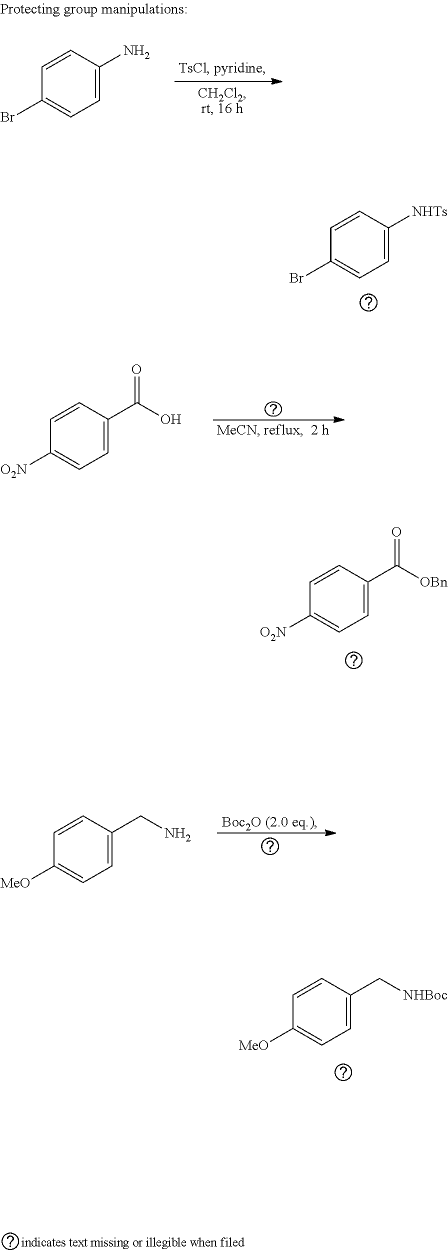

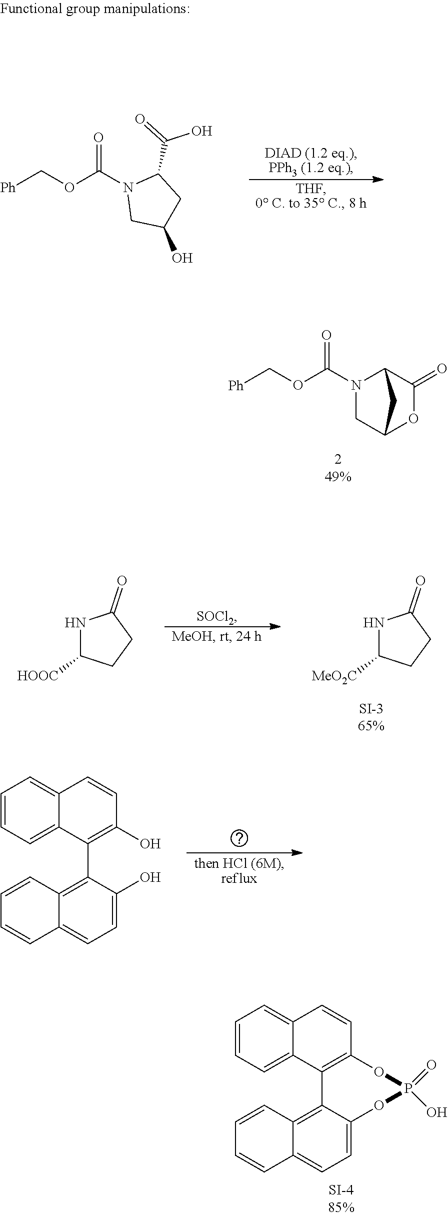

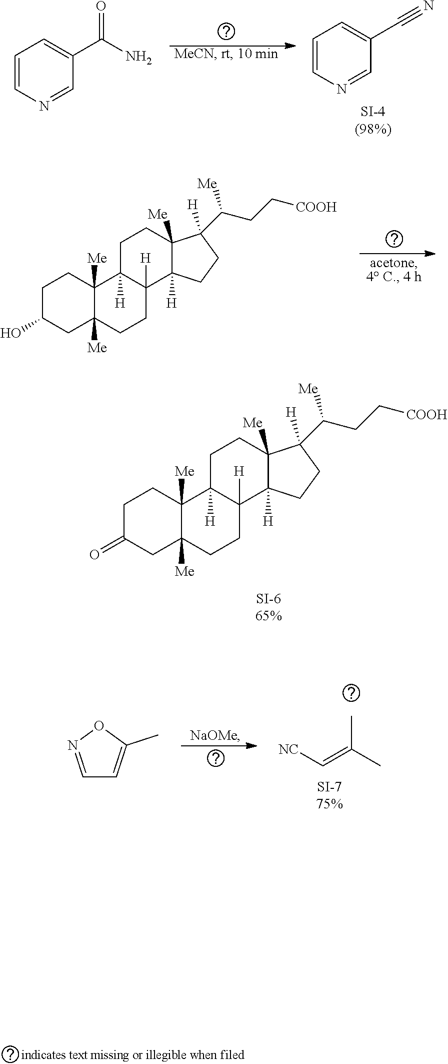

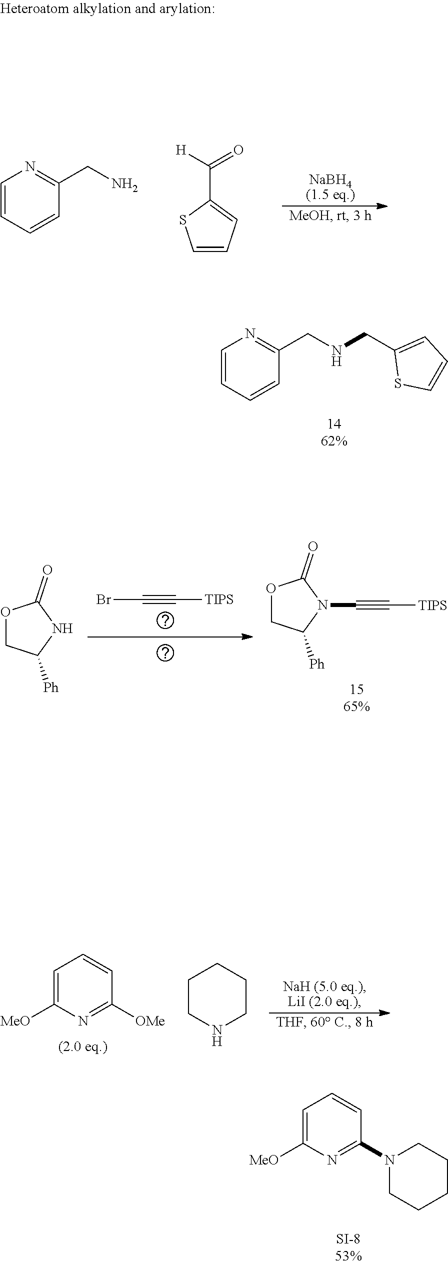

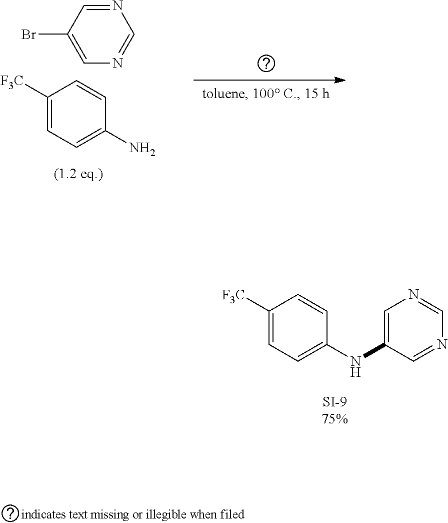

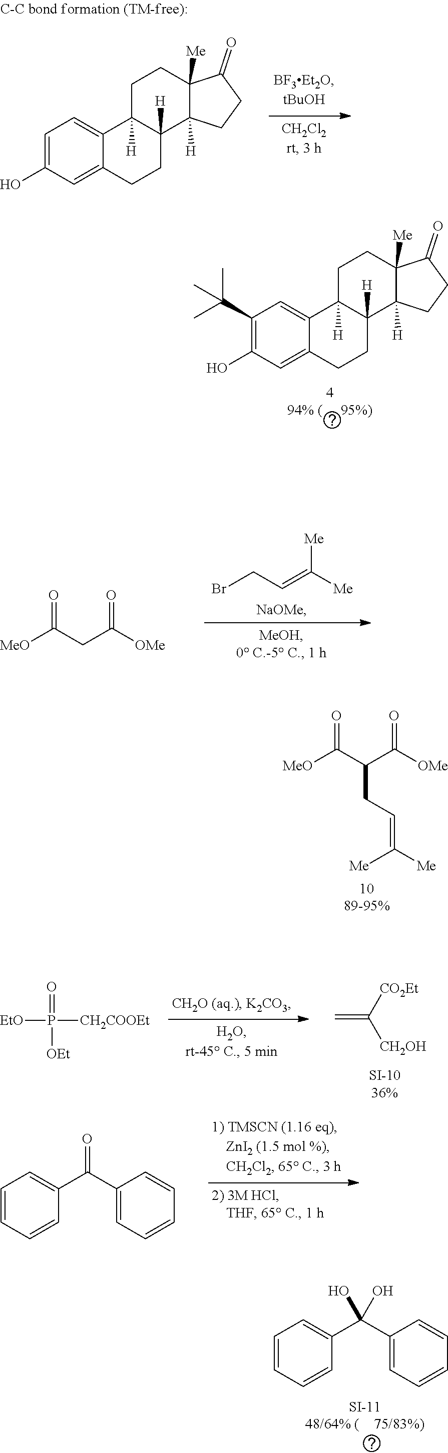

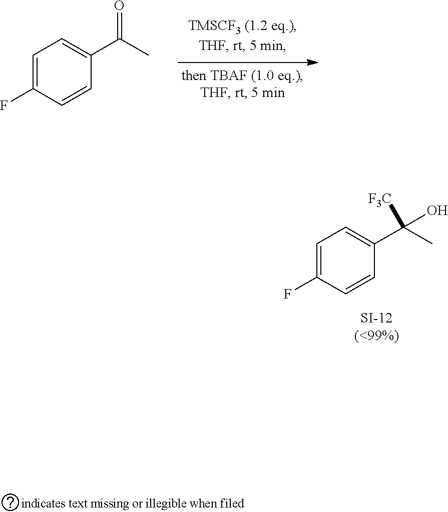

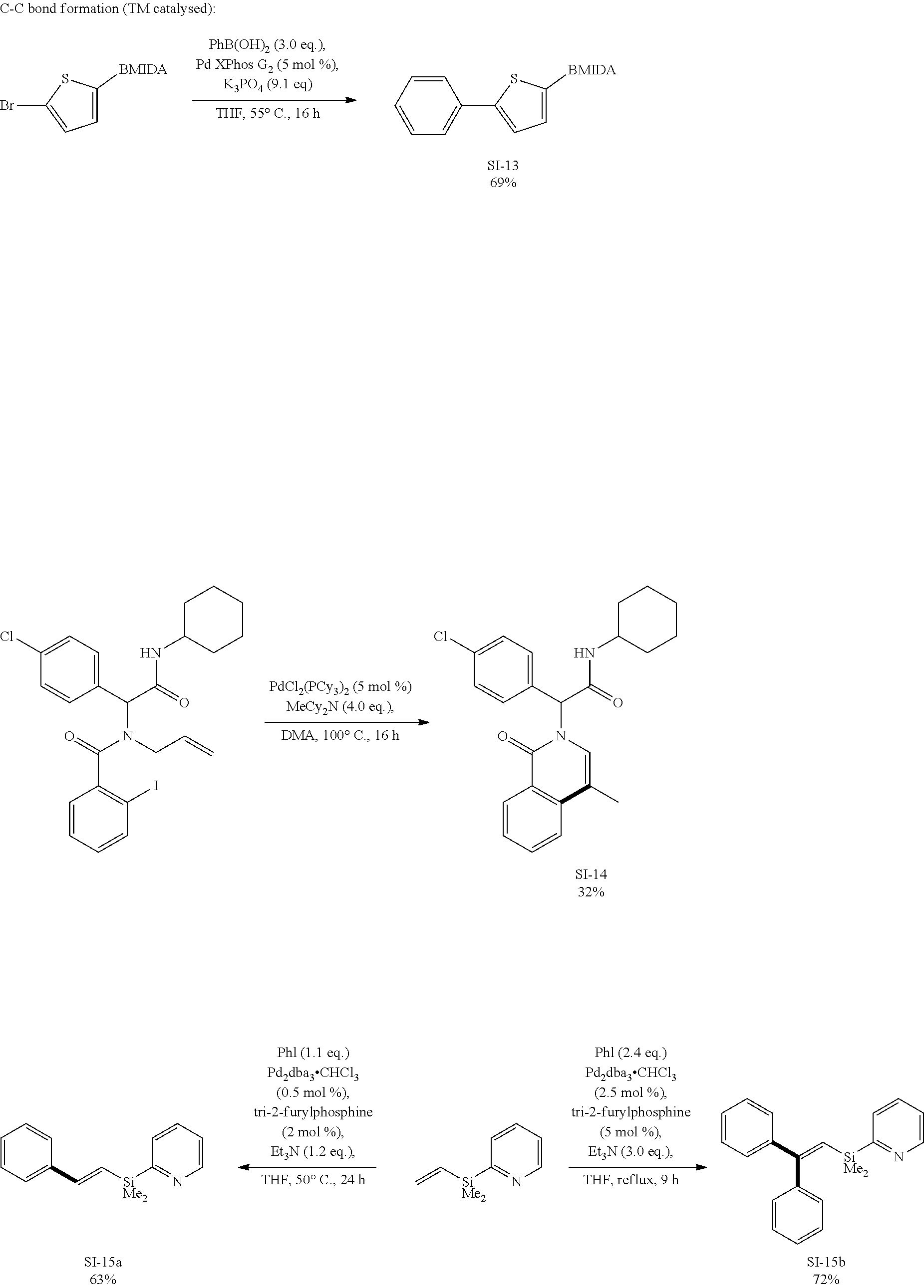

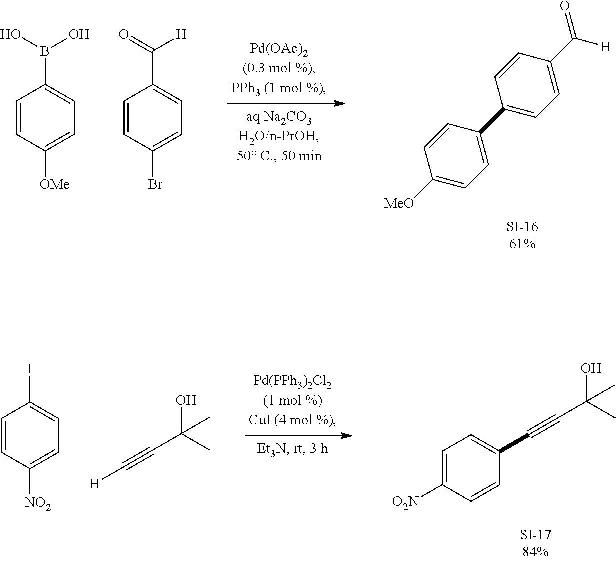

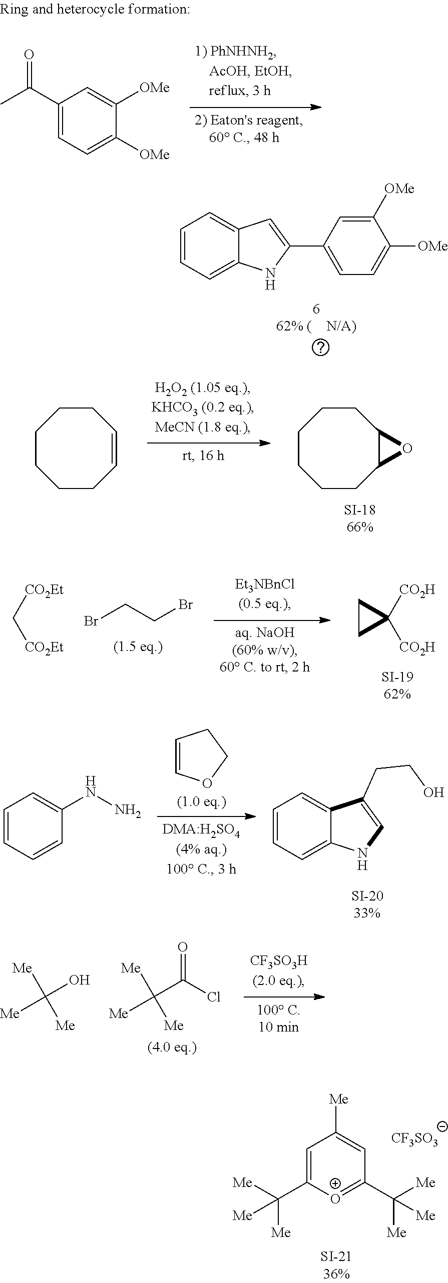

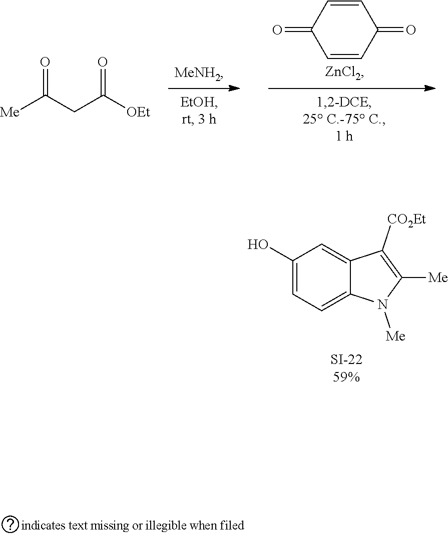 [SMILES: O=C1C=CC(=O)C=C1 ; CCOC(=O)CC(C)=O ; CCOC(=O)c1c(C)n(C)c2ccc(O)cc12]

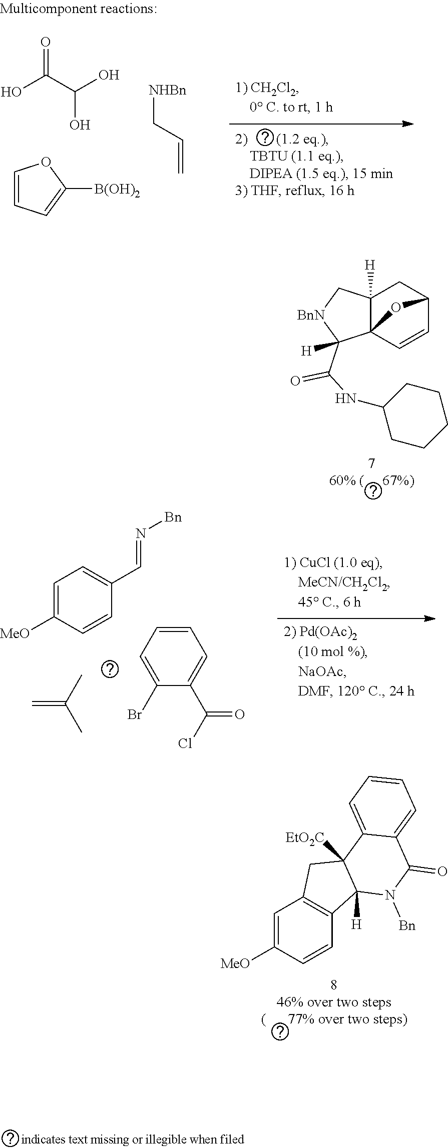

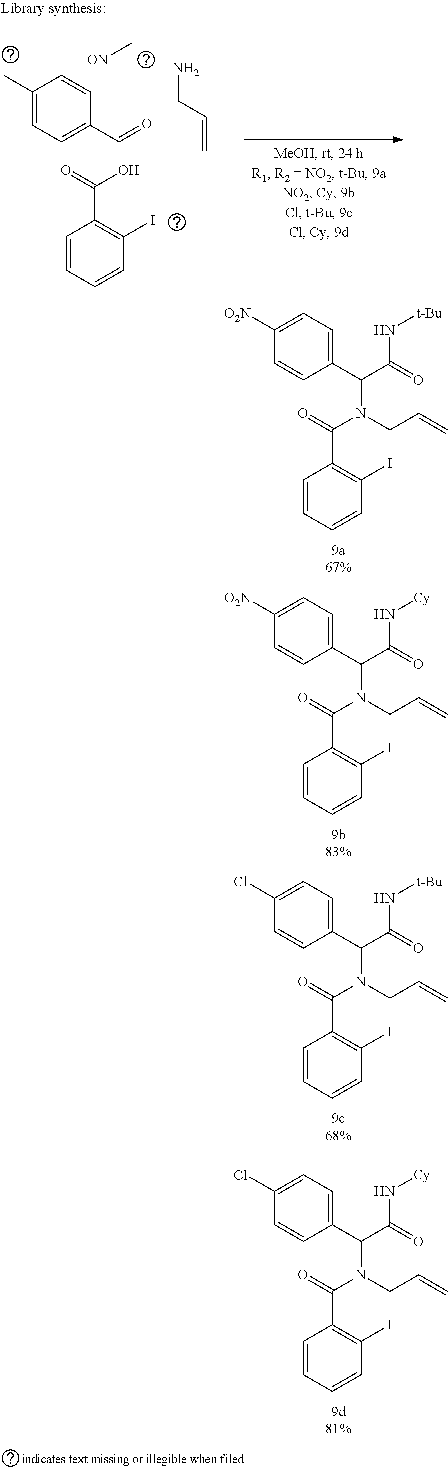

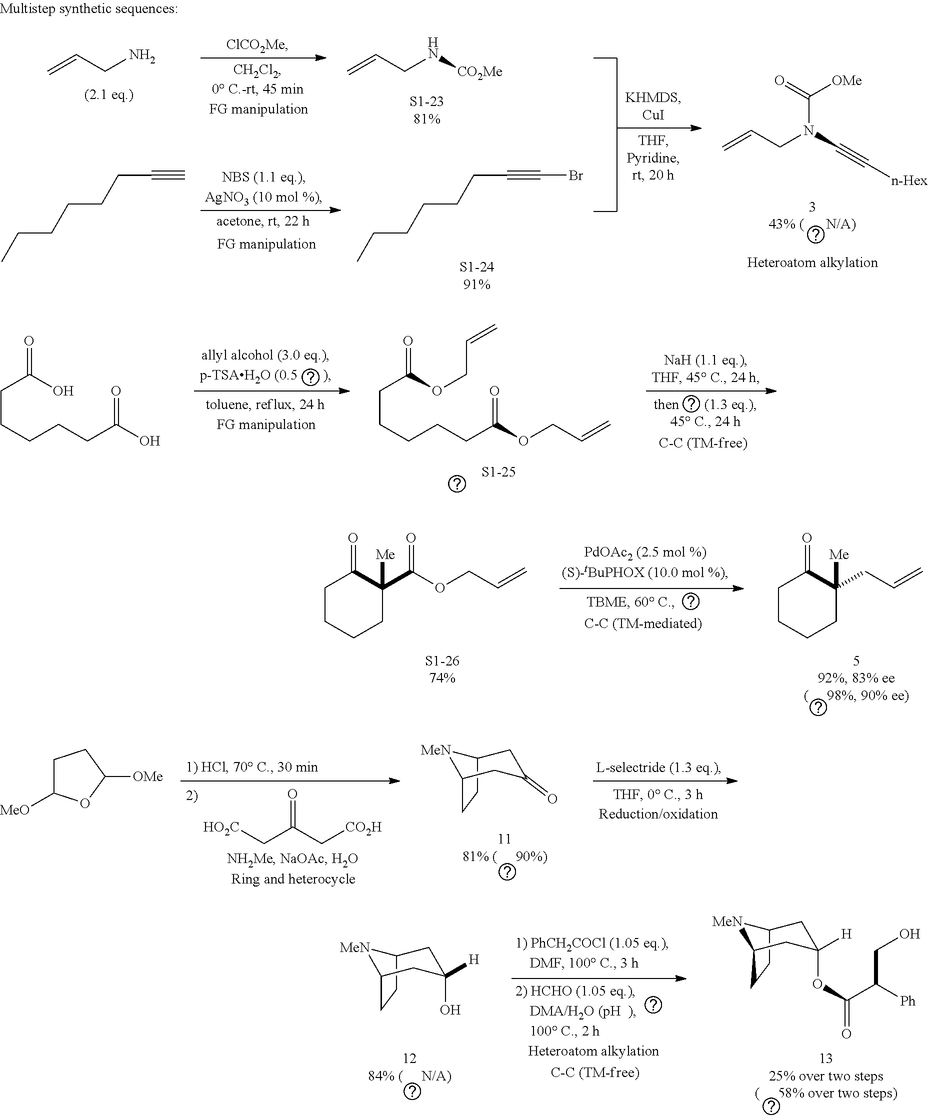

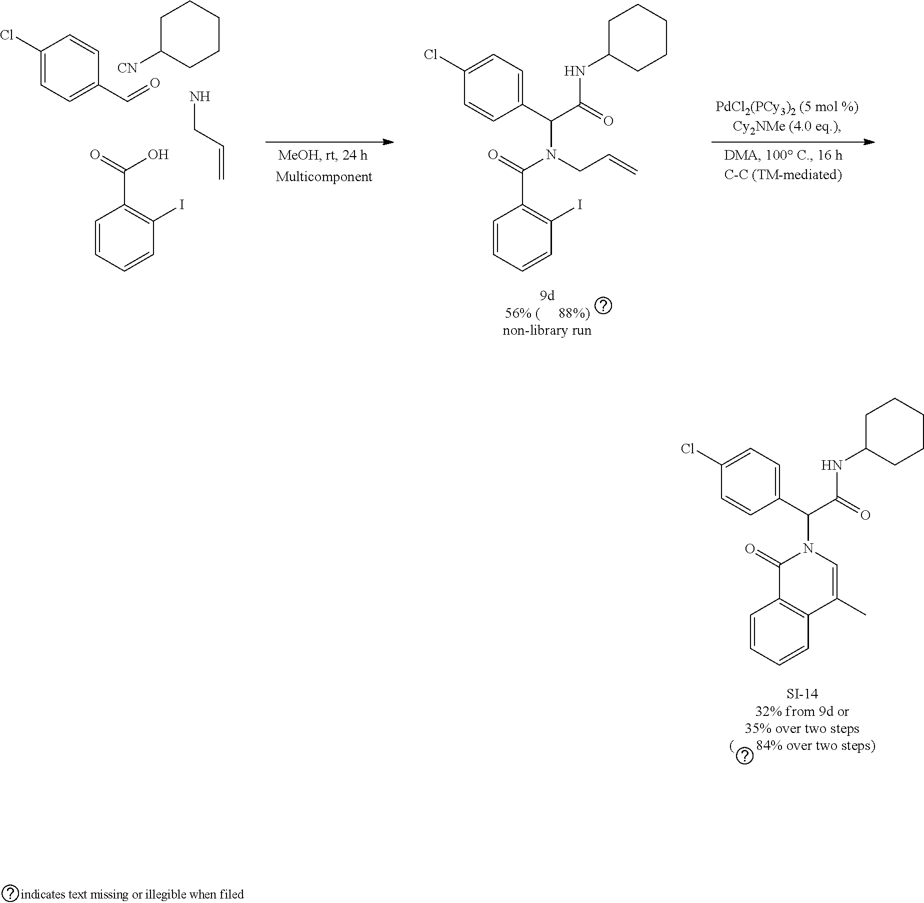

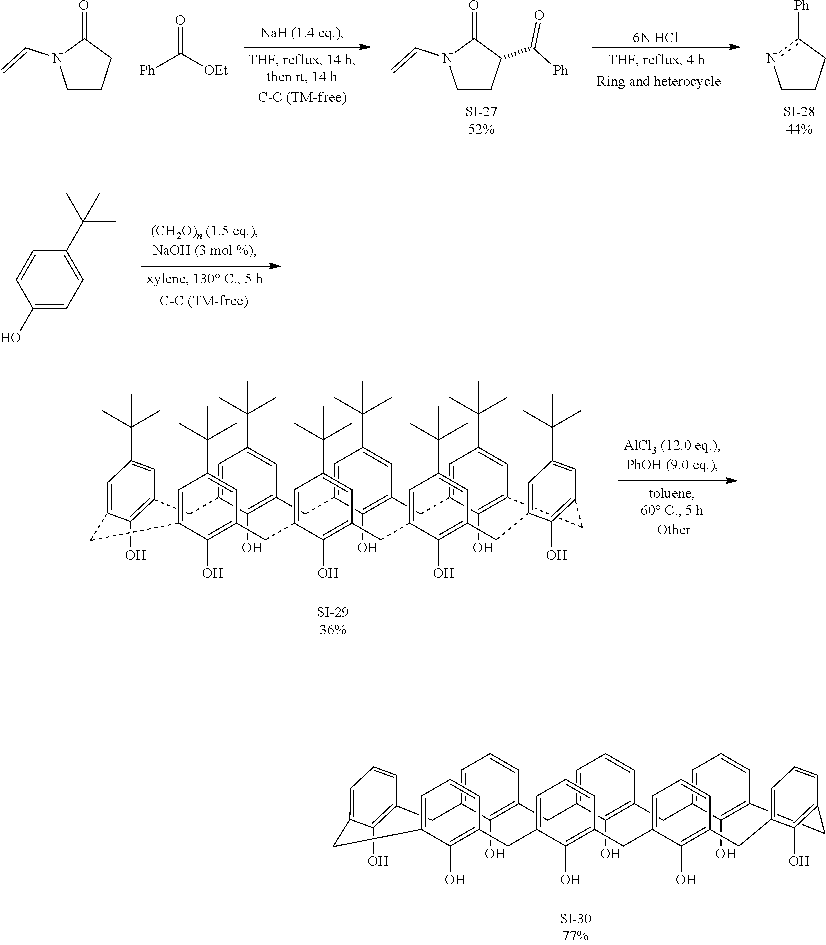

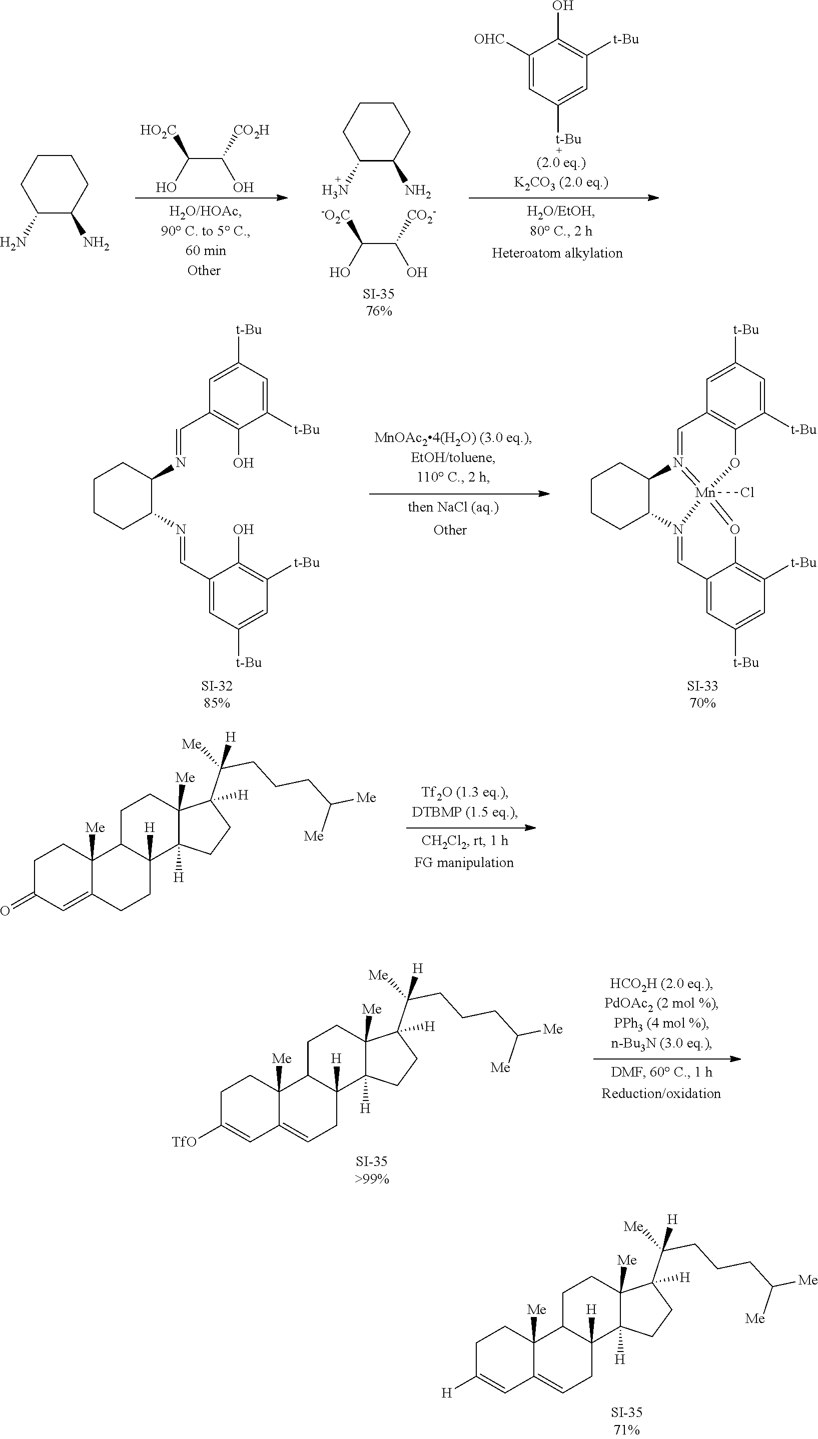

The reaction scale distribution is show below

### N-(4-Bromophenyl)-4-methylbenzenesulfonamide 1

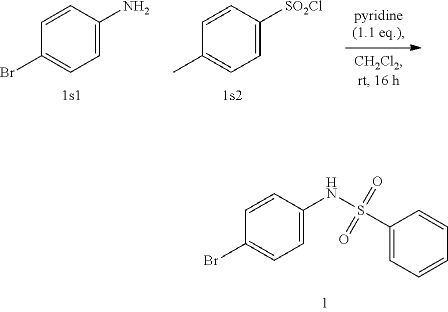 [SMILES: Nc1ccc(Br)cc1 ; Cc1ccc(S(=O)(=O)Cl)cc1 ; O=S(=O)(Nc1ccc(Br)cc1)c1ccccc1]

The synthesis was performed following literature protocol by Weix et al. (3)

### Manual Preparation

The system was configured as specified in the graph file for this reaction. The reactor was charged with 4-bromoaniline (1.51 g, 8.8 mmol, 1.00 eq.) and p-toluenesulfonyl chloride (1.71 g, 8.9 mmol, 1.02 eq.). The solution of pyridine (0.32M) in DCM was prepared manually.

### ChemPU Steps

1) Add dichloromethane (23 mL) directly to reactor at default speed without stirring.
2) Add pyridine in dichloromethane (30 mL, 0.32 M, 0.75 mL, 9.7 mmol, 1.11 eq.) directly to reactor at default speed with stirring at 250 RPM.
3) Stir reactor for 16 h at 250 RPM stopping stirring afterwards.
4) Transfer all from reactor directly to separator at default speed, without flushing tubing after the transfer.
5) Wash contents of separator with sat. NH₄Cl (aq) (1×80 mL). Transfer waste phase (top) to waste, and product phase (bottom) through undefined to separator.
6) Wash contents of separator with dichloromethane (2×20 mL). Transfer waste phase (top) to waste, and product phase (bottom) directly to separator.
7) Wash contents of separator with water (1×40 mL). Transfer waste phase (top) to waste, and product phase (bottom) directly to separator.
8) Wash contents of separator with brine (1×40 mL). Transfer waste phase (top) to waste, and product phase (bottom) through cartridge of anhydrous MgSO₄ to rotavap.
9) Evaporate contents of rotavap with pressure 98 mbar at temperature 30° C. for 30 min
10) Shut down the platform.

The product, N-(4-Bromophenyl)-4-methylbenzenesulfonamide (2.78 g, 8.6 mmol, 98%) was obtained as an off-white solid. The recorded NMR signals are in agreement with the literature data. (3)

### Phenylmethyl (1S,4S)-3-oxo-2-oxa-5-azabicyclo[2.2.1]heptane-5-carboxylate 2

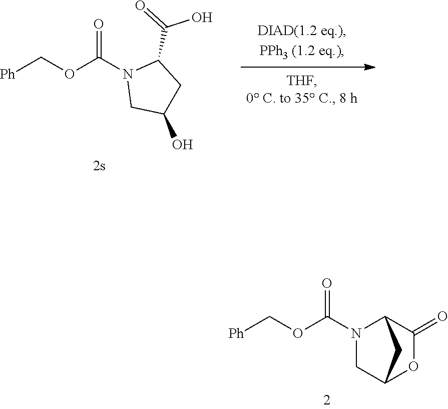 [SMILES: O=C(O)[C@@H]1C[C@@H](O)CN1C(=O)OCc1ccccc1 ; O=C1O[C@H]2C[C@@H]1N(C(=O)OCc1ccccc1)C2]

The synthesis was performed according to the modified literature protocol by Chen et al. (4)

### Manual Preparation

The reactor was charged with 1-(benzyloxycarbonyl)-4-hydroxy-L-proline 2s (796 mg, 3.00 mmol, 1.0 eq.) and PPh₃ (942 mg, 3.59 mmol, 1.20 eq.). The flask ‘DIAD_vial’ was charged with DIAD (726 mg, 3.59 mmol, 1.20 eq.).

ChemPU steps
1) Evacuate reactor and refill with inert gas 3 times, using a vacuum pressure of 50 mbar, waiting 60 s after evacuating and 60 s after refilling with inert gas.
2) Dissolve contents of reactor in THF (9.61 mL) at 25° C. over 60 s, stirring at 400 RPM.
3) Add THF (5.14 mL) directly to DIAD_vial at default speed without stirring. 4) Heat/Chill reactor to 0° C. with stirring at 250 RPM. Temperature control is continued after the temperature has been reached.
5) Transfer 8 mL from DIAD_vial directly to reactor over 15 min, without flushing tubing after the transfer.
6) Heat/Chill reactor to 35° C. for 8 h with stirring at 250 RPM.
7) Evaporate contents of reactor with pressure 50 mbar at temperature 45° C. for 60 min.
8) Dissolve contents of reactor in diethyl ether (20 mL) at 25° C. over 5 min, stirring at 400 RPM.
9) Transfer all from reactor directly to rotavap at default speed, without flushing tubing after the transfer.
10) Evaporate contents of rotavap with default pressure control at temperature 25° C. for 30 min.
11) Dry contents of rotavap for 30 min at default pressure without temperature control stopping heating when step finishes.
12) Add toluene (10.3 mL) directly to rotavap at default speed without stirring.
13) Dissolve contents of rotavap in ethyl acetate (2.57 mL) at 30° C. over 5 min, stirring at 400 RPM.
14) Run preparative chromatography with ethyl acetate in petroleum ether (10% to 60% over 12 column volumes).
15) Evaporate contents of rotavap with default pressure control at temperature 45° C. for 30 min.
16) Evaporate contents of rotavap with default pressure control at temperature 45° C. for 30 min.
17) Dry contents of rotavap for 30 min at default pressure at temperature 45° C. stopping heating when step finishes.

The product phenylmethyl (1S,4S)-3-oxo-2-oxa-5-azabicyclo[2.2.1]heptane-5-carboxylate 2 was obtained as a white solid (366 mg, 1.48 mmol, 49% yield). The NMR signals recorded in chloroform-d at room temperature are in agreement with the literature data. (4)

### Methyl Allylcarbamate—SI-23

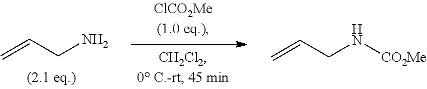

The synthesis was performed following the literature protocol Danheiser et al. (5)

### Manual Preparations

The system was configured as specified in the graph file. A solution of 9 mL methyl chloroformate in 35.5 mL CH₂Cl₂ was prepared.

### ChemPU Steps

1) Add allylamine (14.0 mL, 187 mmol, 2.1 eq.) directly to reactor at default speed without stirring.
2) Add dichloromethane (100 mL) directly to reactor at default speed with stirring at 250 RPM.
3) Heat/Chill reactor to 0° C. with stirring at 250 RPM, continuing temperature control after the temperature has been reached.
4) Add methyl chloroformate dichloromethane solution (37.0 mL, 90.5 mmol, 1.0 eq.) directly to reactor over 10 mins with stirring at 250 RPM.
5) Allow to warm/cool reactor to 18.0° C. with stirring at 250 RPM, stopping temperature control once the temperature has been reached. (Note: this step took about 45 minutes and that was the time of the reaction).
6) Transfer all from reactor directly to separator at default speed, without flushing tubing after the transfer.
7) Add dichloromethane (20 mL) directly to reactor at default speed with stirring at 250 RPM.
8) Transfer all from reactor directly to separator at default speed, without flushing tubing after the transfer.
9) Add dichloromethane (20 mL) directly to reactor at default speed with stirring at 250 RPM.
10) Transfer all from reactor directly to separator at default speed, without flushing tubing after the transfer.
11) Wash contents of separator with aqueous 1M HCl solution (1×50 mL). Transfer waste phase (top) to waste and product phase (bottom) directly to separator.
12) Wash contents of separator with saturated aqueous NaHCO₃ solution (1×50 mL). Transfer waste phase (top) to waste, and product phase (bottom) directly to separator.
13) Wash contents of separator with saturated aqueous NaCl solution (1×50 mL). Transfer waste phase (top) to waste, and product phase (bottom) through MgSO₄ to rotavap.
14) Evaporate contents of rotavap with pressure 21 mbar at temperature 40° C. for 30 mins.

The product methyl allyl carbamate SI-23 (10 g, 86.5 mmol, 81%) was obtained as colourless oil. The recorded NMR signals are in agreement with the literature data. (5)

### 1-Bromooct-1-yne—SI-24

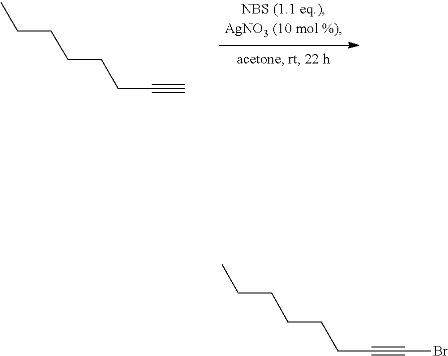

The synthesis was performed following the literature protocol by Danheiser et al. (5)

### Manual Preparations

The system was configured as specified in the graph file. The reactor flask was charged with 1-Octyne SI-24s (5 g, 45.3 mmol, 1.0 eq.) before being wrapped with aluminium foil. The rotavap flask was also wrapped with aluminium foil. NBS (8.88 g, 49.9 mmol, 1.1 eq.) and AgNO₃ (771 mg, 4.5 mmol, 10 mol %) were added manually in the corresponding steps.

ChemPU steps
1) Add acetone (107 mL) directly to reactor at default speed without stirring.
2) Add N-Bromosuccinimide (8.88 g, 49.9 mmol, 1.1 eq.) to reactor with stirring.
3) Add silver nitrate (0.770 g, 4.5 mmol, 10 mol %) to reactor with stirring.
4) Heat/Chill reactor to 25° C. for 22 hrs with stirring at 250 RPM.
5) Transfer all from reactor directly to separator at default speed, flushing tubing after the transfer.
6) Add pentanes (25 mL) directly to reactor at default speed with stirring at 250 RPM.
7) Transfer all from reactor directly to separator at default speed, flushing tubing after the transfer.
8) Add pentanes (25 mL) directly to reactor at default speed with stirring at 250 RPM.
9) Transfer all from reactor directly to separator at default speed, flushing tubing after the transfer.
10) Wash contents of separator with a saturated sodium thiosulfate solution (2×50 mL). Transfer waste phase (bottom) to vessel buffer_flask2, and product phase (top) directly to buffer_flask1.
11) Transfer all from buffer_flask2 directly to separator at default speed, without flushing tubing after the transfer.
12) Extract contents of separator with pentanes (2×50 mL). Transfer waste phase (bottom) to waste, and product phase (top) directly to buffer_flask1.
13) Transfer all from buffer_flask1 directly to separator at default speed, flushing tubing after the transfer.
14) Wash contents of separator with saturated NaCl solution (1×50 mL). Transfer waste phase (bottom) to waste, and product phase (top) through MgSO₄ to rotavap.
15) Evaporate contents of rotavap with pressure 21.3 mbar at temperature 25° C. for 30 mins.

The product 1-bromooct-1-yne SI-24 (8.58 g, 45.4 mmol, 91%) was obtained as a red oil. The recorded NMR signals are in agreement with the literature data. (6)

### Methyl allyl(oct-1-yn-1-yl)carbamate 3

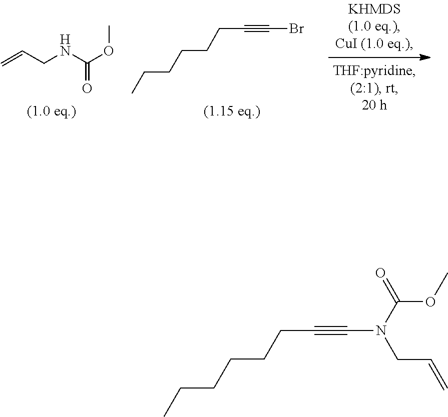

The synthesis was performed following the literature protocol by Danheiser et al. (5)

### Manual Preparations

The system was configured as specified in the graph file for this reaction. The reactor flask and rotavap flask were wrapped in foil and the reaction was performed in the dark.

The reagent flask ‘carbamate’ was charged with a weighed amount of methyl allylcarbamate 3s (1.15 g, 10 mmol, 1.0 eq.) and reagent flask ‘bromoalkyne’ was charged with 1-bromo-octyne SI-24 (2.17 g, 11.5 mmol, 1.15 eq.).

Copper (1) iodide (1.91 g) was added manually in the corresponding step.

### ChemPU Steps

1) Repeat 3 times:
   a) Add tetrahydrofuran (5 mL) directly to carbamate at default speed without stirring.
   b) Transfer all from carbamate directly to reactor at default speed, without flushing tubing after the transfer.
2) Add tetrahydrofuran (25 mL) directly to reactor at default speed with stirring at 250 RPM.
3) Add pyridine (20 mL) directly to reactor at default speed with stirring at 250 RPM. 4) Add KHMDS (11.5 mL, 0.91M in THF, 10.5 mmol, 1.05 eq.) directly to reactor over 10 min with stirring at 250 RPM.
5) Stir reactor for 15 min at 250 RPM stopping stirring afterwards.
6) Add copper (I) iodide (1.91 g, 10.0 mmol, 1.0 eq.) to reactor.
7) Stir reactor for 2 h at 250 RPM stopping stirring afterwards.
8) Add tetrahydrofuran (8 mL) directly to bromoalkyne at default speed without stirring.
9) Transfer all from bromoalkyne directly to reactor at default speed, without flushing tubing after the transfer.
10) Add tetrahydrofuran (5 mL) directly to bromoalkyne at default speed without stirring.
11) Transfer all from bromoalkyne directly to reactor at default speed, flushing tubing after the transfer.
12) Stir reactor for 20 h at 250 RPM stopping stirring afterwards.
13) Repeat 3 times:
   a) Add diethylether (20 mL) directly to reactor at default speed with stirring at 250 RPM.
   b) Stop stirring reactor.
   c) Transfer all from reactor directly to separator at default speed, without flushing tubing after the transfer.
14) Wash contents of separator with 3:1 NaCl:NH₄OH (4×30 mL). Transfer waste phase (bottom) to vessel buffer_flask. and product phase (top) directly to buffer_flask2.
15) Extract contents of buffer_flask with diethylether (2×30 mL). Transfer waste phase (bottom) to waste, and product phase (top) directly to buffer_flask2.
16) Wash contents of buffer_flask2 with 1M HCl (2×50 mL). Transfer waste phase (bottom) to waste, and product phase (top) directly to separator.
17) Wash contents of separator with sat. NaCl (1×50 mL). Transfer waste phase (bottom) to waste, and product phase (top) directly to separator.
18) Transfer all from separator through MgSO₄ to rotavap at default speed, without flushing tubing after the transfer.
19) Transfer 30 mL from separator through MgSO₄ to rotavap at default speed, without flushing tubing after the transfer.
20) Evaporate contents of rotavap with pressure 50 mbar at temperature 40° C. for 2 h.

The crude product was purified by column chromatography (0 to 5% EtOAc/pet. ether).

The product methyl allyl(oct-1-yn-1-yl)carbamate 3 (966 mg, 4.33 mmol, 43%) was obtained as a red oil.

¹H NMR (600 MHz, Chloroform-d) δ 5.85 (ddt, J=16.5, 10.3, 6.0 Hz, 1H), 5.37-5.17 (m, 2H), 4.02 (d, J=6.0 Hz, 2H), 3.78 (s, 3H), 2.27 (t, J=7.1 Hz, 2H), 1.53-1.44 (m, 2H), 1.41-1.34 (m, 2H), 1.33-1.22 (m, 4H), 0.88 (t, J=7.0 Hz, 3H). ¹³C NMR (151 MHz, Chloroform-d) δ 171.4, 134.7, 116.2, 83.0, 60.6, 41.0, 24.0, 21.2, 21.0, 17.7, 17.5, 14.8, 14.4.

### (8R,9S,13S,14S)-2-(tert-Butyl)-3-hydroxy-13-methyl-6,7,8,9,11,12,13,14,15,16-decahydro-17H-cyclopenta[a]phenanthren-17-one 4

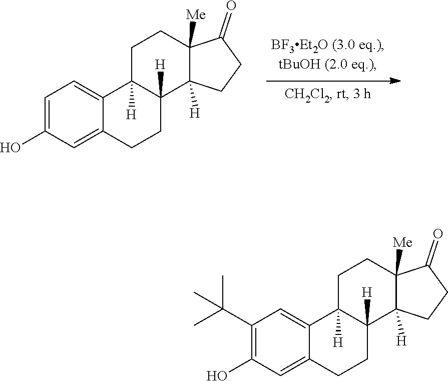

The synthesis was performed following the literature protocol by Taylor et al. (7)

### Manual Preparations

The system was configured as specified in the graph file for this reaction. The reagent flask ‘tbuoh’ was charged with a weighed amount of tert-butanol (740 mg, 10.0 mmol, 2.0 eq.).

Estrone (1.35 g, 5.00 mmol, 1.0 eq.) was added manually in the corresponding step.

### ChemPU Steps

1) Add estrone (1.35 g, 5.00 mmol, 1.0 eq.) to reactor.
2) Repeat 2 times:
   a) Add CH₂Cl₂ (5 mL) directly to tbuoh at default speed without stirring.
   b) Transfer all from tbuoh directly to reactor at default speed, without flushing tubing after the transfer.
3) Add CH₂Cl₂ (45 mL) directly to reactor at default speed without stirring.
4) Add BF₃—Et₂O (1.9 mL) directly to reactor over 30 min with stirring at 250 RPM.
5) Add CH₂Cl₂ (5 mL) directly to reactor at default speed without stirring.
6) Stir reactor for 2 h at 250 RPM stopping stirring afterwards.
7) Transfer all from reactor directly to separator at default speed, without flushing tubing after the transfer.
8) Repeat 2 times:
   a) Add CH₂Cl₂ (10 mL) directly to reactor at default speed with stirring at 250 RPM.
   b) Add Na₂CO₃ (20 mL) directly to reactor at default speed with stirring at 250 RPM.
   c) Stop stirring reactor.
   d) Transfer all from reactor directly to separator at default speed, without flushing tubing after the transfer.
9) Extract contents of separator with CH₂Cl₂ (1×30 mL). Transfer waste phase (top) to waste, and product phase (bottom) directly to separator.
10) Wash contents of separator with water (1×30 mL). Transfer waste phase (top) to waste, and product phase (bottom) directly to separator.
11) Wash contents of separator with NaCl (1×30 mL). Transfer waste phase (top) to waste, and product phase (bottom) directly to separator.
12) Transfer all from separator through MgSO₄ to rotavap at default speed, flushing tubing after the transfer.
13) Evaporate contents of rotavap with pressure 50 mbar at temperature 40° C. for 60 min.

The product (8R,9S,13S,14S)-2-(tert-butyl)-3-hydroxy-13-methyl-6,7,8,9,11,12,13,14,15,16-decahydro-17H-cyclopenta[a]phenanthren-17-one 4 (1.53 g, 4.69 mmol, 94%) was obtained as a white solid. The recorded NMR signals are in agreement with the literature data. (7)

Diallyl heptanedioate SI-25

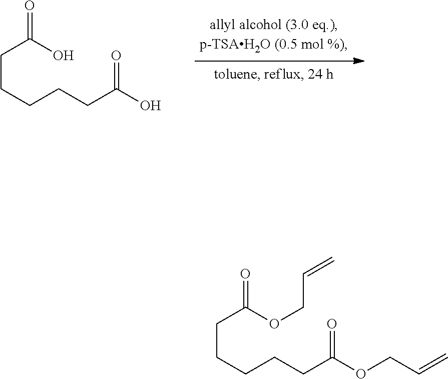 [SMILES: O=C(O)CCCCCC(=O)O ; C=CCOC(=O)CCCCCC(=O)OCC=C]

The synthesis was performed following the literature protocol by Stoltz et al. (8)

### Manual Preparations

The system was configured as specified in the graph file for this reaction.

Pimelic acid SI-25s (9.60 g, 60.0 mmol, 1.0 eq.) and p-toluenesulfonic acid monohydrate (57.0 mg, 0.03 mmol, 0.5 mol %) were added manually in the corresponding steps.

### ChemPU Steps

1) Add pimelic acid (9.6 g, 60.0 mmol, 1.0 eq.) to reactor.
2) Add pTSA (57.0 mg, 0.03 mmol, 0.5 mol %) to reactor.
3) Add toluene (30 mL) directly to reactor at default speed without stirring.
4) Add allyl alcohol (12.3 mL, 180 mmol, 3.0 eq.) directly to reactor at default speed with stirring at 250 RPM.
5) Heat/Chill reactor to 115° C. for 24 h with stirring at 250 RPM.
6) Wait for 15 min.
7) Repeat 2 times:
   a) Transfer all from reactor directly to separator at default speed, flushing tubing after the transfer.
   b) Add EtOAc (40 mL) directly to reactor at default speed with stirring at 500 RPM.
   c) Stop stirring reactor.
8) Transfer all from reactor directly to separator at default speed, without flushing tubing after the transfer.
9) Wash contents of separator with NaHCO₃ (3×30 mL). Transfer waste phase (bottom) to waste, and product phase (top) directly to separator.
10) Wash contents of separator with NaCl (2×30 mL). Transfer waste phase (bottom) to waste, and product phase (top) directly to separator.
11) Transfer 150 mL from separator through MgSO₄ to rotavap at default speed, without flushing tubing after the transfer.
12) Transfer 20 mL from separator through MgSO₄ to rotavap at default speed, without flushing tubing after the transfer.
13) Evaporate contents of rotavap with pressure 20 mbar at temperature 50° C. for 1.5 h.
14) Dry contents of rotavap for 60 min at default pressure at temperature 50° C. stopping heating when step finishes.

The product diallyl heptanedioate SI-25 (11.8 g, 49.3 mmol, 82%) was obtained as a pale yellow oil. The recorded NMR signals are in agreement with the literature data. (8)

### Allyl 1-methyl-2-oxocyclohexane-1-carboxylate SI-26

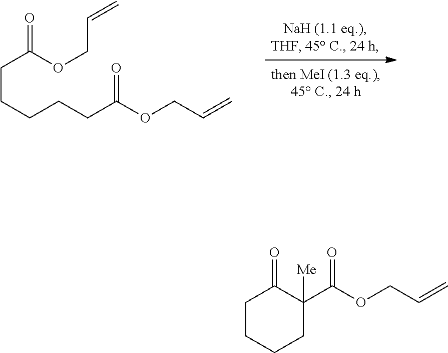 [SMILES: C=CCOC(=O)CCCCCC(=O)OCC=C ; C=CCOC(=O)C1(C)CCCCC1=O]

The synthesis was performed following the literature protocol by Stoltz et al. (8)

### Manual Preparations

The system was configured as specified in the graph file for this reaction. The corresponding reagent flask ‘diallyl pimelate’ was charged with a weighed amount of diallylpimelate (2.16 g, 9.00 mmol, 1.0 eq.). Sodium hydride (0.396 g, 60% wt., 9.90 mmol, 1.1 eq.) and iodomethane (0.73 mL, 12.0 mmol, 1.3 eq.) were added manually in the corresponding steps.

### ChemPU Steps

1) Add sodium hydride (0.396 g, 60% wt., 9.90 mmol, 1.1 eq.) to reactor.
2) Add tetrahydrofuran (2 mL) directly to reactor at default speed with stirring at 250 RPM.
3) Repeat 2 times:
   a) Add tetrahydrofuran (2 mL) directly to diallyl pimelate at default speed without stirring.
   b) Transfer 7 mL from diallyl pimelate directly to reactor at default speed, flushing tubing after the transfer.
4) Heat/Chill reactor to 45° C. with stirring at 250 RPM. Temperature control is continued after the temperature has been reached.
5) Stir reactor for 24 h at 250 RPM stopping stirring afterwards.
6) Confirm the following before continuing: Add iodomethane (0.73 mL). 7) Stir reactor for 24 h at 250 RPM stopping stirring afterwards.
8) Stop heating/chilling reactor.
9) Add water (10 mL) directly to reactor at default speed with stirring at 250 RPM.
10) Stir reactor for 10 min at 250 RPM stopping stirring afterwards.
11) Transfer all from reactor directly to rotavap at default speed, flushing tubing after the transfer.
12) Repeat 2 times:
   a) Add EtOAc (20 mL) directly to reactor at default speed with stirring at 350 RPM.
   b) Add water (20 mL) directly to reactor at default speed with stirring at 350 RPM.
   c) Transfer all from reactor directly to separator at default speed, flushing tubing after the transfer.
13) Stop stirring reactor.
14) Evaporate contents of rotavap with pressure 200 mbar at temperature 40° C. for 30 min.
15) Repeat 2 times:
   a) Add water (20 mL) directly to rotavap at default speed with stirring at 200 RPM.
   b) Add EtOAc (20 mL) directly to rotavap at default speed with stirring at 200 RPM.
   c) Transfer all from rotavap directly to separator at default speed, without flushing tubing after the transfer.
16) Clean rotavap with acetone (2× solvent volume 50 mL) at temperature 20° C., with drying if possible, stirring for 60 s at 400 RPM.
17) Extract contents of separator with EtOAc (3×20 mL). Transfer waste phase (bottom) to waste, and product phase (top) directly to separator.
18) Wash contents of separator with NaCl (1×30 mL). Transfer waste phase (bottom) to waste, and product phase (top) directly to separator.
19) Transfer 150 mL from separator through MgSO₄ to rotavap at default speed, without flushing tubing after the transfer.
20) Transfer 20 mL from separator through MgSO₄ to rotavap at default speed, without flushing tubing after the transfer.
21) Evaporate contents of rotavap with pressure 100 mbar at temperature 40° C. for 60 min.

The crude product was purified by column chromatography (0 to 5% Et₂O/pet. ether).

The product allyl 1-methyl-2-oxocyclohexane-1-carboxylate SI-26 (1.31 g, 6.68 mmol, 74%) was obtained as a colourless oil.

¹H NMR (600 MHz, Chloroform-d) δ 5.89 (ddt, J=17.0, 10.7, 5.7 Hz, 1H), 5.31 (d, J=17.0 Hz, 1H), 5.24 (d, J=10.7 Hz, 1H), 4.76-4.58 (m, 2H), 2.61-2.39 (m, 3H), 2.10-1.96 (m, 1H), 1.79-1.61 (m, 3H), 1.51-1.40 (m, 1H), 1.31 (s, 3H). ¹³C NMR (151 MHz, Chloroform-d) δ 208.3, 173.0, 131.7, 119.0, 65.9, 57.4, 40.8, 38.4, 27.7, 22.8, 21.5.

### (S)-2-Allyl-2-methylcyclohexan-1-one 5

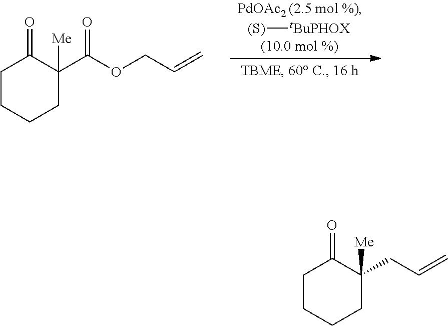

The synthesis was performed following the literature protocol by Stoltz et al. (9)

### Manual Preparations

The system was configured as specified in the graph file for this reaction. The corresponding reagent flask ‘SM’ was charged with a weighed amount of 1-methyl-2-oxo-cyclohexanecarboxylic acid 2-propenyl ester SI-26 (392 mg, 2.00 mmol, 1.0 eq.).

PdOAc₂ (11.2 mg, 0.05 mmol, 2.5 mol %) and (S)-tert-BuPHOX (77.4 mg, 0.20 mmol, 10 mol %) were added manually in the corresponding steps.

### ChemPU Steps

1) Evacuate reactor and refill with inert gas 3 times, using a vacuum pressure of 1 mbar, waiting 60 s after evacuating and 60 s after refilling with inert gas.
2) Add PdOAc₂ (11.2 mg, 0.05 mmol, 2.5 mol %) to reactor.
3) Add (S)-tert-ButylPHOX (77.4 mg, 0.20 mmol, 10 mol %) to reactor.
4) Evacuate reactor and refill with inert gas 1 times, using a vacuum pressure of 1 mbar, waiting 15 min after evacuating and 60 s after refilling with inert gas.
5) Add TBME (10 mL) directly to reactor at default speed with stirring at 250 RPM.
6) Asynchronous:
   a) Evacuate flask SM and refill with inert gas 1 times, using a vacuum pressure of 1 mbar, waiting 30 min after evacuating and 60 s after refilling with inert gas.
7) Heat/Chill reactor to 30° C. for 30 min with stirring at 250 RPM.
8) Repeat 2 times:
   a) Add TBME (3 mL) directly to SM at default speed without stirring.
   b) Transfer all from SM directly to reactor at default speed, flushing tubing after the transfer.
9) Heat/Chill reactor to 60° C. for 16 h with stirring at 250 RPM.
10) Allow to warm/cool reactor to 30° C. with stirring at 250 RPM. Temperature control is stopped once the temperature has been reached.
11) Transfer all from reactor through silica to rotavap at default speed, without flushing tubing after the transfer.
12) Repeat 3 times:
   a) Add diethylether (40 mL) directly to reactor at default speed with stirring at 250 RPM.
   b) Transfer all from reactor through silica to rotavap at default speed, without flushing tubing after the transfer.
13) Evaporate contents of rotavap with pressure 350 mbar at temperature 35° C. for 60 min.

The product (S)-2-allyl-2-methylcyclohexan-1-one 5 (279 mg, 1.84 mmol, 92%, 91.3:8.7 er) was obtained as a colourless oil. The recorded NMR signals are in agreement with the literature data. (8)

Enantiomeric excess determined by Chiral GC analysis using Chiraldex G-TA column (30 m×250 μm×0.12 μm). Assay conditions: He carrier gas (1.5 mL/min, 16.1 psi), 100° C. isothermal, 25 min. Retention times: 15.6 min (major), 18.5 min (minor). The absolute configuration of the product was previously reported in the literature. (8)

### 2-(3,4-dimethoxyphenyl)-1H-indole 6

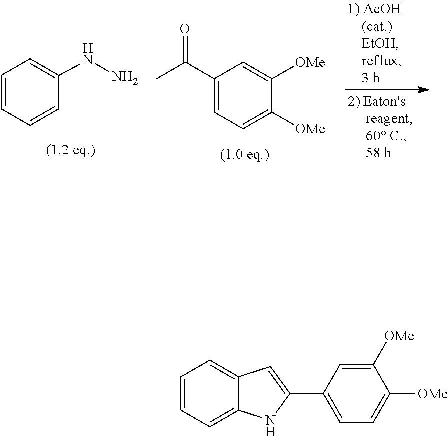

The synthesis was performed following a protocol adapted from the literature protocol by Reider et al. (10)

### Manual Preparations

The system was configured as specified in the graph file for this reaction. Acetoveratrone (2.70 g, 15 mmol, 1.0 eq.) was added manually in the corresponding step.

### ChemPU Steps

1) Add acetoveratrone (2.70 g, 15 mmol, 1.0 eq.) to reactor.
2) Add phenylhydrazine (1.8 mL, 18 mmol, 1.2 eq.) directly to reactor at default speed with stirring at 250 RPM.
3) Add ethanol (10 mL) directly to reactor at default speed with stirring at 250 RPM.
4) Add acetic acid (0.3 mL) directly to reactor at default speed with stirring at 250 RPM.
5) Add ethanol (20 mL) directly to reactor at default speed with stirring at 600 RPM.
6) Heat/Chill reactor to 70° C. for 3 h with stirring at 600 RPM.
7) Allow to warm/cool reactor to 55° C. with stirring at 250 RPM. Temperature control is stopped once the temperature has been reached.
8) Evaporate contents of reactor with pressure 20 mbar at temperature 60° C. for 2 h.
9) Allow to warm/cool reactor to 30° C. with stirring at 250 RPM. Temperature control is stopped once the temperature has been reached.
10) Add Eaton's reagent (15 mL) directly to reactor at default speed with stirring at 600 RPM.
11) Heat/Chill reactor to 60° C. for 48 h with stirring at 600 RPM.
12) Allow to warm/cool reactor to 30° C. with stirring at 250 RPM. Temperature control is stopped once the temperature has been reached.
13) Add Na₂CO₃ (20 mL) directly to reactor at default speed with stirring at 250 RPM.
14) Repeat 2 times:
   a) Add CH₂Cl₂ (20 mL) directly to reactor at default speed with stirring at 250 RPM.
   b) Add Na₂CO₃ (20 mL) directly to reactor at default speed with stirring at 250 RPM.
   c) Stop stirring reactor.
   d) Transfer all from reactor directly to separator at default speed, without flushing tubing after the transfer.
15) Extract contents of separator with CH₂Cl₂ (3×40 mL). Transfer waste phase (top) to waste, and product phase (bottom) directly to separator.
16) Transfer all from separator through MgSO₄ to rotavap at default speed, without flushing tubing after the transfer.
17) Transfer 30 mL from separator through MgSO₄ to rotavap at default speed, without flushing tubing after the transfer.
18) Evaporate contents of rotavap with pressure 50 mbar at temperature 40° C. for 60 min.

The crude product was purified by washing with methanol (3×10 mL). The product 2-(3,4-dimethoxyphenyl)-1H-indole 6 (2.35 g, 9.29 mmol, 62%) was obtained as a gray solid. The recorded NMR signals are in agreement with the literature data. (10)

### (3R,3aR,6R,7aR)-2-Benzyl-N-Cyclohexyl-1,2,3,6,7,7a-Hexahydro-3a,6-epoxyisoindole-3-carboxamide 7

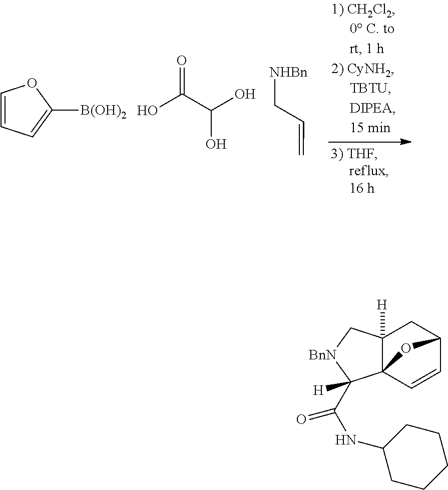

The synthesis was performed following literature protocol by Clausen et al. (11)

### Manual Preparation

The system was configured as specified in the graph file for this reaction. The filter reactor was charged with N-allylbenzylamine 7s3 (3.41 mL, 25.1 mmol, 1.00 equiv) and glyoxylic acid 7s2 (3.47 g, 37.7 mmol, 1.50 eq.) were loaded into the reactor flask. The solutions of 2-furanylboronic acid (1.25M), DIPEA (3.77M), cyclohexamine (3.0M) and TBTU (0.55M) in DCM were prepared manually.

### ChemPU Steps

1) Heat/Chill filter to 0° C. with stirring at 250 RPM. Temperature control is continued after the temperature has been reached.
2) Fill bottom of filter with CH₂Cl₂ (10 mL).
3) Add 2-furanylboronic acid in CH₂Cl₂ 7s1 (30.1 mL, 1.25 M, 4.22 g, 37.7 mmol, 1.5 eq.) directly to filter over 25 min with stirring at 250 RPM.
4) Stir filter for 10 min at 250 RPM stopping stirring afterwards.
5) Heat/Chill filter to 25° C. with stirring at 250 RPM. Temperature control is stopped once the temperature has been reached.
6) Stir filter for 50 min at 250 RPM stopping stirring afterwards.
7) Add CH₂Cl₂ (50 mL) directly to filter at default speed with stirring at 250 RPM.
8) Add H₂O (75 mL) directly to filter at default speed with stirring at 250 RPM.
9) Transfer all from filter directly to separator at default speed, flushing tubing after the transfer.
10) Extract two-phase mixture in separator without adding solvent. Transfer waste phase (top) to vessel separator, and product phase (bottom) directly to buffer flask.
11) Extract contents of separator with CH₂Cl₂ (2×50 mL). Transfer waste phase (top) to waste, and product phase (bottom) directly to separator.
12) Transfer all from buffer flask directly to separator at default speed, flushing tubing after the transfer.
13) Transfer 200 mL from separator directly to rotavap at default speed, flushing tubing after the transfer.
14) Evaporate contents of rotavap with pressure 450 mbar at temperature 40° C. for 20 min.
15) Dissolve contents of rotavap in CH₂Cl₂ (30 mL) at 25° C. over 20 min, stirring at 200 RPM.
16) Fill bottom of filter with CH₂Cl₂ (10 mL).
17) Transfer 100 mL from rotavap directly to filter at default speed, flushing tubing after the transfer.
18) Add DIPEA in CH₂Cl₂ (16.55 mL, 3.77 M, 37.7 mmol, 1.5 eq.) directly to filter at default speed with stirring at 250 RPM.
19) Add TBTU in CH₂Cl₂ (50 mL, 0.55M, 27.6 mmol, 1.1 eq.) directly to filter at default speed with stirring at 250 RPM.
20) Heat/Chill filter to 0° C. with stirring at 250 RPM. Temperature control is continued after the temperature has been reached.
21) Add cyclohexylamine in CH₂Cl₂ (10 mL, 3.0 M, 30.1 mmol, 1.2 eq.) directly to filter over 20 min with stirring at 250 RPM.
22) Stir filter for 10 min at 250 RPM leaving stirring on afterwards.
23) Heat/Chill filter to 25° C. with stirring at 250 RPM. Temperature control is stopped once the temperature has been reached.
24) Stir filter for 10 min at 250 RPM leaving stirring on afterwards.
25) Add aqueous NaHCO₃ (50 mL) directly to filter at default speed with stirring at 250 RPM.
26) Transfer all from filter directly to separator at default speed, flushing tubing after the transfer.
27) Extract two-phase mixture in separator without adding solvent. Transfer waste phase (top) to vessel separator, and product phase (bottom) directly to a buffer flask.
28) Extract contents of separator with CH₂Cl₂ (2×50 mL). Transfer waste phase (top) to waste, and product phase (bottom) directly to separator.
29) Transfer all from buffer flask directly to separator at default speed, flushing tubing after the transfer.
30) Transfer 200 mL from separator directly to rotavap at default speed, flushing tubing after the transfer.
31) Evaporate contents of rotavap with default pressure control at temperature 40° C. for 30 min.
32) Shut down the platform.

The product, (3R,3aR,6R,7aR)-2-Benzyl-N-cyclohexyl-1,2,3,6,7,7a-hexahydro-3a,6-epoxyisoindole-3-carboxamide (5.3 g, 15 mmol, 60%) was obtained as a red-brown solid. The recorded NMR signals are in agreement with the literature data. (11)

### Ethyl (6a,11a)-6-benzyl-9-methoxy-5-oxo-5,6,6a,11-tetrahydro-11aH-indeno[1,2-c]isoquinoline-11a-carboxylate 8

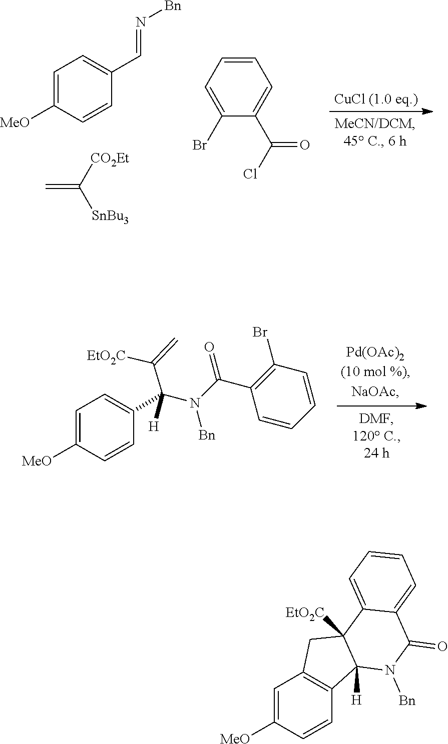

The synthesis was performed following the literature protocol by Malinakova et al. (12, 13)

### Manual Preparations

A solution of imine 8s1 (575 mg) in dry acetonitrile (total volume 10 mL, 0.255 M), a solution of acyl chloride 8s2 (728 mL) in dry acetonitrile (total volume 10 mL, 0.332 M) and a solution of stannane 8s3 (384 mg) in dry dichloromethane (total volume 5 mL, 0.197 M) were prepared. The reactor 1 was charged with CuCl (50.0 mg, 0.500 mmol, 1.0 eq.) and 3 Å molecular sieves (300 mg). Silica (3.8 g) and KF (0.94 g) were grinded and mixed in a mortar and packed in a 4 g cartridge. Pd(OAc)₂ (11.3 mg, 0.05 mmol, 0.1 eq.) and NaOAc (41.4 mg, 0.50 mmol, 1.0 eq.) were added manually in the corresponding steps.

### ChemPU Steps

1) Dry contents of reactor 2 for 60 min at pressure 10 mbar at temperature 120°. The heating is stopped when the step finishes.
2) Dry contents of reactor 1 for 60 min at pressure 10 mbar at temperature 120°. The heating is stopped when the step finishes.
3) Evacuate reactor 1 and refill with inert gas 3 times, using a vacuum pressure of 10 mbar, waiting 60 s after evacuating and 60 s after refilling with inert gas.
4) Heat/Chill reactor 1 to 25° C. with stirring at 250 rpm. The temperature control is continued after the temperature has been reached.
5) Add imine 8s1 acetonitrile solution (1.96 mL, 0.255 M, 0.500 mmol, 1.0 eq.) directly to reactor 1 at default speed with stirring at 250 rpm.
6) Add aryl chloride 8s2 acetonitrile solution (1.96 mL, 0.332 M, 0.650 mmol, 1.3 eq.) directly to reactor 1 at default speed with stirring at 250 rpm.
7) Heat/Chill reactor 1 to 25° C. for 5 min with stirring at 250 rpm.
8) Add stannane 8s3 DCM solution (4 mL, 0.197 M, 0.750 mmol, 1.5 eq.) directly to reactor 1 at default speed without stirring.
9) Heat/Chill reactor 1 to 45° C. for 6 h with stirring at 250 rpm.
10) Heat/Chill reactor 1 to 25° C. with stirring at 250 rpm. Temperature control is continued after the temperature has been reached.
11) Transfer 12 mL from reactor 1 through KF-silica to reactor 2 at default speed, without flushing tubing after the transfer.
12) Repeat 3 times:
   a) Add dichloromethane (10 mL) directly to reactor 1 at default speed without stirring.
   b) Stir reactor 1 for 60 s at 600 rpm stopping stirring afterwards.
   c) Transfer 15 mL from reactor 1 through KF-silica to reactor 2 at default speed, without flushing tubing after the transfer.
13) Transfer 50 mL from reactor 1 through KF-silica to reactor 2 at default speed, without flushing tubing after the transfer.
14) Evaporate contents of reactor 2 with default pressure control at temperature 50° C. for 5 h.
15) Dry contents of reactor 2 for 60 min at pressure 3 mbar at temperature 70° C. The heating is stopped when the step finishes.
16) Add NaOAc (41.4 mg, 0.50 mmol, 1.0 eq.) to reactor 2.
17) Add Pd(OAc)₂ (11.3 mg, 0.05 mmol, 0.1 eq.) to reactor 2.
18) Evacuate reactor 2 and refill with inert gas 5 times, using a vacuum pressure of 50 mbar, waiting 60 s after evacuating and 60 s after refilling with inert gas.
19) Add anhydrouse DMF (2 mL) directly to reactor 2 at default speed without stirring.
20) Heat/Chill reactor 2 to 120° C. for 24 h with stirring at 250 rpm.
21) Heat/Chill reactor 2 to 25° C. with stirring at 250 rpm. Temperature control is continued after the temperature has been reached.
22) Add ethyl acetate (100 mL) directly to reactor 2 at default speed without stirring.
23) Transfer 120 mL from reactor 2 directly to separator at default speed, without flushing tubing after the transfer.
24) Repeat 2 times:
   a) Add ethyl acetate (25 mL) directly to reactor 2 at default speed without stirring.
   b) Stir reactor 2 for 60 s at 600 rpm stopping stirring afterwards.
   c) Transfer 40 mL from reactor 2 directly to separator at default speed, without flushing tubing after the transfer.
25) Repeat 2 times:
   a) Add water (20 mL) directly to separator at default speed without stirring.
   b) Wash contents of separator with brine (1×100 mL). Transfer waste phase (bottom) to waste, and product phase (top) directly to separator.
26) Add water (20 mL) directly to separator at default speed without stirring.
27) Wash contents of separator with brine (1×100 mL). Transfer waste phase (bottom) to waste, and product phase (top) directly to separator.
28) Transfer 200 mL from separator through MgSO₄ to rotavap at default speed, without flushing tubing after the transfer.
29) Transfer 30 mL from EtOAc through MgSO₄ to rotavap at default speed, without flushing tubing after the transfer.
30) Transfer 50 mL from argon source through MgSO₄ to rotavap at default speed, without flushing tubing after the transfer.
31) Evaporate contents of rotavap with default pressure control at temperature 25° C. for 30 min.
32) Dry contents of rotavap for 30 min at pressure 10 mbar at temperature 50° C. stopping heating when step finishes.
33) Shut down the platform.

The crude product was purified by flash column chromatography (12 g Buchi ‘EcoFlex’ silica column, 40-63 μm particle size, 55-75 Å pore size, irregular particle shape) using a gradient of ethyl acetate in petroleum ether (0%-25% over 20 column volumes).

The product ethyl (6a,11a)-6-benzyl-9-methoxy-5-oxo-5,6,6a,11-tetrahydro-11aH-indeno[1,2-c]isoquinoline-11a-carboxylate 8 was obtained as a white solid (133 mg, 0.228 mmol, 46%). The recorded NMR signals are in agreement with the literature data. (13)

### Library Synthesis of 9a, 9b, 9c and 9d

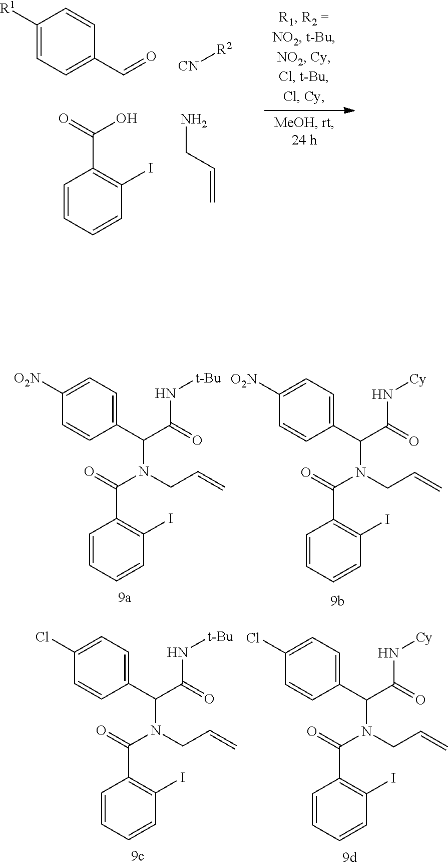

The syntheses were performed following the literature protocol by Yang et al. (14). All four syntheses were run in parallel.

### Manual Preparations

The system was configured as specified in the graph file for this reaction.

tert-Butyl-isocyanide (997 mg, 12.0 mmol) was added to ‘reactor3’ and ‘reactor5’ and cyclohexyl isocyanide (1.31 g, 12.0 mmol) was added to ‘reactor4’ and ‘reactor6’ prior to the start of the automated run. 4-nitrobenzaldehyde (3.62 g, 24.0 mmol) and 4-chlorobenzaldehyde (3.89 g, 24.0 mmol) were added manually in the corresponding steps.

### ChemPU Steps

1) Add 4-nitrobenzaldehyde (3.62 g, 24.0 mmol) to reactor 1.
2) Add 4-chlorobenzaldehyde (3.89 g, 24.0 mmol) to reactor 2.
3) Add 1 M allylamine methanol solution (24.0 mL, 24.0 mmol) to reactor 1.
4) Add 1 M allylamine methanol solution (24.0 mL, 24.0 mmol) to reactor 2.
5) Start stirring reactors 1 and 2.
6) Wait 10 minutes.
7) Add 1 M 2-iodobenzoic acid solution (24 mL, 24.0 mmol) to reactor 1.
8) Add 1 M 2-iodobenzoic acid solution (24 mL, 24.0 mmol) to reactor 2.
9) Wait 10 minutes.
10) Add t-butyl isocyanide (997 mg, 12.0 mmol) to reactor 3.
11) Add cyclohexyl isocyanide (1.31 g, 12.0 mmol) to reactor 4.
12) Add t-butyl isocyanide (997 mg, 12.0 mmol) to reactor 5.
13) Add cyclohexyl isocyanide (1.31 g, 12.0 mmol) to reactor 6.
14) Transfer 24 mL of the reaction mixture in reactor 1 to reactor 3.
15) Transfer 24 mL of the reaction mixture in reactor 1 to reactor 4.
16) Transfer 24 mL of the reaction mixture in reactor 2 to reactor 5.
17) Transfer 24 mL of the reaction mixture in reactor 2 to reactor 6.
18) Wait 24 hours.
19) Filter contents of reactor 3, sending the liquid phase to waste.
20) Filter contents of reactor 4, sending the liquid phase to waste.
21) Filter contents of reactor 5, sending the liquid phase to waste.
22) Filter contents of reactor 6, sending the liquid phase to waste.

The products N-allyl-N-(2-(tert-butylamino)-1-(4-nitrophenyl)-2-oxoethyl)-2-iodobenzamide 9a (4.16 g, 7.98 mmol, 67%), N-allyl-N-(2-(cyclohexylamino)-1-(4-nitrophenyl)-2-oxoethyl)-2-iodobenzamide 9b (5.44 g, 9.94 mmol, 83%), N-allyl-N-(2-(tert-butylamino)-1-(4-chlorophenyl)-2-oxoethyl)-2-iodobenzamide 9c (4.16 g, 8.14 mmol, 68%), N-allyl-N-(1-(4-chlorophenyl)-2-(cyclohexylamino)-2-oxoethyl)-2-iodobenzamide 9d (5.22 g, 9.72 mmol, 81%) were obtained as white to tan powders. HPLC/DAD analysis shows a single peak for each of 9a, 9b, and 9d, with 9c showing a small impurity peak.

The recorded NMR signals are in agreement with the literature data. (14)

### Dimethyl 2-(3-methylbut-2-en-1-yl)malonate 10

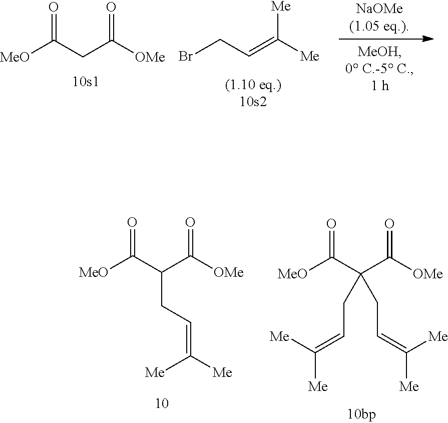 [SMILES: COC(=O)CC(=O)OC ; CC(C)=CCBr ; COC(=O)C(CC=C(C)C)C(=O)OC ; COC(=O)C(CC=C(C)C)(CC=C(C)C)C(=O)OC]

The synthesis was performed following the literature protocol by Mook and Sher. (15)

### Manual Preparations

NaOMe (1.16 g, 21.0 mmol, 1.05 eq.) was dissolved in methanol (10 mL). The ‘Prenyl bromide’ reagent vial was charged with a weighed amount of prenyl bromide 10s2 (3.67 g, 22.1 mmol, 1.10 eq.). Prenyl bromide was stored over a small amount of silver powder and was shielded from light. A mixture of brine and saturated aqueous NaHCO₃ (1:1 v/v) was prepared.

### ChemPU Steps

1) Add sodium methoxide (1.16 g, 21.0 mmol, 1.05 eq.) in methanol (10 mL) directly to reactor at default speed without stirring.
2) Repeat 2 times:
   a) Add methanol (5 mL) directly to sodium methoxide vial at default speed without stirring.
   b) Add argon (20 mL) directly to sodium methoxide vial at default speed without stirring.
   c) Add all from sodium methoxide vial directly to reactor at default speed without stirring.
3) Add dimethyl malonate 10s1 (2.32 mL, 20.0 mmol, 1.00 eq.) directly to reactor at default speed with stirring at 250 RPM.
4) Heat/Chill reactor to 25° C. for 60 min with stirring at 250 RPM.
5) Heat/Chill reactor to 3° C. with stirring at 250 RPM. Temperature control is continued after the temperature has been reached.
6) Add 10 mL from prenyl bromide vial directly to reactor over 20 min with stirring at
250 RPM.
7) Repeat 2 times:
   a) Add methanol (3 mL) directly to prenyl bromide vial at default speed without stirring.
   b) Add argon (10 mL) directly to prenyl bromide vial at default speed without stirring.
   c) Add all from prenyl bromide vial directly to reactor at default speed with stirring at 250 RPM.
8) Heat/Chill reactor to 5° C. for 4 h with stirring at 250 RPM.
9) Evaporate contents of reactor with pressure 50 mbar at temperature 50° C. for 2 h.
10) Repeat 3 times:
   a) Add diethyl ether (22 mL) directly to reactor at default speed with stirring at 250 RPM.
   b) Stir reactor for 5 min at 250 RPM stopping stirring afterwards.
   c) Transfer all from reactor directly to separator at default speed, without flushing tubing after the transfer.
11) Add water (5 mL) directly to separator at default speed without stirring.
12) Wash contents of separator with aq NaHCO₃-brine (1×40 mL). Transfer waste phase (bottom) to vessel separator, and product phase (top) through MgSO₄ to rotavap.
13) Extract contents of separator with diethyl ether (3×30 mL). Transfer waste phase (bottom) to waste, and product phase (top) through MgSO₄ to rotavap.
14) Transfer 20 mL from Et₂O through MgSO₄ to rotavap at default speed, without flushing tubing after the transfer.
15) Transfer 50 mL from flask_argon_bb through MgSO₄ to rotavap at default speed, without flushing tubing after the transfer.
16) Evaporate contents of rotavap with pressure 218 mbar at temperature 50° C. for 30 min.
17) Dry contents of rotavap for 10 min at default pressure at temperature 50° C. stopping heating when step finishes.

The crude product was purified by flash column chromatography (100 g Buchi ‘EcoFlex’ silica column, 40-63 μm particle size, 55-75 A pore size, irregular particle shape) using a gradient of ethyl acetate in petroleum ether (0%-100% over 14 column volumes).

The product dimethyl 2-(3-methylbut-2-en-1-yl)malonate 10 was obtained as a colourless oil (2.47 g, 12.3 mmol, 56%). The recorded NMR signals are in agreement with the literature data. (17)

The experiment above was re-run 12 times. The yield and purity of the crude material was determined by ¹H-NMR taking into account the ratio of double-alkylated product 10 bp.

| | | | | By- | | | |
|---|---|---|---|---|---|---|---|
| | | Mass | Product | product | | | Total |
| | | crude | 10 | 10 bp | Purity | Yield | runtime |
| Exp. No. | Pass/Fail | (g) | signal | signal | (%) | (%) | (min) |
| 1 | Pass | 3.8747 | 1 | 0.03 | 98 | 94.8 | n/a |
| 2 | Pass | 3.7516 | 1 | 0.08 | 95 | 88.9 | 1629.3 |
| 3 | Fail | — | — | — | — | — | n/a |
| 4 | Pass | 3.9771 | 1 | 0.07 | 96 | 94.9 | 1629.7 |
| 5 | Pass | 4.0062 | 1 | 0.08 | 95 | 94.9 | 1625.4 |
| 6 | Pass | 3.9949 | 1 | 0.07 | 96 | 95.3 | 1623.8 |
| 7 | Pass | 3.9120 | 1 | 0.07 | 96 | 93.3 | 1648.3 |
| 8 | Pass | 3.9760 | 1 | 0.06 | 96 | 95.4 | 1626.2 |
| 9 | Pass | 3.883 | 1 | 0.09 | 94 | 91.4 | 1649.8 |
| 10 | Pass | 3.974 | 1 | 0.08 | 95 | 94.2 | n/a |
| 11 | Fail | — | — | — | — | — | n/a |
| 12 | Pass | 3.9268 | 1 | 0.07 | 96 | 93.7 | 1636.4 |
| | Min | 3.7516 | | | 94.3 | 88.9 | 1623.8 |
| | Max | 4.0062 | | | 98.0 | 95.4 | 1649.8 |
| | Average | 3.9276 | | | 95.5 | 93.7 | 1633.6 |
| | Std | 0.0772 | | | 1.0 | 2.1 | 10.265 |
| | % Error | 1.97 | | | 1.06 | 2.20 | 0.63 |

### Tropinone 11

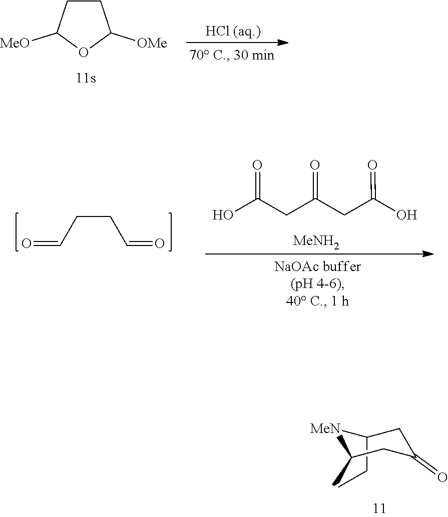

The synthesis was performed following the literature protocol by Williams et a). (18)

### Manual Preparations

The system was configured as specified in the graph file for this reaction. Acetone dicarboxylic acid (3.21 g, 22.0 mmol, 1.1 eq.), sodium acetate (6.56 g, 80.0 mmol, 4.0 eq.), and methylamine hydrochloride (1.49 g, 22.0 mmol, 1.1 eq.) were added manually in the corresponding steps.

### ChemPU Steps

1) Add 2,5-dimethoxytetrahydrofuran (2.59 mL, 20.0 mmol, 1.0 eq.) directly to intermediate at default speed without stirring.
2) Add 0.5M HCl (6.5 mL) directly to intermediate at default speed with stirring at 250 RPM.
3) Heat/Chill intermediate to 80° C. for 40 min with stirring at 250 RPM.
4) Heat/Chill intermediate to 10° C. with stirring at 250 RPM. Temperature control is continued after the temperature has been reached.
5) Add acetone dicarboxylic acid (3.21 g, 22.0 mmol, 1.1 eq.) to reactor. 6) Add sodium acetate (6.56 g, 80.0 mmol, 4.0 eq.) to reactor.
7) Add methylamine hydrochloride (1.49 g, 22.0 mmol, 1.1 eq.) to reactor.
8) Add water (25 mL) directly to reactor at default speed with stirring at 250 RPM.
9) Heat/Chill reactor to 5° C. for 15 min with stirring at 250 RPM.
10) Transfer 15 mL from intermediate directly to reactor over 10 min, flushing tubing after the transfer.
11) Add water (5 mL) directly to intermediate at default speed with stirring at 250 RPM.
12) Transfer 10 mL from intermediate directly to reactor at default speed, flushing tubing after the transfer.
13) Heat/Chill reactor to 45° C. for 60 min with stirring at 250 RPM.
14) Heat/Chill reactor to 20° C. with stirring at 250 RPM. Temperature control is stopped once the temperature has been reached.
15) Add 50% NaOH (3 mL) directly to reactor at default speed with stirring at 250 RPM.
16) Add NaCl (15 mL) directly to reactor at default speed with stirring at 250 RPM.
17) Stir reactor for 10 min at 250 RPM stopping stirring afterwards.
18) Repeat 2 times:
   a) Add water (10 mL) directly to reactor at default speed with stirring at 250 RPM.
   b) Add CH₂Cl₂ (20 mL) directly to reactor at default speed with stirring at 250 RPM.
   c) Stop stirring reactor.
   d) Transfer all from reactor directly to separator at default speed, without flushing tubing after the transfer.
19) Extract contents of separator with CH₂Cl₂ (3×30 mL). Transfer waste phase (top) to waste, and product phase (bottom) directly to separator.
20) Transfer all from separator through MgSO₄ to rotavap at default speed, without flushing tubing after the transfer.
21) Evaporate contents of rotavap with pressure 20 mbar at temperature 40° C. for 60 min.

The product tropinone 11 (2.26 g, 16.3 mmol, 81%) was obtained as a pale yellow solid. The recorded NMR signals are in agreement with the literature data. (18)

### Tropine 12

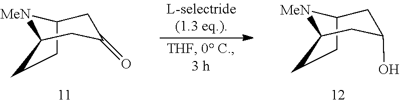 [SMILES: CN1C2CC[C@@H]1CC(=O)C2 ; CN1C2CC[C@@H]1CC(O)C2]

The synthesis was performed following a protocol adapted from protocol reported by Pierre-Antoine Nocquet and Till Opatz. (19)

### Manual Preparations

The system was configured as specified in the graph file for this reaction.

Tropinone (1.39 g, 10.0 mmol, 1.0 eq.) was added manually in the corresponding step.

### ChemPU Steps

1) Add tropinone (1.39 g, 10.0 mmol, 1.0 eq.) to reactor.
2) Add tetrahydrofuran (70 mL) directly to reactor at default speed with stirring at 250 RPM.
3) Heat/Chill reactor to 0° C. with stirring at 250 RPM. Temperature control is continued after the temperature has been reached.
4) Add L-selectride (13.0 mL, 1.0 M in THF, 13.0 mmol, 1.3 eq.) directly to reactor over 10 min with stirring at 250 RPM.
5) Heat/Chill reactor to 0° C. for 3 h with stirring at 250 RPM.
6) Add conc. NaOH (20 mL) directly to reactor over 10 min with stirring at 250 RPM.
7) Add 30% H₂O₂ (6 mL) directly to reactor over 5 min with stirring at 250 RPM.
8) Heat/Chill reactor to 20° C. with stirring at 250 RPM. Temperature control is stopped once the temperature has been reached.
9) Stir reactor for 60 min at 250 RPM stopping stirring afterwards.
10) Transfer all from reactor directly to separator at default speed, without flushing tubing after the transfer.
11) Repeat 2 times:
   a) Add water (20 mL) directly to reactor at default speed with stirring at 250 RPM.
   b) Add CHCl₃ (20 mL) directly to reactor at default speed with stirring at 250 RPM.
   c) Stop stirring reactor.
   d) Transfer all from reactor directly to separator at default speed, without flushing tubing after the transfer.
12) Add NaHCO₃ (30 mL) directly to separator at default speed without stirring.
13) Extract contents of separator with CHCl₃ (3×40 mL). Transfer waste phase (top) to waste, and product phase (bottom) directly to separator.
14) Transfer all from separator through MgSO₄ to rotavap at default speed, flushing tubing after the transfer.
15) Evaporate contents of rotavap with pressure 50 mbar at temperature 40° C. for 60 min.

The crude product (containing iso-butanol) was purified by converting it to the hydrochloride salt. The crude oily solid was dissolved in Et₂O and filtered through a plug of cotton. To the filtrate was added HCl (2M in Et₂O, 6.0 mL, 12.0 mmol), resulting in an immediate precipitation of the salt. The resulting solid was filtered, washed with Et₂O (3×20 mL), and dried in vacuo.

The product tropine hydrochloride 12-HCl (1.49 g, 8.39 mmol, 84%) was obtained as a white solid. The recorded NMR signals are in agreement with the literature data. (20)

### Atropine 13

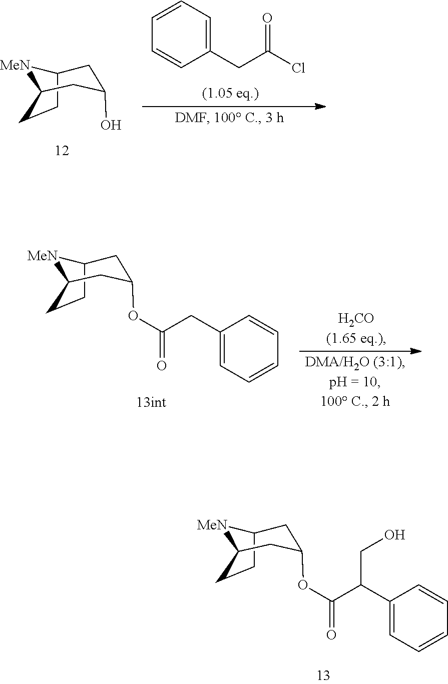 [SMILES: CN1C2CC[C@@H]1CC(O)C2 ; O=C(Cl)Cc1ccccc1 ; CN1C2CC[C@@H]1CC(OC(=O)Cc1ccccc1)C2 ; CN1C2CC[C@@H]1CC(OC(=O)C(CO)c1ccccc1)C2]

The synthesis was performed following the literature protocol by Jamison et al. (20)

### Manual Preparations

The system was configured as specified in the graph file for this reaction.

Tropine (2.82 g, 20.0 mmol, 1.0 eq.) was added manually in the corresponding step.

### ChemPU Steps

1) Add tropine (2.82 g, 20.0 mmol, 1.0 eq.) to reactor.
2) Add 2M HCl in Et₂O (10.0 mL, 20.0 mmol) directly to reactor at default speed with stirring at 250 RPM.
3) Add DMF (50 mL) directly to reactor at default speed with stirring at 250 RPM.
4) Stir reactor for 5 min at 400 RPM stopping stirring afterwards.
5) Add phenylacetylchloride (2.80 mL, 21.0 mmol, 1.05 eq.) directly to reactor at default speed with stirring at 250 RPM.
6) Add DMF (10 mL) directly to reactor at default speed with stirring at 250 RPM.
7) Heat/Chill reactor to 100° C. for 3 h with stirring at 250 RPM.
8) Allow to warm/cool reactor to 30° C. with stirring at 250 RPM. Temperature control is stopped once the temperature has been reached.
9) Transfer all from reactor directly to separator at default speed, without flushing tubing after the transfer.
10) Repeat 2 times:
   a) Add CH₂Cl₂ (30 mL) directly to reactor at default speed with stirring at 250 RPM.
   b) Stop stirring reactor.
   c) Transfer all from reactor directly to separator at default speed, without flushing tubing after the transfer.
11) Add NaHCO₃ (60 mL) directly to separator at default speed without stirring.
12) Extract contents of separator with CH₂Cl₂ (3×40 mL). Transfer waste phase (top) to waste, and product phase (bottom) directly to separator.
13) Wash contents of separator with water (4×50 mL). Transfer waste phase (top) to waste, and product phase (bottom) directly to separator.
14) Wash contents of separator with NaCl (2×50 mL). Transfer waste phase (top) to waste, and product phase (bottom) directly to separator.
15) Transfer all from separator through MgSO₄ to rotavap at default speed, flushing tubing after the transfer.
16) Evaporate contents of rotavap with pressure 20 mbar at temperature 40° C. for 60 min.

The product a-phenyl acetyl tropine 13 int (3.49 g, 13.5 mmol, 67%) was obtained as a yellow oil containing DMF and was used without purification. The recorded NMR signals are in agreement with the literature data. (20) Manual preparations

The system was configured as specified in the graph file for this reaction. The corresponding reagent flask ‘phenylacetyltropine’ was charged with a weighed amount of a-phenyl acetyl tropine (3.37 g, 13.0 mmol, 1.00 eq.). A pH 10 buffer was prepared by mixing 1.0 M Na₂CO₃ and 1.0 M NaHCO₃ solutions in 1:1 ratio.

### ChemPU Steps

1) Repeat 3 times:
   a) Add DMA (5 mL) directly to phenylacetyltropine at default speed without stirring.
   b) Transfer all from phenylacetyltropine directly to reactor at default speed, without flushing tubing after the transfer.
2) Add DMA (25 mL) directly to reactor at default speed with stirring at 250 RPM.
3) Add basic buffer (5 mL) directly to reactor at default speed with stirring at 250 RPM.
4) Add 37% formaldehyde (1.6 mL, 21.5 mmol, 1.65 eq) directly to reactor at default speed with stirring at 250 RPM.
5) Add basic buffer (10 mL) directly to reactor at default speed with stirring at 250 RPM.
6) Heat/Chill reactor to 100° C. for 2 h with stirring at 250 RPM.
7) Allow to warm/cool reactor to 30° C. with stirring at 250 RPM. Temperature control is stopped once the temperature has been reached.
8) Repeat 2 times:
   a) Add CH₂Cl₂ (30 mL) directly to reactor at default speed with stirring at 250 RPM.
   b) Add water (30 mL) directly to reactor at default speed with stirring at 250 RPM.
   c) Stop stirring reactor.
   d) Transfer all from reactor directly to separator at default speed, without flushing tubing after the transfer.
9) Add NaHCO₃ (60 mL) directly to separator at default speed without stirring.
10) Extract contents of separator with CH₂Cl₂ (2×40 mL). Transfer waste phase (top) to waste, and product phase (bottom) directly to separator.
11) Wash contents of separator with water (5×50 mL). Transfer waste phase (top) to waste, and product phase (bottom) directly to separator.
12) Wash contents of separator with NaCl (2×50 mL). Transfer waste phase (top) to waste, and product phase (bottom) directly to separator.
13) Transfer all from separator through MgSO4 to rotavap at default speed, without flushing tubing after the transfer.
14) Evaporate contents of rotavap with pressure 20 mbar at temperature 40° C. for 60 min.
15) Evaporate contents of rotavap with pressure 10 mbar at temperature 40° C. for 30 min.

The crude product was purified by column chromatography (5% MeOH/1% Et₃N/CH₂Cl₂).

The product atropine 13 (1.37 g, 4.75 mmol, 37%, 25% over two steps from 12) was obtained as an off-white solid. The recorded NMR signals are in agreement with the literature data. (20)

### N-(pyridin-2-ylmethyl)thiophen-2-amine 14

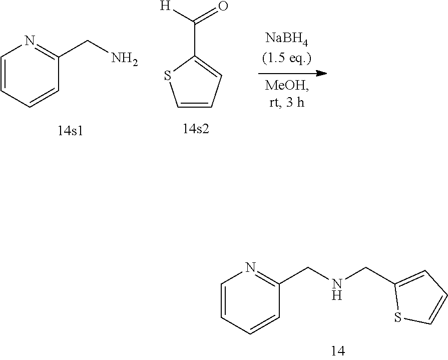 [SMILES: NCc1ccccn1 ; O=Cc1cccs1 ; c1ccc(CNCc2cccs2)nc1]

The synthesis was performed according to the modified literature protocol by Zubieta et al. (21) Manual preparation

The reactor_2 was charged with NaBH₄ (1.16 g, 30.0 mmol, 1.50 eq.).

### ChemPU Steps

1) Add methanol (6 mL) directly to reactor_1 at default speed with stirring at 250 RPM.
2) Add 2-thiophenecarboxaldehyde 14s2 (1.91 mL, 20.0 mmol, 1.0 eq.) directly to reactor_1 at default speed with stirring at 250 RPM.
3) Add 2-picolylamine 14s1 (2.08 mL, 20.0 mmol, 1.0 eq.) directly to reactor_1 at default speed with stirring at 250 RPM.
4) Heat/Chill reactor_1 to 25° C. for 60 min with stirring at 250 RPM.
5) Stir reactor_1 for 60 s at 250 RPM leaving stirring on afterwards. The reactors should be stirred during the transfer in the next step.
6) Transfer all from reactor_1 directly to reactor_2 at default speed, without flushing tubing after the transfer.
7) Heat/Chill reactor_2 to 25° C. for 3 h with stirring at 250 RPM.
8) Evaporate contents of reactor_2 with default pressure control at temperature 45° C. for 2 h.
9) Dissolve contents of reactor_2 in dichloromethane (50 mL) at 25° C. over 5 min, stirring at 250 RPM.
10) Transfer all from reactor_2 directly to separator at default speed, without flushing tubing after the transfer.
11) Dissolve contents of reactor_2 in water (50 mL) at 25° C. over 5 min, stirring at 250 RPM.
12) Transfer all from reactor_2 directly to separator at default speed, without flushing tubing after the transfer.
13) Dissolve contents of reactor_2 in dichloromethane (50 mL) at 25° C. over 5 min, stirring at 250 RPM.
14) Transfer all from reactor_2 directly to separator at default speed, without flushing tubing after the transfer.
15) Separate mixture in separator, sending lower phase through MgSO₄ to rotavap and upper phase directly to separator.
16) Extract contents of separator with dichloromethane (2×50 mL). Transfer waste phase (top) to waste, and product phase (bottom) through MgSO₄ to rotavap.
17) Evaporate contents of rotavap with default pressure control at temperature 50° C. for 30 min.
18) Dry contents of rotavap for 30 min at pressure 50 mbar at temperature 50° C. stopping heating when step finishes.
19) Add dichloromethane (1 mL) directly to rotavap at default speed without stirring.
20) Add petroleum ether (1 mL) directly to rotavap at default speed without stirring.
21) Run preparative chromatography with DCM in petroleum ether (50% to 100% over 5 column volumes) followed by methanol in DCM (0% to 15% over 10 column volumes).
22) Evaporate contents of rotavap with default pressure control at temperature 45° C. for 30 min.
23) Evaporate contents of rotavap with default pressure control at temperature 45° C. for 30 min.
24) Dry contents of rotavap for 30 min at default pressure at temperature 45° C. stopping heating when step finishes.

The product N-(pyridin-2-ylmethyl)thiophen-2-amine 14 was obtained as a pale yellow oil (2.27 g, 11.1 mmol, 62%). The recorded NMR signals in methanol-d₄ are in agreement with the literature data. (21)

### (R)-4-Phenyl-3-(2-triisopropylsilyl-ethynyl)oxazolidin-2-one 15

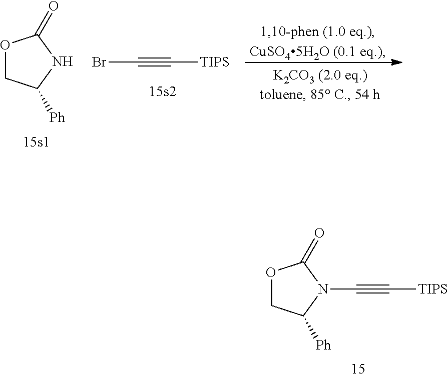

The synthesis was performed following the literature protocol by Sagamanova et al. (22)

### Manual Preparations

2-Bromoethynyl-tri(propan-2-yl)silane 15s2 (3.48 g, 12.0 mmol, 1.2 eq.) was charged in the flask labelled ‘alkyne_vial’. CuSO₄·5H₂O and K₂CO₃ were finely ground in a mortar. The reactor was charged with 1,10-phenantroline (1.65 g, 10.0 mmol, 1.0 eq.), R-phenyloxazolidinone 15s1 (1.65 g, 10.0 mmol, 1.0 eq.), CuSO₄·5H₂0 (252 mg, 1.00 mmol, 0.1 eq.), and K₂CO₃ (2.79 g, 20.0 mmol, 2.0 eq.).

### ChemPU Steps

1) Evacuate reactor and refill with inert gas 3 times, using a vacuum pressure of 50 mbar, waiting 60 s after evacuating and 60 s after refilling with inert gas.
2) Add toluene (7.27 mL) directly to alkyne_vial at default speed with stirring at 250 RPM.
3) Transfer 400 mL from flask_argon_2 directly to alkyne_vial at default speed, without flushing tubing after the transfer.
4) Transfer 10 mL from alkyne_vial directly to reactor at default speed, without flushing tubing after the transfer.
5) Heat/Chill reactor to 85° C. for 54 h with stirring at 250 RPM.
6) Repeat 5 times:
   a) Add ethyl acetate (13 mL) directly to reactor at default speed with stirring at 250 RPM.
   b) Add petroleum ether (13 mL) directly to reactor at default speed with stirring at 250 RPM.
   c) Transfer all from reactor through Celite_and_silica to rotavap at default speed, without flushing tubing after the transfer.
7) Transfer 200 mL from flask_argon_2 through Celite_and_silica to rotavap at default speed, without flushing tubing after the transfer.
8) Evaporate contents of rotavap with pressure 218 mbar at temperature 45° C. for 30 min.
9) Evaporate contents of rotavap with pressure 153 mbar at temperature 45° C. for 30 min.
10) Evaporate contents of rotavap with pressure 48 mbar at temperature 45° C. for 30 min.
11) Dry contents of rotavap for 60 min at pressure 2 mbar at temperature 70° C. stopping heating when step finishes.
12) Dissolve contents of rotavap in petroleum ether (7.5 mL) at 30° C. over 5 min, stirring at 400 RPM.
13) Run preparative chromatography with ethyl acetate in petroleum ether (0% to 20% over 8 column volumes).
14) Evaporate contents of rotavap with default pressure control at temperature 45° C. for 30 min.
15) Evaporate contents of rotavap with default pressure control at temperature 45° C. for 30 min.
16) Dry contents of rotavap for 30 min at default pressure at temperature 45° C. stopping heating when step finishes

The product (R)-4-phenyl-3-(2-triisopropylsilyl-ethynyl)oxazolidin-2-one 15 (2.48 g, 7.22 mmol, 72%) was obtained as a pale yellow oil. The recorded NMR signals are in agreement with the literature data. (22)

### Benzyl 4-nitrobenzoate SI-1

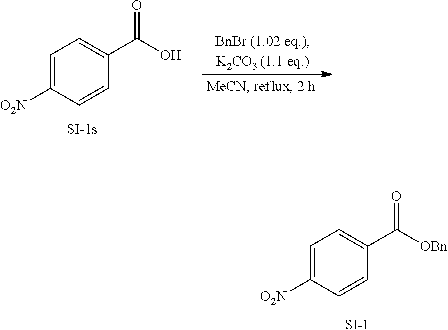

The synthesis was performed following the literature protocol by Tokuyama et al. (23)

### Manual Preparation

The system was configured as specified in the graph file for this reaction. The reactor was charged with 5-nitrobenzoic acid (0.83 g, 5.00 mmol, 1.00 eq.) and potassium carbonate (0.76 g, 5.55 mmol, 1.11 eq.).

### ChemPU Steps

1) Add MeCN (50 mL) directly to reactor at default speed without stirring.
2) Heat/Chill reactor to 25° C. with stirring at 250 RPM. Temperature control is continued after the temperature has been reached.
3) Add benzyl bromide (1.25 mL, 5.1 mmol, 1.02 eq.) directly to reactor at default speed with stirring at 250 RPM
4) Stir reactor for 20 min at 250 RPM stopping stirring afterwards.
5) Heat/Chill reactor to 81° C. for 2 h with stirring at 250 RPM.
6) Heat/Chill reactor to 25° C. with stirring at 250 RPM. Temperature control is continued after the temperature has been reached.
7) Filter contents of reactor through celite to rotavap eluting with MeCN (1×20 mL).
8) Evaporate contents of rotavap with pressure 110 mbar at temperature 30° C. for 30 min.
9) Dry contents of rotavap for 60 min at pressure 303 mbar at temperature 40° C. stopping heating when step finishes.
10) Shut down the platform.

The product N-(4-Bromophenyl)-4-methylbenzenesulfonamide SI-1 (1.1 g, 4.3 mmol, 86%) was obtained as an off-white solid. The recorded NMR signals are in agreement with the literature data.(24)

### 2-Methyl-2-propanyl (4-methoxybenzyl)carbamate SI-2

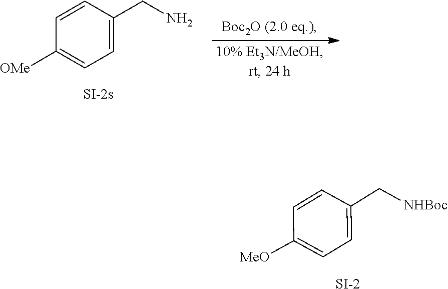

The synthesis was performed following literature protocol by Gellerman et al. (25)

### Manual Preparation

The system was configured as specified in the graph file for this reaction. The reactor was charged with 4-methoxybenzylamine (0.68 g, 5.0 mmol, 1.0 eq.). The solutions of 10% triethylamine in MeOH, t-butoxycarbonyl anhydride (0.33M) in MeOH and 1M potassium hydrogen sulfate were prepared manually.

### ChemPU Steps

1) Add 10% triethylamine in methanol solution (30 mL) directly to reactor at default speed without stirring.
2) Add t-butoxycarbonyl anhydride in MeOH (30 mL, 0.33 M, 10 mmol, 2.0 eq.) directly to reactor at default speed with stirring at 600 RPM.
3) Heat/Chill reactor to 25° C. for 24 h with stirring at 250 RPM.
4) Transfer all from reactor directly to rotavap at default speed, without flushing tubing after the transfer.
5) Evaporate contents of rotavap with pressure 218 mbar at temperature 50° C. for 30 min.
6) Add ethyl acetate (50 mL) directly to rotavap at default speed without stirring.
7) Dissolve contents of rotavap in 1 M potassium hydrogen sulfate (50 mL) at 25° C. over 20 min, stirring at 280 RPM.
8) Extract two-phase mixture in rotavap without adding solvent. Transfer waste phase (bottom) to waste, and product phase (top) directly to separator.
9) Wash contents of separator with water (1×25 mL). Transfer waste phase (bottom) to waste, and product phase (top) through ethyl acetate to reactor.
10) Transfer 200 mL from reactor through magnesium sulfate to rotavap at default speed, without flushing tubing after the transfer.
11) Evaporate contents of rotavap with pressure 150 mbar at temperature 40° C. for 30 min.
12) Shut down the platform.

The product, 2-Methyl-2-propanyl (4-methoxybenzyl)carbamate (1.17 g, 4.4 mmol, 88%) was obtained as a white solid. The recorded NMR signals are in agreement with the literature data. (25)

### (−)-Methyl 2-pyrrolidone-5(R)-carboxylate SI-3

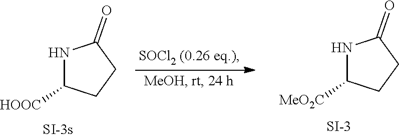

The synthesis was performed following the literature protocol by Westrum et al. (26)

### Manual Preparations

The system was configured as specified in the graph file for this reaction.

(R)-(+)-2-pyrrolidone-5-carboxylic acid (3.30 g, 25.6 mmol) was added manually in the corresponding step.

### ChemPU Steps

1) Add (R)-(+)-2-pyrrolidone-5-carboxylic acid (3.30 g, 25.6 mmol, 1.0 eq.) to reactor.
2) Add methanol (200 mL) directly to reactor at default speed without stirring.
3) Heat/Chill reactor to 25° C. with stirring at 250 RPM. Temperature control is continued after the temperature has been reached.
4) Add thionyl chloride (0.5 mL, 6.9 mmol, 0.26 eq.) directly to reactor over 5 min with stirring at 600 RPM.
5) Stir reactor for 24 h at 250 RPM stopping stirring afterwards.
6) Transfer all from reactor directly to rotavap at default speed, without flushing tubing after the transfer.
7) Add aqueous saturated sodium carbonate (8 mL) directly to rotavap at default speed with stirring at 250 RPM.
8) Evaporate contents of rotavap with pressure 218 mbar at temperature 50° C. for 30 min.
9) Dissolve contents of rotavap in dichloromethane (50 mL) at 25° C. over 20 min, stirring at 400 RPM.
10) Transfer all from rotavap directly to separator at default speed, without flushing tubing after the transfer.
11) Wash contents of separator with aqueous saturated sodium chloride solution (1×10 mL). Transfer waste phase (top) to waste, and product phase (bottom) through anhydrous magnesium sulfate to rotavap.
12) Evaporate contents of rotavap with pressure 218 mbar at temperature 50° C. for 30 min.

The crude product was purified by column chromatography (EtOAc). The product (−)-Methyl 2-pyrrolidone-5(R)-carboxylate SI-3 (2.44 g, 17.1 mmol, 65%) was obtained as a colourless oil.

¹H-NMR (600 MHz, Chloroform-d) δ 6.49 (bs, 1H), 4.25 (dd, 1H, J=9, 5 Hz), 3.76 (s, 3H), 2.55-2.15 (m, 4H). ¹³C-NMR (151 MHz, Chloroform-d) δ 177.5, 172.0, 54.9, 52.2, 28.8, 24.3. ESI-HRMS (M+H)⁺ expected: 144.0655 Da, observed: 144.0698 Da.

### (11bS)-4-hydroxydinaphtho[2,1-d:1′,2′-f][1,3,2]dioxaphosphepine 4-oxide SI-4

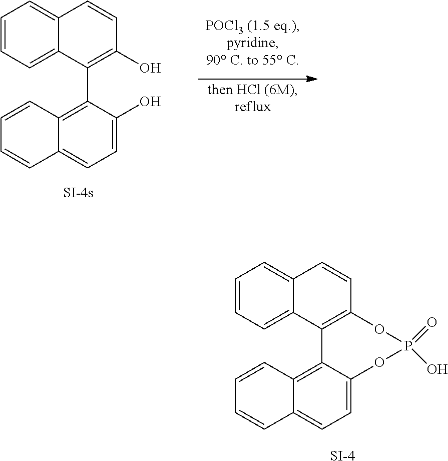

The synthesis was performed following the literature protocol by Jacques and Fouquey. (27)

### Manual Preparations

The system was configured as specified in the graph file. The reactor was charged with of 1,1′-Bi-2-naphthol (10.0 g, 34.9 mmol, 1.0 eq.).

### ChemPU Steps

1) Add pyridine (45 mL) directly to reactor at default speed without stirring.
2) Add phosphorus oxychloride (4.9 mL, 52.5 mmol, 1.50 eq.) directly to reactor at default speed with stirring at 250 RPM.
3) Heat/Chill reactor to 90° C. with stirring at 600 RPM, continue temperature control after the temperature has been reached.
4) Allow to warm/cool reactor to 55° C. with stirring at 600 RPM, stopping temperature control once the temperature has been reached.
5) Add water (4 mL) directly to reactor at default speed with stirring at 250 RPM.
6) Set stir rate to 600 RPM and start stirring filter.
7) Add 6N hydrochloric acid (90 mL) directly to filter at default speed with stirring at 600 RPM.
8) Transfer all from reactor directly to filter at default speed, without flushing tubing after the transfer.
9) Repeat the following 2 items.
   a) Add pyridine (2 mL) directly to reactor at default speed with stirring at 250 RPM.
   b) Transfer 10 mL from the reactor directly to filter at default speed without flushing tubing after the transfer.
10) Filter contents of filter applying vacuum for 2 minutes with stirring at 500 RMP, discarding filtrate using standard transfer speeds.
11) Add 6N hydrochloric acid (30 mL) directly to filter at default speed with stirring 250 RPM.
12) Stir filter for 5 min at 250 RPM, leaving stirring on afterwards.
13) Heat/Chill filter to 108° C. with stirring at 250 RPM, continue temperature control after the temperature has been reached.
14) Heat/Chill filter to 25° C. with stirring at 250 RPM, continue temperature control after the temperature has been reached.
15) Filter contents of filter applying vacuum for 2 minutes with stirring at 500 RMP, discarding filtrate using standard transfer speeds.
16) Wash solid in filter with water (2×20 mL) without temperature control with stirring at 500 RPM, applying vacuum for 2 minutes, discarding filtrate.
17) Dry contents of filter for 1 hr at default pressure without temperature control stopping heating when step finishes.

The product (11bS)-4-hydroxydinaphtho[2,1-d:1′,2′-f][1,3,2]dioxaphosphepine 4-oxide compound with pyridine (71:29) SI-4 (12.7 g, 13.9 mmol, 40% based on ¹H-NMR ratio of the adduct) was obtained as white solid.

The product (11bS)-4-hydroxydinaphtho[2,1-d:1′,2′-d][1,3,2]dioxaphosphepine 4-oxide SI-4 was obtained as a mixture with pyridine (molar ratio=1:0.4) as a white solid (12.7 g, 92% wt., 33.5 mmol, 96%). The recorded NMR signals are in agreement with the literature data. (28)

### Nicotinonitrile SI-5

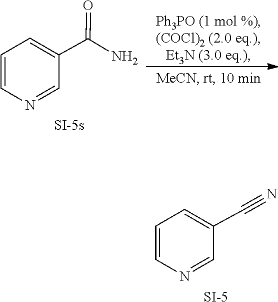

The synthesis was performed according to the modified literature protocol by Malkov et al. (29)

### Manual Preparation

The reactor was charged with nicotinamide SI-5s (2.49 g, 20.0 mmol, 1.0 eq.) and Ph₃PO (56.8 mg, 0.20 mmol, 0.01 eq.).

### ChemPU Steps

1) Dissolve contents of reactor in anhydrous acetonitrile (80 mL) at 25° C. over 10 min, stirring at 250 RPM.
2) Add triethylamine (8.45 mL, 60.0 mmol, 3.00 eq.) directly to reactor at default speed with stirring at 250 RPM.
3) Add oxalyl chloride (3.50 mL, 40.0 mmol, 2.00 eq.) directly to reactor over 30 min with stirring at 250 RPM.
4) Heat/Chill reactor to 25° C. for 2 h with stirring at 250 RPM.
5) Transfer all from reactor directly to rotavap at default speed, without flushing tubing after the transfer.
6) Evaporate contents of rotavap with default pressure control at temperature 50° C. for 30 min.
7) Dry contents of rotavap for 30 min at pressure 50 mbar at temperature 50° C. stopping heating when step finishes.

The product nicotinonitrile SI-5 was found to resublimate readily. Thus, the yield of the reaction (98%) was determined by ¹H-NMR analysis of the crude product using 1,3,5-trimethoxybenzene as an internal standard.

The crude product was purified by flash column chromatography (100 g Biotage ‘Sfär’ silica column, 60 μm particle size, 100 Å pore size, spherical particle shape) using a gradient of ethyl acetate in petroleum ether (10%-50% over 15 column volumes). An analytically pure sample was obtained after resublimation at 50° C. and 15 mbar. The recorded NMR signals are in agreement with the literature data. (29)

### 3-Oxocholic acid SI-6

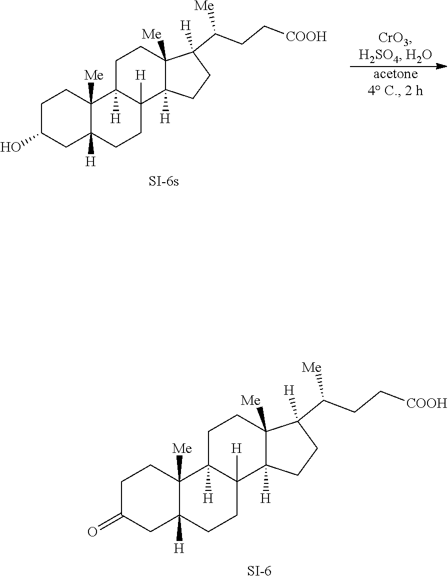

The synthesis was performed according to the modified literature protocol by Chen et al. (30)

### Manual Preparation

The reactor was charged with lithocholic acid SI-6s (535 g, 1.35 mmol, 1.0 eq.) and the Jones reagent flask was charged with CrO₃ (3.03 g, 30.0 mmol).

### ChemPU Steps

1) Dissolve contents of reactor in acetone (50 mL) at 25° C. over 20 min, stirring at 400 RPM.
2) Dissolve contents of flask ‘Jones_reagent’ in water (9 mL) at 25° C. over 5 min, stirring at 400 RPM.
3) Add H₂SO₄ (2.97 mL) directly to flask ‘Jones_reagent’ at default speed with stirring at 250 RPM.
4) Heat/Chill reactor to 4° C. with stirring at 250 RPM. Temperature control is continued after the temperature has been reached.
5) Add Jones reagent (5 mL, 2.51 M, 12.5 mmol, 9.28 eq.) directly to reactor at default speed with stirring at 250 RPM.
6) Heat/Chill reactor to 4° C. for 4 h with stirring at 250 RPM.
7) Heat/Chill reactor to 25° C. with stirring at 250 RPM. Temperature control is continued after the temperature has been reached.
8) Transfer all from reactor directly to rotavap at default speed, without flushing tubing after the transfer.
9) Repeat 5 times:
   a) Add acetone (30 mL) directly to reactor at default speed with stirring at 250 RPM.
   b) Heat/Chill reactor to 25° C. for 5 min with stirring at 250 RPM.
   c) Transfer all from reactor directly to rotavap at default speed, without flushing tubing after the transfer.
10) Evaporate contents of rotavap with default pressure control at temperature 45° C. for 30 min.
11) Dry contents of rotavap for 30 min at pressure 50 mbar at temperature 45° C. stopping heating when step finishes.
12) Repeat 3 times:
   a) Add diethyl ether (30 mL) directly to rotavap at default speed with stirring at 250 RPM.
   b) Stir rotavap for 2 min at 250 RPM stopping stirring afterwards.
   c) Transfer all from rotavap directly to separator at default speed, without flushing tubing after the transfer.
13) Repeat 3 times:
   a) Add water (15 mL) directly to rotavap at default speed with stirring at 250 RPM.
   b) Stir rotavap for 2 min at 250 RPM stopping stirring afterwards.
   c) Transfer all from rotavap directly to separator at default speed, without flushing tubing after the transfer.
14) Repeat 3 times:
   a) Add water (15 mL) directly to reactor at default speed with stirring at 250 RPM.
   b) Stir reactor for 2 min at 250 RPM stopping stirring afterwards.
   c) Transfer all from reactor directly to separator at default speed, without flushing tubing after the transfer.
15) Repeat 3 times:
   a) Add diethyl ether (15 mL) directly to reactor at default speed with stirring at 250 RPM.
   b) Stir reactor for 2 min at 250 RPM stopping stirring afterwards.
   c) Transfer all from reactor directly to separator at default speed, without flushing tubing after the transfer.
16) Clean rotavap with water (2× solvent volume 50 mL) without temperature control, without drying, stirring for 60 s at.
17) Clean rotavap with acetone (2× solvent volume 50 mL) without temperature control, with drying if possible, stirring for 60 s at.
18) Add HCl (5 mL) directly to separator at default speed without stirring.
19) Separate mixture in separator, sending lower phase directly to buffer_flask1 and upper phase directly to buffer_flask2.
20) Transfer 250 mL from buffer_flask1 directly to separator at default speed, without flushing tubing after the transfer.
21) Extract contents of separator with diethyl ether (2×25 mL). Transfer waste phase (bottom) to waste, and product phase (top) directly to buffer_flask2.
22) Transfer 300 mL from buffer_flask2 directly to separator at default speed, without flushing tubing after the transfer.
23) Wash contents of separator with brine (1×20 mL). Transfer waste phase (bottom) to waste, and product phase (top) through MgSO₄ to rotavap.
24) Evaporate contents of rotavap with default pressure control at temperature 45° C. for 30 min.
25) Dry contents of rotavap for 1.5 h at pressure 5 mbar at temperature 75° C. stopping heating when step finishes.
26) Clean flaskJones_reagent with water (2× solvent volume 100 mL) at temperature 24° C., with drying if possible, stirring for 60 s at.
27) Clean flask Jones_reagent with acetone (2× solvent volume 100 mL) at temperature 24° C., with drying if possible, stirring for 60 s at.
28) Clean flask ‘Jones_reagent’ with water (1× solvent volume 100 mL) at temperature 24° C., with drying if possible, stirring for 60 s at.
29) Clean reactor with water (1× solvent volume 100 mL) at temperature 24° C., without drying, stirring for 60 s at.
30) Clean reactor with acetone (2× solvent volume 100 mL) at temperature 24° C., with drying if possible, stirring for 60 s.

The product 3-oxocholic acid SI-6 was obtained as a pale yellow solid (0.326 g, 0.871 mmol, 65% yield). The recorded NMR signals are in agreement with the literature data. (30)

### Sodium (Z)-1-cyanoprop-1-en-2-olate SI-7

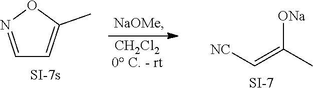

The synthesis was performed following the literature protocol by Carreira et al. (31)

### Manual Preparation

The system was configured as specified in the graph file for this reaction.

### ChemPU Steps

1) Add 5-methylisoxazole (3.0 mL, 35.5 mmol, 1.08 eq.) directly to reactor at default speed without stirring.
2) Dissolve contents of reactor in CH₂Cl₂ (42 mL) at 25° C. over 20 min, stirring at 400 RPM.
3) Heat/Chill reactor to 0° C. with stirring at 250 RPM. Temperature control is continued after the temperature has been reached.
4) Add NaOMe (6.6 mL, 5.0 M in methanol, 33 mmol, 1 eq.) directly to reactor at default speed with stirring at 250 RPM.
5) Allow to warm/cool reactor to 18° C. with stirring at 250 RPM. Temperature control is stopped once the temperature has been reached.
6) Stir reactor for 16 h at 250 RPM stopping stirring afterwards.
7) Transfer all from reactor directly to rotavap at default speed, without flushing tubing after the transfer.
8) Evaporate contents of rotavap with pressure 708 mbar at temperature 50° C. for 30 min.
9) Wash solid in rotavap with anhydrous Et₂O (1×20 mL) without temperature control, with stirring at 500 RPM, applying vacuum for 10 s, discarding filtrate.

The product sodium (2)-1-cyanoprop-1-en-2-olate SI-7 (2.60 g, 24.8 mmol, 75%) was obtained as an off-white solid. The recorded NMR signals are in agreement with the literature data. (32)

### 2-Methoxy-6-(piperidin-1-yl)pyridine SI-8

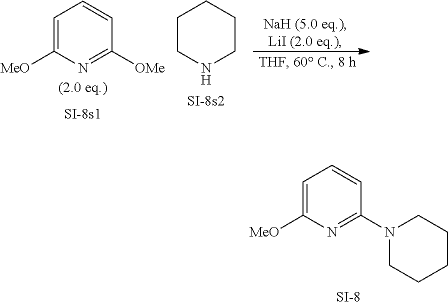

The synthesis was performed following the literature protocol by Chiba et al. (33)

### Manual Preparation

Sodium hydride (60% suspension in mineral oil) and lithium iodide were dried in vacuo at 120° C. over P₂O₅ for 2 days prior to use. A solution of 2,6-dimethoxypyridne SI-8s1 (13.5 mL) in dry THF (total volume 25 mL, 4.09 M) and a solution of piperidine SI-8s2 (5.0 mL) in dry THF (total volume 25 mL, 2.02 M) were prepared. The reactor was charged with sodium hydride (2.00 g, 50.0 mmol, 5.0 eq.) and lithium iodide (2.70 g, 20.0 mmol, 2.0 eq.).

### ChemPU Steps

1) Dry contents of reactor for 60 min at pressure 10 mbar at temperature 140° C. stopping heating when step finishes.
2) Heat/Chill reactor to 25° C. with stirring at 250 rpm. Temperature control is continued after the temperature has been reached.
3) Evacuate reactor and refill with inert gas 3 times, using a vacuum pressure of 10 mbar, waiting 60 s after evacuating and 60 s after refilling with inert gas.
4) Add THF (3.7 mL) directly to reactor at default speed with stirring at 250 rpm.
5) Add methoxypyridine in THF (5.0 mL, 4.09 M, 20.4 mmol, 2.0 eq.) directly to reactor at default speed with stirring at 250 rpm.
6) Add piperidine in THF (5 mL, 2.02 M, 10.1 mmol, 1.0 eq.) directly to reactor at default speed with stirring at 250 rpm.
7) Heat/Chill reactor to 75° C. for 8 h with stirring at 250 rpm.
8) Heat/Chill reactor to 0° C. with stirring at 250 rpm. Temperature control is continued after the temperature has been reached.
9) Add water (50 mL) directly to reactor over 10 min with stirring at 250 rpm.
10) Heat/Chill reactor to 25° C. with stirring at 250 rpm. Temperature control is continued after the temperature has been reached.
11) Add DCM (20 mL) directly to reactor at default speed without stirring.
12) Transfer all from reactor directly to separator at default speed, without flushing tubing after the transfer.
13) Add DCM (30 mL) directly to reactor at default speed without stirring.
14) Transfer all from reactor directly to separator at default speed, without flushing tubing after the transfer.
15) Extract contents of separator with DCM (1×50 mL). Transfer waste phase (top) to vessel separator, and product phase (bottom) through MgSO₄ to rotavap.
16) Extract contents of separator with DCM (3×100 mL). Transfer waste phase (top) to waste, and product phase (bottom) through MgSO₄ to rotavap.
17) Evaporate contents of rotavap with pressure 699 mbar at temperature 50° C. for 30 min.
18) Dry contents of rotavap for 30 min at pressure 10 mbar at temperature 50° C. stopping heating when step finishes.
19) Shut down the platform.

The crude product was purified by flash column chromatography (50 g Biotage ‘Sfär’ silica column, 60 μm particle size, 100 Å pore size, spherical particle shape) using a gradient of toluene in petroleum ether (0%-50% over 15 column volumes).

The product 2-methoxy-6-(piperidin-1-yl)pyridine SI-8 (2.26 g, 5.32 mmol, 53%) was obtained as a pale yellow oil. The recorded NMR signals are in agreement with the literature data. (33)

### N-(4-(Trifluoromethyl)phenyl)pyrimidin-5-amine SI-9

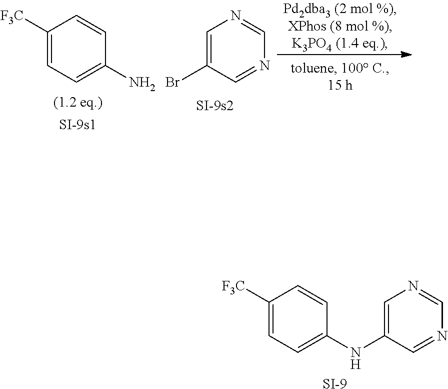

The synthesis was performed following the literature protocol by Buchwald et al. (34)

### Manual Preparations

The reactor was charged with 4-(trifluoromethyl)aniline SI-9s1 (0.77 mL, 6.0 mmol, 1.2 eq.), 5-bromopyridine SI-9s2 (803 mg, 5.00 mmol, 1.0 eq.), Pd₂dba₃ (92.5 mg, 0.100 mmol, 0.02 eq.), XPhos (193 mg, 0.400 mmol, 0.08 eq.) and K₃PO₄ (1.50 g, 7.00 mmol, 1.4 eq.).

### ChemPU Steps

1) Evacuate reactor and refill with inert gas 3 times, using a vacuum pressure of 10 mbar, waiting 60 s after evacuating and 60 s after refilling with inert gas.
2) Add toluene (10 mL) directly to reactor at default speed without stirring.
3) Heat/Chill reactor to 110° C. for 15 h with stirring at 250 rpm.
4) Transfer 50 mL from reactor through Celite to rotavap at default speed, without flushing tubing after the transfer.
5) Repeat 3 times:
   a) Add ethyl acetate (50 mL) directly to reactor at default speed without stirring.
   b) Transfer 150 mL from reactor through Celite to rotavap at default speed, without flushing tubing after the transfer.
6) Evaporate contents of rotavap with default pressure control at temperature 50° C. for 30 min.
7) Dry contents of rotavap for 30 min at pressure 10 mbar at temperature 50° C. stopping heating when step finishes.
8) Shut down the platform.

The crude product was purified by flash column chromatography (25 g Biotage ‘Sfär’ silica column, 60 μm particle size, 100 Å pore size, spherical particle shape) using a gradient of ethyl acetate in petroleum ether (50%-100% over 10 column volumes).

The product N-(4-(trifluoromethyl)phenyl)pyrimidin-5-amine SI-9 (894 mg, 3.74 mmol, 75%) was obtained as a white solid. The recorded NMR signals are in agreement with the literature data. (34)

### Ethyl 3-(hydroxymethyl)but-3-enoate SI-10

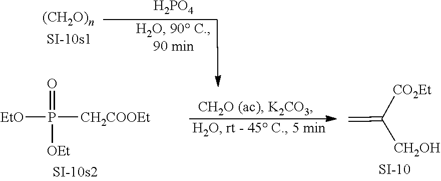

The synthesis was performed following the literature protocol by Villieras and Rambaud. (35)

### Manual Preparations

The reactor was charged with solid paraformaldehyde SI-10s1 (12.0 g, 0.4 mol, 4 eq.). The drying cartridge was charged with fresh MgSO₄ and sand mixture (5 g, 1:1 mass).

### ChemPU Steps

1) Add water (27.5 mL) directly to reactor at default speed without stirring.
2) Add 1 N phosphoric acid (1 mL) directly to reactor at default speed with stirring at 250 RPM.
3) Heat/Chill reactor to 90° C. for 1.5 h with stirring at 250 RPM.
4) Heat/Chill reactor to 25° C. with stirring at 250 RPM. Temperature control is continued after the temperature has been reached.
5) Add triethyl phosphonoacetate SI-10s2 (20 mL, 0.1 mol, 1 eq.) directly to reactor at default speed with stirring at 250 RPM.
6) Stir reactor for 5 min at 250 RPM stopping stirring afterwards.
7) Heat/Chill reactor to 35° C. with stirring at 250 RPM. Temperature control is continued after the temperature has been reached.
8) Add potassium carbonate water solution (15 mL, 7.3 M, 0.11 mol, 1.1 eq.) directly to reactor over 60 min with stirring at 500 RPM.
9) Stir reactor for 5 min at 250 RPM stopping stirring afterwards.
10) Heat/Chill reactor to 20° C. with stirring at 250 RPM. Temperature control is continued after the temperature has been reached.
11) Add diethyl ether (50 mL) directly to reactor at default speed with stirring at 250 RPM.
12) Add brine (37.5 mL) directly to reactor at default speed with stirring at 250 RPM.
13) Extract contents of reactor with diethyl ether (3×25 mL). Transfer waste phase (bottom) to waste, and product phase (top) directly to separator.
14) Wash contents of separator with brine (2×25 mL). Transfer waste phase (bottom) to waste, and product phase (top) through magnesium sulfate to rotavap.
15) Evaporate contents of rotavap with pressure 440 mbar at temperature 50° C. for 30 min.

### 16) Distill

Crude ethyl 2-(diethoxyphosphoryl)acetate SI-10 (11.46 g, 67% purity, average crude yield over 3 experiments—61%) was obtained as a yellow oil. The material was distilled under vacuum (80-83° C. @7 mbar) to afford pure SI-10 (1.51 g, 3.57 mmol, 36%) as colourless oil.

The recorded NMR signals are in agreement with the literature data. (35)

### 2-Hydroxy-2,2-diphenylacetonitrile SI-11

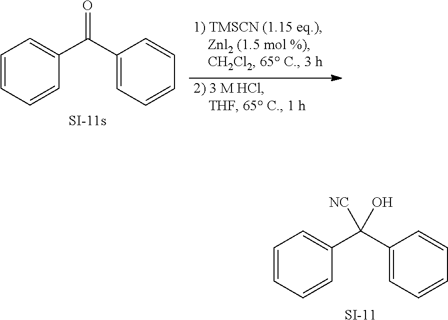

The synthesis was performed following the literature protocol by Paul G. Gassman and John J. Talley. (36)

### Manual Preparations

The system was configured as specified in the graph file for this reaction. The reagent flask ‘benzophenone’ was charged with a weighed amount of benzophenone (3.64 g, 20.0 mmol, 1.00 eq.) and the reagent flask ‘tmscn’ was charged with trimethylsilyl cyanide (2.28 g, 2.87 mL, 23.0 mmol, 1.15 eq.). Zinc iodide (95.7 mg, 0.3 mmol, 1.5 mol %) was added manually in the corresponding step.

### ChemPU Steps

1) Add zinc iodide (95.7 mg, 0.3 mmol, 1.5 mol %) to reactor.
2) Repeat 2 times:
   a) Add CH₂Cl₂ (2 mL) directly to tmscn at default speed without stirring.
   b) Transfer all from tmscn directly to reactor at default speed, without flushing tubing after the transfer.
3) Repeat 2 times:
   a) Add CH₂Cl₂ (3 mL) directly to benzophenone at default speed without stirring.
   b) Transfer all from benzophenone directly to reactor at default speed, without flushing tubing after the transfer.
4) Add CH₂Cl₂ (2 mL) directly to reactor at default speed with stirring at 250 RPM.
5) Heat/Chill reactor to 65° C. for 3 h with stirring at 250 RPM.
6) Allow to warm/cool reactor to 30° C. with stirring at 250 RPM. Temperature control is stopped once the temperature has been reached.
7) Evaporate contents of reactor with pressure 20 mbar at temperature 40° C. for 60 min.
8) Add tetrahydrofuran (10 mL) directly to reactor at default speed with stirring at 250 RPM.
9) Add 3M HCl (6 mL) directly to reactor at default speed with stirring at 250 RPM.
10) Heat/Chill reactor to 65° C. for 60 min with stirring at 250 RPM.
11) Allow to warm/cool reactor to 30° C. with stirring at 250 RPM. Temperature control is stopped once the temperature has been reached.
12) Evaporate contents of reactor with pressure 20 mbar at temperature 40° C. for 60 min.
13) Repeat 2 times:
   a) Add water (15 mL) directly to reactor at default speed with stirring at 250 RPM.
   b) Add diethylether (15 mL) directly to reactor at default speed with stirring at 250 RPM.
   c) Transfer all from reactor directly to separator at default speed, without flushing tubing after the transfer.
14) Extract contents of separator with diethylether (3×30 mL). Transfer waste phase (bottom) to waste, and product phase (top) directly to separator.
15) Wash contents of separator with NaCl (1×30 mL). Transfer waste phase (bottom) to waste, and product phase (top) directly to separator.
16) Transfer all from separator through MgSO₄ to rotavap at default speed, without flushing tubing after the transfer.
17) Evaporate contents of rotavap with pressure 50 mbar at temperature 40° C. for 60 min.
18) CConnect
19) Repeat 2 times:
   a) Add diethylether (20 mL) directly to rotavap at default speed with stirring at 250 RPM.
   b) Stop stirring rotavap.
   c) Transfer all from rotavap directly to filter at default speed, without flushing tubing after the transfer.
20) CConnect
21) Evaporate contents of filter with pressure 50 mbar at temperature 30° C. for 45 min.
22) CConnect
23) Recrystallize contents of filter by dissolving in toluene (40 mL) at 80° C. heating to 10° C. and waiting for 3 h.
24) Filter contents of filter, without stirring, discarding filtrate using standard transfer speeds.

The product 2-hydroxy-2,2-diphenylacetonitrle SI-11 (2.02 mg, 9.65 mmol, 48%) was obtained as a white solid. A second crop of crystals (646 mg, 3.09 mmol, 16%) was obtained by concentrating the recrystallization filtrate. The recorded NMR signals are in agreement with the literature data. (36)

### 1,1,1-trifluoro-2-(4-fluorophenyl)propan-2-ol SI-12

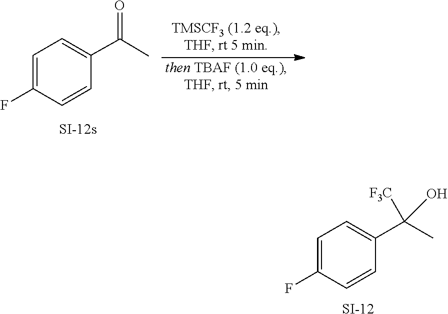

The synthesis was performed following the literature protocol by Lloyd-Jones et al. (37)

### Manual Preparations

THF solutions of 4-fluoroacetophenone SI-12s (0.4 M), trifluoromethylsilane (0.48M), and TBAF (0.1M and 1.0 M) were prepared.

### ChemPU Steps

1) Add 4′-fluoroacetophenone SI-12s (10 mL, 0.4 M in THF, 4.0 mmol, 1.0 eq.) directly to reactor at default speed without stirring.
2) Add trifluoromethylsilane (10 mL, 0.48M in THF, 4.8 mmol, 1.2 eq.) directly to reactor at default speed with stirring at 250 RPM.
3) Add TBAF (0.4 mL, 0.1 M in THF, 0.04 mmol, 0.01 eq.) directly to reactor at default speed with stirring at 250 RPM.
4) Stir reactor for 5 min at 250 RPM stopping stirring afterwards.
5) Add TBAF (4.0 mL, 1.0 M in THF, 4.0 mmol, 1.0 eq.) directly to reactor at default speed without stirring.
6) Stir reactor for 5 min at 250 RPM stopping stirring afterwards.
7) Transfer all from reactor directly to rotavap at default speed, without flushing tubing after the transfer.
8) Evaporate contents of rotavap with pressure 100 mbar at temperature 25° C. for 30 min.

The yield (>99%) was determined using ¹⁹F-NMR analysis of the crude reaction mixture with 1,4-difluorobenzene as an internal standard. The NMR was on the crude material obtained after reaction. The recorded NMR signals are in agreement with the literature data. (37)

### 2-phenyl-thiophene-5-boronic acid MIDA ester SI-13

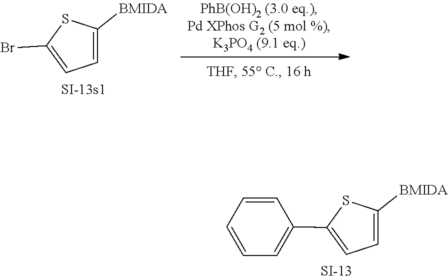

The synthesis was performed following the literature protocol by Watson et al. (2)

### Manual Preparations

The system was configured as specified in the graph file for this reaction. Potassium phosphate (5.10 g, 24.0 mmol, 9.10 eq.), XPhos Pd G2 catalyst (104 mg, 0.132 mmol, 0.05 eq.), bifunctional MIDA boronate SI-13s1 (840 mg, 2.64 mmol, 1.0 eq.), and phenyl boronic acid (975 mg, 8.00 mmol, 3.0 eq.) were added manually in the corresponding steps.

### ChemPU Steps

1) Add potassium phosphate (5.10 g, 24.0 mmol, 9.10 eq.) to reactor.
2) Add bifunctional MIDA boronate SI-13s1 (840 mg, 2.64 mmol, 1.0 eq.) to reactor. 3) Add XPhos Pd G2 (104 mg, 0.132 mmol, 0.05 eq.) to reactor.
4) Switch pneumatic controller to vacuum for flask_holding and wait for 30 s.
5) Switch pneumatic controller to low pressure inert gas for flask_holding and wait for 30 s.
6) Switch pneumatic controller to vacuum for flask_holding and wait for 30 s.
7) Switch pneumatic controller to low pressure inert gas for flask_holding and wait for 30 s.
8) Switch pneumatic controller to vacuum for flask_holding and wait for 30 s.
9) Switch pneumatic controller to low pressure inert gas for flask_holding and wait for 30 s.
10) Add phenyl boronic acid (975 mg, 8.00 mmol, 3.0 eq.) to flask_holding.
11) Transfer 125 mL from argon directly to waste_7 at default speed, without flushing tubing after the transfer.
12) Dissolve contents of flask_holding in THF (20 mL) at 25° C. over 20 min, stirring at 400 RPM.
13) Switch pneumatic controller to vacuum for flask_holding and wait for 30 s.
14) Switch pneumatic controller to low pressure inert gas for flask_holding and wait for 30s.
15) Switch pneumatic controller to vacuum for flask_holding and wait for 30 s.
16) Switch pneumatic controller to low pressure inert gas for flask_holding and wait for 30s.
17) Switch pneumatic controller to vacuum for flask_holding and wait for 30 s.
18) Switch pneumatic controller to low pressure inert gas for flask_holding and wait for 30s.
19) Switch pneumatic controller to vacuum for reactor and wait for 30 s.
20) Switch pneumatic controller to low pressure inert gas for reactor and wait for 30 s.
21) Switch pneumatic controller to vacuum for reactor and wait for 30 s.
22) Switch pneumatic controller to low pressure inert gas for reactor and wait for 30 s.
23) Switch pneumatic controller to vacuum for reactor and wait for 30 s.
24) Switch pneumatic controller to low pressure inert gas for reactor and wait for 30 s.
25) Add THF (28 mL) directly to reactor at default speed with stirring at 250 RPM.
26) Switch pneumatic controller to vacuum for reactor and wait for 30 s.
27) Switch pneumatic controller to low pressure inert gas for reactor and wait for 30 s.
28) Switch pneumatic controller to vacuum for reactor and wait for 30 s.
29) Switch pneumatic controller to low pressure inert gas for reactor and wait for 30 s.
30) Switch pneumatic controller to vacuum for reactor and wait for 30 s.
31) Switch pneumatic controller to low pressure inert gas for reactor and wait for 30 s.
32) Heat/Chill reactor to 55° C. with stirring at 250 RPM. Temperature control is continued after the temperature has been reached.
33) Transfer all from flask_holding directly to reactor over 4 h, without flushing tubing after the transfer.
34) Repeat 2 times:
   a) Add THF (5 mL) directly to flask_holding at default speed with stirring at 250 RPM.
   b) Transfer 5 mL from flask_holding directly to reactor at default speed, without flushing tubing after the transfer.
35) Stir reactor for 12 h at 250 RPM stopping stirring afterwards.
36) Set stir rate to 50 RPM and start stirring reactor.
37) Transfer 100 mL from reactor directly to rotavap at default speed, without flushing tubing after the transfer.
38) Repeat 2 times:
   a) Add THF (10 mL) directly to reactor at default speed with stirring at 250 RPM.
   b) Transfer 100 mL from reactor directly to rotavap at default speed, without flushing tubing after the transfer.
39) Evaporate contents of rotavap with pressure 249 mbar at temperature 50° C. for 30 min.

The crude material was purified by flash column chromatography (0-10% methanol in dichloromethane).

The product 2-phenyl-thiophene-5-boronic acid MIDA ester SI-13 (572 mg, 1.82 mmol, 69%) was obtained as a light brown solid. The recorded NMR signals are in agreement with the literature data. (2)

### N-allyl-N-(1-(4-chlorophenyl)-2-(cyclohexylamino)-2-oxoethyl)-2-iodobenzamide 9d

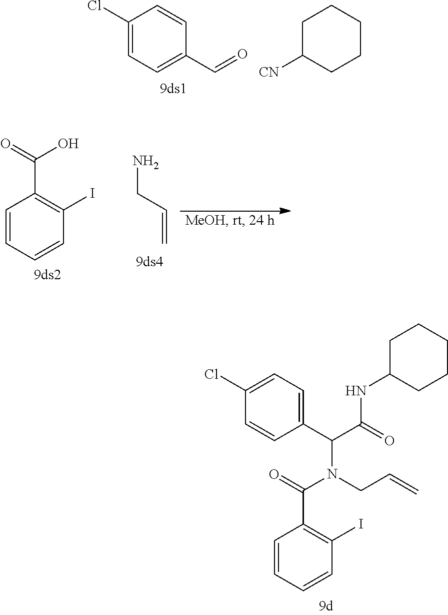

The synthesis was performed following the literature protocol by Xiang et al. (14)

### Manual Preparations

The system was configured as specified in the graph file for this reaction.1M methanol stock solutions of allylamine 9ds4 and 2-iodobenzoic acid 9ds2 were prepared prior to experiment. The filter was charged with solid 4-chlorobenzaldehyde 9ds1 (0.84 g, 6.00 mmol, 1 eq.). Cyclohexyl isocyanide 9ds3 (0.65 g, 6.0 mmol, 1.0 eq.) was added manually in the corresponding step.

### ChemPU Steps

1) Fill bottom of filter with nitrogen gas (10 mL).
2) Add allylamine 9ds4 methanol solution (1 M) (6 mL) directly to filter at default speed with stirring at 120 rpm.
3) Stir filter for 10 min at 120 rpm stopping stirring afterwards.
4) Add 2-iodobenzoic acid 9ds2 methanol solution (1 M) (6 mL) directly to filter at default speed with stirring at 120 rpm.
5) Stir filter for 10 min at 120 rpm stopping stirring afterwards.
6) Add cyclohexyl isocyanide 9ds3 (0.65 g, 6.0 mmol, 1.0 eq.) directly to flask_isocyanide at default speed manually.
7) Add methanol (8 mL) directly to flask_isocyanide at default speed with stirring at 450 rpm.
8) Transfer all from flask_isocyanide directly to filter at default speed, flushing tubing with methanol after the transfer
9) Stir filter for 1 min at 150 rpm stopping stirring afterwards.
10) Wait for 24 h.
11) Transfer 50 mL from filter directly to waste at default speed, without flushing tubing after the transfer.

The product N-allyl-N-(1-(4-chlorophenyl)-2-(cyclohexylamino)-2-oxoethyl)-2-iodobenzamide 9d (1.82 g, 3.39 mmol, 56%) was obtained as a white solid. The recorded NMR signals are in agreement with the literature data. (14)

### 2-(4-chlorophenyl)-N-cyclohexyl-2-(4-methyl-1-oxoisoquinolin-2(1H)-yl)acetamide SI-14

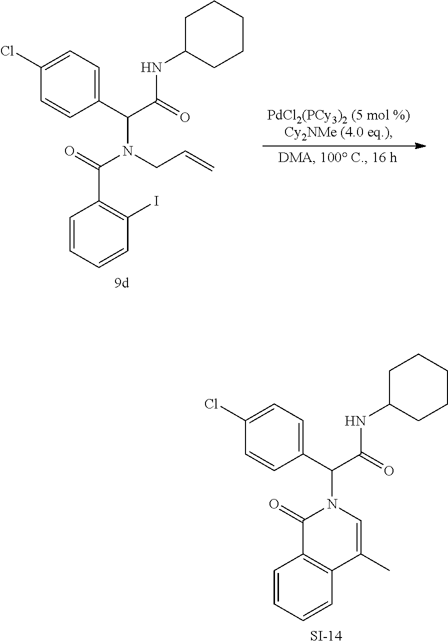

The synthesis was performed following the literature protocol by Xiang et al. (14)

### Manual Preparations

The system was configured as specified in the graph file for this reaction. The filter was charged with solid intermediate 9d (1.00 g, 1.86 mmol, 1.00 eq.). PdCl₂(PCy₃)₂ (0.07 g, 0.90 mmol, 0.05 eq.) was added manually in the corresponding step.

### ChemPU Steps

1) Fill bottom of filter with nitrogen gas (10 mL).
2) Add dimethylacetamide (20 mL) directly to filter at default speed with stirring at 150 rpm.
3) Add PdCl₂(PCy₃), (0.07 g, 0.09 mmol, 0.05 eq.) to filter.
4) Add N-methyldicyclohexylamine (1.60 mL, 7.45 mmol, 4.00 eq.) directly to filter at default speed with stirring at 150 RPM
5) Heat/Chill reactor to 100° C. For 16 h with stirring at 150 rpm.
6) Transfer 50 ml from filter directly to rotavap at default speed, flushing tubing with dimethylacetamide after the transfer.
7) Evaporate contents of rotavap with pressure 10 mbar at temperature 55° C. For 2 h.

The crude product was purified by flash column chromatography (10 g Biotage ‘Sfär’ silica column, 60 μm particle size, 100 Å pore size, spherical particle shape) using a gradient of ethyl acetate in n-hexane (25%-100% over 10 column volumes).

The product 2-(4-chlorophenyl)-N-cyclohexyl-2-(4-methyl-1-oxoisoquinolin-2(1H)-yl)acetamide SI-14 (0.24 g, 0.59 mmol, 32%) was obtained as a white solid. The recorded NMR signals are in agreement with the literature data. (14)

### 2-(4-chlorophenyl)-N-cyclohexyl-2-(4-methyl-1-oxoisoquinolin-2(1H)-yl)acetamide SI-14

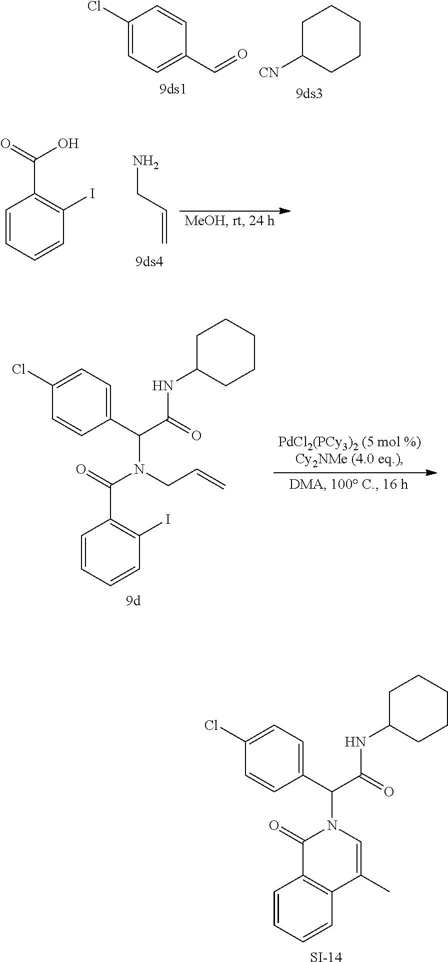

The synthesis was performed following the literature protocol by Xiang et al. (14)

### Manual Preparations

The system was configured as specified in the graph file for this reaction.1M methanol stock solutions of allylamine 9ds4 and 2-iodobenzoic acid 9ds2 were prepared prior to experiment. The filter was charged with solid 4-chlorobenzaldehyde 9ds1 (0.84 g, 6.0 mmol, 1.0 eq.). Cyclohexyl isocyanide 9ds3 (0.65 g, 6.0 mmol, 1.0 eq.) And PdCl₂(PCy₃)₂ (0.22 g, 0.30 mmol, 0.05 eq.) Were added manually in the corresponding steps.

### ChemPU Steps

1) Fill bottom of filter with nitrogen gas (10 ml).
2) Add allylamine 9ds4 methanol solution (1 M) (6 ml) directly to filter at default speed with stirring at 250 rpm.
3) Stir filter for 10 min at 250 rpm stopping stirring afterwards.
4) Add 2-iodobenzoic acid 9ds2 methanol solution (1 M) (6 ml) directly to filter at default speed with stirring at 250 rpm.
5) Stir filter for 10 min at 250 rpm stopping stirring afterwards.
6) Add cyclohexyl isocyanide 9ds3 (0.65 g, 6.0 mmol, 1.0 eq.) To flask_isocyanide.
7) Add methanol (8 ml) directly to flask_isocyanide at default speed with stirring at 250 rpm.
8) Transfer all from flask_isocyanide directly to filter at default speed, flushing tubing with methanol after the transfer
9) Stir filter for 1 min at 250 rpm stopping stirring afterwards.
10) Wait for 24 h.
11) Transfer 50 ml from filter directly to waste at default speed, without flushing tubing after the transfer.
12) Fill bottom of filter with nitrogen gas (10 ml).
13) Add dimethylacetamide (20 ml) directly to filter at default speed with stirring at 250 rpm.
14) Add PdCl₂(PCy₃)₂ (0.22 g, 0.30 mmol, 0.05 eq.) To filter.
15) Add N-methyldicyclohexylamine (5.14 ml, 24.0 mmol, 4.00 eq.) directly to filter at default speed with stirring at 250 RPM
16) Heat/Chill reactor to 100° C. For 16 h with stirring at 250 rpm.
17) Transfer 50 ml from filter directly to rotavap at default speed, flushing tubing with dimethylacetamide after the transfer.
18) Evaporate contents of rotavap with pressure 10 mbar at temperature 55° C. For 2 h.

The crude product was purified by flash column chromatography (10 g Biotage ‘sfär’ silica column, 60 μm particle size, 100 Å pore size, spherical particle shape) using a gradient of ethyl acetate in n-hexane (25%-100% over 10 column volumes).

The product 2-(4-chlorophenyl)-N-cyclohexyl-2-(4-methyl-1-oxoisoquinolin-2(1H)-yl)acetamide SI-14 (0.86 g, 2.1 mmol, 35%) was obtained as a white solid. The recorded NMR signals are in agreement with the literature data. (14)

### (E)-2-(dimethyl(styryl)silyl)pyridine SI-15a

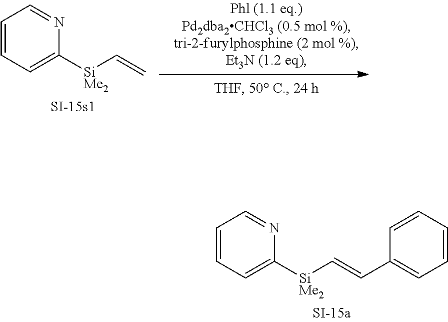

The synthesis was performed following the literature protocol by Yoshida et al. (38)

### Manual Preparation

A solution of 2-pyridyldimethyl(vinyl)silane SI-15s1 (631 mg) in dry THF (total volume 5 ml, 0.773 M), a solution of iodobenzene (0.75 ml) in dry THF (total volume 5 mL, 1.35 M) and a solution of triethylamine (1.01 ml) in THF (total volume 5 ml, 1.45 M) were prepared. The reactor was charged with Pd₂dba₃CHCl₃ (15.7 mg, 0.015 mmol, 0.005 eq.) And tri-2-furylphosphine (14.1 mg, 0.060 mmol, 0.02 eq.).

### ChemPU Steps

1) Evacuate reactor and refill with inert gas 3 times, using a vacuum pressure of 10 mbar, waiting 60 s after evacuating and 60 s after refilling with inert gas.
2) Add iodobenzene solution in THF (2.50 ml, 1.35 M, 3.38 mmol, 1.1 eq.) directly to reactor at default speed with stirring at 250 rpm.
3) Add 2-pyridyldimethyl(vinyl)silane SI-15s1 solution in THF (4.00 ml, 0.773 M, 3.10 mmol, 1.00 eq.) directly to reactor at default speed with stirring at 250 rpm.
4) Add triethylamine solution in THF (2.5 ml, 1.45 M, 3.60 mmol, 1.16 eq.) directly to reactor at default speed with stirring at 250 rpm.
5) Heat/Chill reactor to 57° C. For 24 h with stirring at 250 rpm.
6) Transfer all from reactor directly to separator at default speed, without flushing tubing after the transfer.
7) Repeat 3 times:
   a) Dissolve contents of reactor in toluene (10 ml) at 25° C. Over 60 s, stirring at 400 Rpm.
   b) Transfer 100 ml from reactor directly to separator at default speed, without Flushing tubing after the transfer.
8) Extract contents of separator with 1 M aqueous HCl (4×20 ml). Transfer waste phase (top) to waste, and product phase (bottom) directly to separator.
9) Add sat. Aq. NaHCO₃ (120 ml) directly to separator over 10 min with stirring at 250 rpm.
10) Extract contents of separator with ethyl acetate (3×60 ml). Transfer waste phase (top) to waste, and product phase (bottom) through MgSO₄ to rotavap.
11) Evaporate contents of rotavap with default pressure control at temperature 50° C. For 30 min.
12) Dry contents of rotavap for 60 min at pressure 10 mbar at temperature 50° C. Stopping Heating when step finishes.
13) Shut down the platform.

The product (E)-2-(dimethyl(styryl)silyl)pyridine SI-15a was obtained as a pale yellow solid (467 mg, 1.95 mmol, 63%). The recorded NMR signals are in agreement with the literature data. (38)

### 1-Dimethyl(2-pyridyl)silyl-2,2-diphenylethene SI-15b

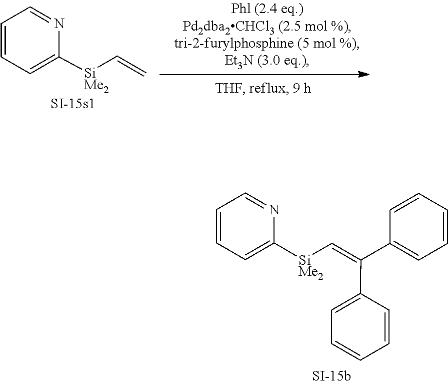

The synthesis was performed following the literature protocol by Yoshida et al. (38)

### Manual Preparation

A solution of 2-pyridyldimethyl(vinyl)silane SI-15s1 (631 mg) in dry THF (total volume 5 ml, 0.773 M), a solution of iodobenzene (1.37 ml) in dry THF (total volume 5 ml, 2.45 M) and a solution of triethylamine (4.22 ml) in THF (total volume 10 ml, 3.03 M) were prepared. The reactor was charged with Pd₂dba₃CHCl₃ (78.4 mg, 0.075 mmol, 0.025 eq.), tri-2-furylphosphine (35.2 mg, 0.15 mmol, 0.05 eq.) And 3 Å molecular sieves (100 mg).

### ChemPU Steps

1) Evacuate reactor and refill with inert gas 3 times, using a vacuum pressure of 10 mbar, waiting 60 s after evacuating and 60 s after refilling with inert gas.
2) Add iodobenzene solution in THF (3.0 ml, 2.45 M, 7.20 mmol, 2.4 eq.) directly to reactor at default speed with stirring at 250 rpm.
3) Add 2-pyridyldimethyl(vinyl)silane SI-15s1 solution in THF (4.0 ml, 0.773 M, 3.10 mmol, 1.0 eq.) directly to reactor at default speed with stirring at 250 rpm.
4) Add triethylamine solution in THF (3.0 ml, 3.03 M, 9.10 mmol, 3.0 eq.) directly to reactor at default speed with stirring at 250 rpm.
5) Heat/Chill reactor to 67° C. For 9 h with stirring at 250 rpm.
6) Heat/Chill reactor to 25° C. With stirring at 250 rpm. Temperature control is stopped once the temperature has been reached.
7) Transfer all from reactor through Silica to rotavap at default speed, without flushing tubing after the transfer.
8) Repeat 3 times:
   a) Add ethyl acetate (60 ml) directly to reactor at default speed with stirring at 250 rpm.
   b) Stir reactor for 60 s at 250 rpm stopping stirring afterwards.
   c) Transfer all from reactor through Silica to rotavap at default speed, without flushing tubing after the transfer.
9) Evaporate contents of rotavap with default pressure control at temperature 50° C. For 30 min.
10) Dry contents of rotavap for 60 min at pressure 10 mbar at temperature 50° C. Stopping heating when step finishes.
11) Shut down the platform.

The crude product was purified by flash column chromatography (50 g Biotage ‘Sfär’ silica column, 60 μm particle size, 100 Å pore size, spherical particle shape) using a gradient of ethyl acetate in petroleum ether (0%-30% over 25 column volumes).

The product 1-dimethyl(2-pyridyl)silyl-2,2-diphenylethene SI-15b was obtained as a pale yellow oil (741 mg, 2.23 mmol, 72%). The recorded NMR signals are in agreement with the literature data. (38)

### 4′-Methoxy-biphenyl-4-carbaldehyde SI-16

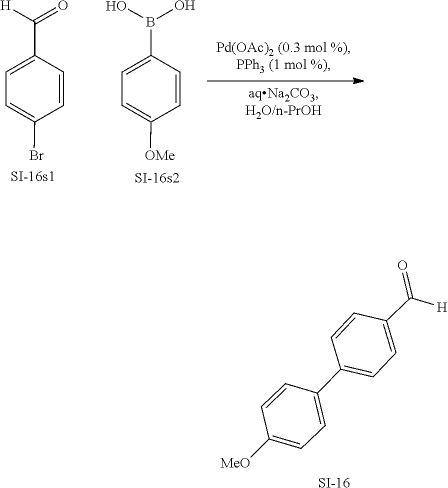

The synthesis was performed following literature protocol by Christopher S. Callam and Todd L. Lowary. (39)

### Manual Preparation

The system was configured as specified in the graph file for this reaction. The Filter reactor was charged with 4-bromobenzyladehyde SI-16s1 (1.00 g, 5.02 mmol, 1.00 Eq.), 4-methoxyphenylboronic acid SI-16s2 (0.692 g, 5.68 mmol, 1.10 eq.), Triphenylphosphine (12.8 mg, 48.8 μmol, 1 mol %) and palladium acetate (3.6 mg, 16.0 μmol, 0.3 mol %).

### ChemPU Steps

1) Confirm aryl halide SI-16s1 (1.00 g, 5.02 mmol, 1.00 eq.) Is in reactor.
2) Confirm aryl boronic acid SI-16s2 (0.692 g, 5.68 mmol, 1.1 eq.) Is in reactor.
3) Add n-propanol (10 ml) directly to reactor at default speed without stirring.
4) Stir reactor for 15 min at 250 RPM stopping stirring afterwards.
5) Confirm palladium acetate (3.6 mg, 16.0 μmol, 0.3 mol %) is in reactor.
6) Confirm triphenylphosphine (12.8 mg, 48.8 μmol, 1 mol %) is in reactor.
7) Add 2M aqueous sodium carbonate (3.25 ml) directly to reactor at default speed with Stirring at 250 RPM.
8) Add deionized water (2 ml) directly to reactor at default speed with stirring at 250 RPM.
9) Heat/Chill reactor to 60° C. For 60 min with stirring at 250 RPM.
10) Heat/Chill reactor to 20° C. With stirring at 250 RPM. Temperature control is Continued after the temperature has been reached.
11) Add water (7 ml) directly to reactor at default speed with stirring at 250 RPM.
12) Stir reactor for 5 min at 250 RPM stopping stirring afterwards.
13) Add ethyl acetate (10 ml) directly to reactor at default speed with stirring at 250 RPM.
14) Extract two-phase mixture in reactor without adding solvent. Transfer waste phase (bottom) to waste, and product phase (top) directly to buffer flask.
15) Transfer all from buffer flask directly to separator at default speed, without flushing Tubing after the transfer.
16) Wash contents of separator with 5% sodium carbonate solution (2×10 ml). Transfer waste phase (bottom) to waste, and product phase (top) directly to separator.
17) Wash contents of separator with brine (2×10 ml). Transfer waste phase (bottom) to waste, and product phase (top) directly to rotavap.
18) Evaporate contents of rotavap with pressure 42 mbar at temperature 50° C. For 30 min.
19) Shut down the platform.

The product 4′-methoxy-biphenyl-4-carbaldehyde: (0.64 g, 3.01 mmol, 61%) was obtained as a white solid. The recorded NMR signals are in agreement with the literature data. (40)

### 2-Methyl-4-(4-nitrophenyl)but-3-yn-2-ol SI-17

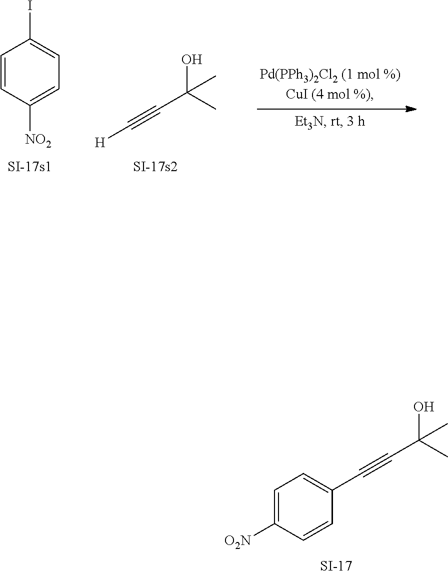

The synthesis was performed following literature protocol by Philippa B. Cranwell, Alexander M. Peterson, Benjamin T. R. Littlefield, and Andrew T. Russell. (41)

### Manual Preparation

The system was configured as specified in the graph file for this reaction. The filter reactor was charged with 1-iodo-4-nitrobenzene SI-17s1 (1.0 g, 4.0 mmol, 1.3 eq.), CuI (37 mg, 0.16 mmol, 4 mol %) and Pd(PPh₃)₂Cl₂ (34 mg, 0.04 mmol, 1 mol %). The solution of 2-methyl-3-butyn-2-ol (0.63M) in Et₃N was prepared manually.

### ChemPU Steps

1) Add 2-methyl-3-butyn-2-ol SI-17s2 in Et₃N (10 ml, 0.63 M, 6.3 mmol, 1.0 eq.) directly to reactor at default speed without stirring.
2) Confirm aryl iodide SI-17s1 (1.24 g, 5 mmol, 1.00 eq.) Is in reactor.
3) Confirm cui (37 mg, 0.20 mmol) is in reactor.
4) Confirm Pd(PPh₃)₂Cl₂ (34 mg, 0.05 mmol) is in reactor.
5) Stir reactor for 3 h at 250 RPM stopping stirring afterwards.
6) Add 2M HCl (50 ml) directly to reactor at default speed with stirring at 250 RPM.
7) Add EtOAc (15 ml) directly to reactor at default speed with stirring at 250 RPM.
8) Extract two-phase mixture in reactor without adding solvent. Transfer waste phase (bottom) to waste, and product phase (top) directly to separator flask.
9) Wash contents of separator flask with 2M HCl (1×20 ml). Transfer waste phase (bottom) to waste, and product phase (top) directly to separator flask.
10) Wash contents of separator flask with aq. Sodium thiosulfate (1×20 ml). Transfer Waste phase (bottom) to waste, and product phase (top) directly to separator flask.
11) Wash contents of separator flask with water (1×20 ml). Transfer waste phase (bottom) to waste, and product phase (top) directly to separator flask.
12) Wash contents of separator flask with brine (1×20 ml). Transfer waste phase (bottom) to waste, and product phase (top) through cartridge to rotavap.
13) Evaporate contents of rotavap with pressure 100 mbar at temperature 40° C. For 60 Min.
14) Shut down the platform.

The product 2-methyl-4-(4-nitrophenyl)but-3-yn-2-ol SI-17 (0.74 g, 3.4 mmol, 84%) was obtained as a brown oil. The recorded NMR signals are in agreement with the literature data. (42)

### 9-Oxabicyclo[6.1.0]nonane SI-18

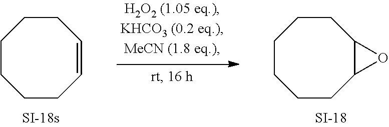

The synthesis was performed following the literature protocol by Bach and Knight. (43)

### Manual Preparations

The system was configured as specified in the graph file for this reaction. The reactor was charged with solid KHCO₃ (885 mg, 8.75 mmol, 0.2 eq.).

### ChemPU Steps

1) Add cis-cyclooctene SI-18s (6.86 ml, 50.0 mmol, 1.00 eq.) directly to reactor at default speed without stirring.
2) Add methanol (34 ml) directly to reactor at default speed with stirring at 250 rpm.
3) Add acetonitrile (4.77 ml, 90.2 mmol, 1.80 eq.) directly to reactor at default speed with stirring at 250 rpm.
4) Heat/Chill reactor to 30° C. With stirring at 250 rpm (continuing temperature control after the temperature has been reached).
5) Add 28% hydrogen peroxide (5.75 ml, 52.3 mmol, 1.05 eq.) directly to reactor over 5 min with stirring at 250 rpm.
6) Add methanol (0.5 ml) directly to reactor at default speed with stirring at 250 rpm.
7) Heat/Chill reactor to 25° C. For 16 h with stirring at 250 rpm.
8) Add brine (57 ml) directly to reactor at default speed with stirring at 250 rpm.
9) Transfer contents of reactor to separate and extract with DCM (4×57 ml). Transfer waste phase (top) to waste and product phase (bottom) to separator.
10) Add aqueous sodium metabisulphite (28 ml) directly to separator at default speed with stirring at 250 rpm.
11) Stir separator for 60 min at 250 rpm. Stop stirring afterwards.
12) Separate phases. Transfer waste phase (top) to waste and product phase (bottom) to rotavap through magnesium sulfate.
13) Evaporate contents of rotavap at 278 mbar at 50° C. For 30 min.
14) Dry contents of rotavap for 30 min at 30 mbar at 50° C. Stop heating afterwards.

The product 9-oxabicyclo[6.1.0]nonane SI-18 (4.66 g, 33.2 mmol, 66%) was Obtained as a white solid. The recorded NMR signals are in agreement with the Literature data. (44)

### Cyclopropane 1,1-dicarboxylic Acid SI-19

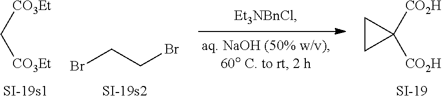

The synthesis was performed following the literature protocol by R. K. Singh and S. Danishefsky. (45)

### Manual Preparations

The reactor was charged with the phase transfer catalyst Triethylbenzylammonium chloride (2.88 g, 13.0 mmol, 0.5 eq.). A stock solution of diethyl Malonate SI-19s1 (26.9 ml, 175 mmol) in 1,2-dibromoethane SI-19s2 (23.1 ml, 263 Mmol) was prepared.

### ChemPU Steps

1) Fill bottom of filter with water (14 ml).
2) Add 50% aqueous NaOH solution (50 ml) directly to filter at default speed with stirring at 600 rpm.
3) Heat/Chill filter to 60° C. With stirring at 250 rpm. Temperature control is continued after the temperature has been reached.
4) Add a solution of diethyl malonate SI-19s1 (3.83 ml, 25.0 mmol, 1.0 eq.) In 1,2-dibromoethane SI-19s2 (3.30 ml, 37.5 mmol, 1.5 eq.) (total volume 7.13 ml) directly to filter at default speed with stirring at 600 rpm.
5) Heat/Chill filter to 65° C. For 10 min with stirring at 600 rpm.
6) Heat/Chill filter to 40° C. For 20 min with stirring at 600 rpm.
7) Heat/Chill filter to 30° C. For 40 min with stirring at 600 rpm.
8) Heat/Chill filter to 25° C. For 60 min with stirring at 600 rpm.
9) Heat/Chill filter to 15° C. With stirring at 250 rpm. Temperature control is continued after the temperature has been reached.
10) Add concentrated hydrochloric acid (65 ml) directly to filter at default speed with stirring at 250 rpm.
11) Heat/Chill filter to 25° C. With stirring at 250 rpm. Temperature control is continued after the temperature has been reached.
12) Add brine (80 ml) directly to filter at default speed with stirring at 250 rpm. 13) Transfer 500 ml from filter directly to separator at default speed, without flushing tubing after the transfer.
14) Fill bottom of filter with water (14 ml).
15) Add water (15 ml) directly to filter at default speed with stirring at 250 rpm.
16) Heat/Chill filter to 25° C. For 10 min with stirring at 600 rpm.
17) Transfer 50 ml from filter directly to separator at default speed, without flushing tubing after the transfer.
18) Extract contents of separator with diethyl ether (8×30 ml). Transfer waste phase (bottom) to waste, and product phase (top) directly to separator.
19) Wash contents of separator with brine (1×20 ml). Transfer waste phase (bottom) to waste, and product phase (top) through anhydrous magnesium sulfate to rotavap.
20) Evaporate contents of rotavap with pressure 440 mbar at temperature 50° C. For 30 min.
21) Dry contents of rotavap for 10 min at default pressure at temperature 50° C. Stopping Heating when step finishes.
22) Add toluene (10 ml) directly to rotavap at default speed without stirring.
23) Stir rotavap for 5 min at 250 rpm stopping stirring afterwards.
24) Transfer 50 ml from rotavap directly to waste at default speed, without flushing tubing after the transfer.
25) Evaporate contents of rotavap with pressure 48 mbar at temperature 50° C. For 10 min.
26) Dry contents of rotavap for 30 min at default pressure at temperature 50° C. Stopping heating when step finishes.
27) Shut down the platform.

The product cyclopropane 1,1-dicarboxylic acid SI-19 (2.02 g, 15.5 mmol, 62%) was obtained as a white solid. The recorded NMR signals are in agreement with the literature data. (46)

### 2-(1H-Indol-3-yl)ethan-1-ol SI-20

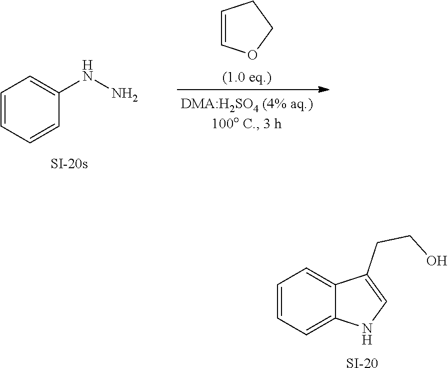

The synthesis was performed following the literature protocol by Tillye et al. (47)

### Manual Preparations

The system was configured as specified in the graph file for this reaction.

### ChemPU Steps

1) Add phenylhydrazine (2.95 ml, 30 mmol, 1.0 eq.) DIRECTLY to reactor at default speed without stirring.
2) Add DMA (40 ml) directly to reactor at default speed without stirring.
3) Add 4% H₂SO₄ (40 ml) directly to reactor at default speed with stirring at 250 RPM.
4) Heat/Chill reactor to 100° C. With stirring at 250 RPM. Temperature control is continued after the temperature has been reached.
5) Add 2,3-dihydrofuran (2.27 ml, 30 mmol, 1.0 eq.) directly to reactor over 1 hour with stirring at 250 RPM.
6) Heat/Chill reactor to 100° C. For 3 h with stirring at 250 RPM.
7) Allow to warm/cool reactor to 30° C. With stirring at 250 RPM. Temperature control is stopped once the temperature has been reached.
8) Transfer all from reactor directly to separator at default speed, without flushing tubing after the transfer.
9) Repeat 2 times:
   a) Add EtOAc (20 ml) directly to reactor at default speed with stirring at 250 RPM.
   b) Stop stirring reactor.
   c) Transfer all from reactor directly to separator at default speed, without flushing tubing after the transfer.
10) Extract contents of separator with EtOAc (3×40 ml). Transfer waste phase (bottom) to waste, and product phase (top) directly to separator.
11) Wash contents of separator with NaCl (5×50 ml). Transfer waste phase (bottom) to waste, and product phase (top) directly to separator.
12) Transfer all from separator through MgSO₄ to rotavap at default speed, without flushing tubing after the transfer. 13) Transfer 30 ml from separator through MgSO₄ to rotavap at default speed, without flushing tubing after the transfer.
14) Evaporate contents of rotavap with pressure 30 mbar at temperature 40° C. For 60 min.

The crude product was obtained as a viscous oil, containing the desired product (ca. 60-65% yield by NMR analysis) and DMA. If the crude product contains a significant amount of DMA, the residue can be washed with 0.5M HCl to remove DMA. The residue was purified by column chromatography on alumina (5% to 50% EtOAc/pet. Ether) to give a dark orange oil.

The oil was dissolved in minimal amount of CH₂Cl₂ and triturated from hexane.

The product 2-(1H-indol-3-yl)ethan-1-ol SI-20 (1.59 g, 9.86 mmol, 33%) was obtained as a pale orange solid. The recorded NMR signals are in agreement with the literature data. (47)

### 2,6-Di-tert-butyl-4-methylpyrylium trifluoromethanesulfonate SI-21

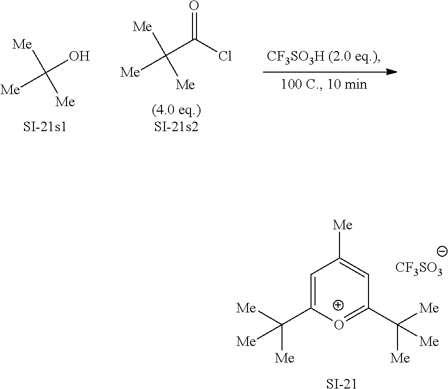

The synthesis was performed following the literature protocol by A. G. Anderson and P. J. Stang. (48)

### Manual Preparation

Tert-butanol SI-21s1 was heated to 50° C. Shortly before use.

### ChemPU Steps

1) Dry contents of filter for 30 min at default pressure at temperature 120° C. Stopping heating when step finishes.
2) Fill bottom of filter with anhydrous tert-butyl alcohol (14 ml).
3) Add pivaloyl chloride SI-21s2 (20.3 ml, 161 mmol, 4.0 eq.) directly to filter at default speed with stirring at 250 rpm.
4) Add anhydrous tert-butyl alcohol SI-21s1 (3.86 ml, 40.0 mmol, 1.0 eq.) directly to filter at default speed with stirring at 250 rpm.
5) Heat/Chill filter to 85° C. With stirring at 250 rpm. Temperature control is continued after the temperature has been reached.
6) Add trifluoromethanesulfonic acid (7.21 ml, 80.6 mmol, 2.0 eq.) directly to filter over 3 min with stirring at 250 rpm.
7) Heat/Chill filter to 100° C. for 10 min with stirring at 250 rpm.
8) Heat/Chill filter to −10° C. with stirring at 250 rpm. Temperature control is continued after the temperature has been reached.
9) Add diethyl ether (65 mL) directly to filter at default speed with stirring at 250 rpm.
10) Transfer 500 mL from filter directly to waste at default speed, without flushing tubing after the transfer.
11) Dry contents of filter for 2 min at default pressure without temperature control stopping heating when step finishes.
12) Repeat 3 times:
   a) Add diethyl ether (20 mL) directly to filter at default speed with stirring at 250 rpm.
   b) Heat/Chill filter to −10° C. for 5 min with stirring at 600 rpm.
   c) Transfer 100 mL from filter directly to waste at default speed, without flushing tubing after the transfer.
   d) Dry contents of filter for 2 min at default pressure without temperature control stopping heating when step finishes.
13) Dry contents of filter for 60 min at default pressure without temperature control stopping heating when step finishes.
14) Shut down the platform.

The product 2,6-di-tert-butyl-4-methylpyrylium trifluoromethanesulfonate SI-21 (5.07 g, 14.2 mmol, 36%) was obtained as a white solid. The recorded NMR signals are in agreement with the literature data. (49)

### Ethyl 5-hydroxy-1,2-dimethyl-1H-indole-3-carboxylate SI-22

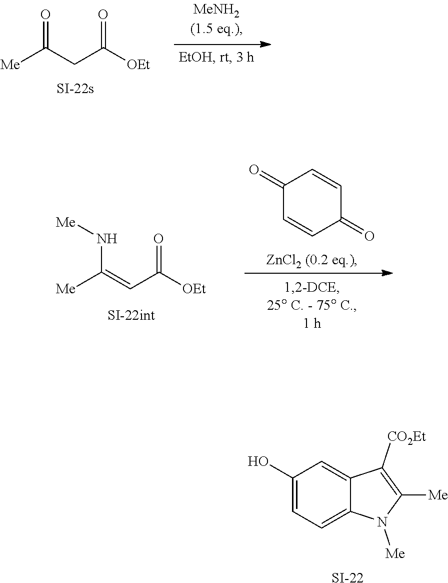 [SMILES: CCOC(=O)CC(C)=O ; CCOC(=O)/C=C(/C)NC ; O=C1C=CC(=O)C=C1 ; CCOC(=O)c1c(C)n(C)c2ccc(O)cc12 ; "SI-22"]

The synthesis was performed according to the modified literature protocol by Brennan et al. (50)

### Manual Preparation

p-Benzoquinone (90 g) was recrystallized from boiling ethanol/water (500 mL, 95/5 v/v) and dried under high vacuum over night before use. A 1.6 M solution of p-benzoquinone (4.34 g, 40.1 mmol) in DCE (25 mL) was prepared.

ZnCl₂ (905 mg, 6.61 mmol, 0.20 eq.) was added manually in the corresponding step.

### ChemPU Steps

1) Add ethyl acetoacetate SI-22s (4.47 mL, 35.0 mmol, 1.06 eq.) directly to reactor_1 at default speed without stirring.
2) Add N-methylamine in EtOH (6.47 mL, 52.0 mmol, 1.5 eq., 33% v/v) directly to reactor_1 over 20 min with stirring at 250 RPM.
3) Stir reactor_1 for 3 h at 250 RPM stopping stirring afterwards.
4) Transfer all from reactor_1 directly to rotavap at default speed, without flushing tubing after the transfer.
5) Evaporate contents of rotavap with pressure 100 mbar at temperature 50° C. for 30 min.
6) Evaporate contents of rotavap with pressure 50 mbar at temperature 50° C. for 30 min.
7) Dry contents of rotavap for 60 min at pressure 10 mbar at temperature 80° C. stopping heating when step finishes.
8) Add ZnCl₂ (0.905 g, 6.61 mmol, 0.20 eq.) to reactor_2.
9) Heat/Chill reactor_2 to 55° C. with stirring at 250 RPM. Temperature control is continued after the temperature has been reached.
10) Add p-benzoquinone in DCE (21 mL, 1.57 M, 33.0 mmol, 1.0 equiv) directly to reactor_2 at default speed without stirring.
11) Transfer imine intermediate SI-22int from rotavap directly to reactor_2 at default speed, without flushing tubing after the transfer.
12) Heat/Chill reactor_2 to 55° C. for 60 min with stirring at 250 RPM.
13) Heat/Chill reactor 2 to 15° C. for 5 min with stirring at 250 RPM.
14) Filter contents of reactor_2, applying vacuum for 30 min, with stirring at 100 RPM, discarding filtrate using standard transfer speeds.
15) Dry contents of reactor_2 for 30 min at default pressure at temperature 15° C. stopping heating when step finishes.
16) Heat/Chill reactor_2 to 15° C. with stirring at 250 RPM. Temperature control is continued after the temperature has been reached.
17) Repeat 1 times:
   a) Add ethanol (3.0 mL) directly to reactor_2 at default speed without stirring.
   b) Add water (15 mL) directly to reactor_2 at default speed without stirring.
   c) Heat/Chill reactor_2 to 15° C. for 60 min with stirring at 500 RPM.
   d) Filter contents of reactor 2, applying vacuum for 30 min, with stirring at 100 RPM, discarding filtrate using standard transfer speeds.
18) Dry contents of reactor_2 for 60 min at default pressure at temperature 50° C. stopping heating when step finishes.

In order to analyse the imine intermediate SI-22int and determine the yield, the procedure was briefly interrupted. The intermediate ethyl (Z)-3-(methylamino)but-2-enoate SI-22int was obtained as a pale yellow oil (4.73 g, 33.0 mmol, 94%). The recorded NMR signals are in agreement with the literature data. (51)

The product ethyl 5-hydroxy-1,2-dimethyl-1H-indole-3-carboxylate SI-22 was obtained as a beige solid (4.67 g, 19.6 mmol, 59%). The recorded NMR signals are in agreement with the literature data. (50)

### 3-Benzoyl-1-Vinylpyrrolidin-2-One—SI-27

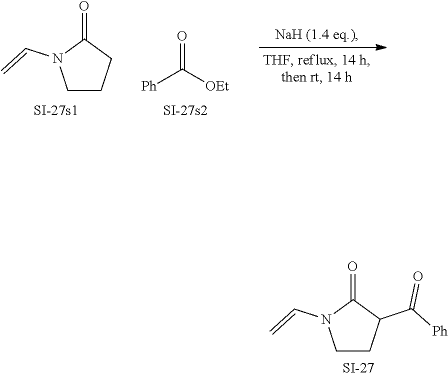

The synthesis was performed following the literature protocol by Sorgi, et al. (52)

### Manual Preparations

The system was configured as specified in the graph file.

Sodium hydride (2.50 g, 62.5 mmol, 1.4 eq.) was added manually in the corresponding step.

In a 20 mL vial was weighed N-vinylpyrrolidin-2-one (8 g, 72 mmol) and ethyl benzoate (10.9 g, 72.7 mmol) and mixed well. Note: the volume required to be injected from this mixture was calculated beforehand by mixing N-vinylpyrrolidin-2-one (5 g, 45 mmol, 1 eq.) and ethyl benzoate (6.8 g, 45.4 mmol, 1.01 eq.) and their volume was determined to be 11.2 mL.

Note: It is important to adjust the flow of the argon (inert gas) that is used for purging the reaction in the jacketed filter to avoid any excessive evaporation of the solvent.

### ChemPU Steps

1) CSwitchArgon
2) Add sodium hydride (2.50 g, 62.5 mmol, 1.4 eq.) to jacketed_filter.
3) Add anhydrous_THF (40 mL) directly to jacketed_filter at default speed with stirring at 250 RPM.
4) Async
   a) Purge jacketed_filter with inert gas for 15 h.
5) Async
   a) Heat/Chill jacketed_filter to 66° C. for 14 h with stirring at 500 RPM.
6) Add ethyl_benzoate_N-vinylpyrrolidin-2-one_mixture (11.2 mL) directly to jacketed_filter over 4 h with stirring at 500 RPM.
7) Wait for 10 h.
8) Heat/Chill jacketed_filter to 25° C. for 14 h with stirring at 500 RPM. Temperature control is continued after the temperature has been reached.
9) Add saturated_aqueous_ammonium_chloride (30 mL) directly to jacketed_filter over 10 min with stirring at 500 RPM.
10) Stir jacketed_filter for 5 min at 500 RPM stopping stirring afterwards.
11) Transfer 200 mL from jacketed_filter directly to separator at default speed without flushing tubing after the transfer.
12) Repeat the following 2 times.
   a) Add toluene (50 mL) directly to jacketed_filter at default speed with stirring at 250 RPM.
   b) Add saturated_aqueous_ammonium_chloride (30 mL) directly to jacketed_filter at default speed with stirring at 250 RPM.
   c) Stir jacketed_filter for 5 min at 500 RPM stopping stirring afterwards.
   d) Transfer 150 mL from jacketed filter directly to separator at default speed without flushing tubing after the transfer.
13) Wash contents of separator with saturated_aqueous_ammonium_chloride (2×10 mL). Transfer waste phase (bottom) to vessel separator, and product phase (top) through MgSO4_sand to rotavap.
14) Extract contents of separator with toluene (2×40 mL). Transfer waste phase (bottom) to vessel buffer_flask_1, and product phase (top) through MgSO4_sand to rotavap.
15) Evaporate contents of rotavap with pressure 1 mbar at temperature 30° C. for 30 min.

The crude product was purified by recrystallization from iPrOH.

The product 3-benzoyl-1-vinylpyrrolidin-2-one SI-24 was obtained as off-white crystals (5.2 g, 23.7 mmol, 52%). The recorded ¹H-NMR signals agree with the literature data. (52)

### 5-Phenyl-3,4-dihydro-2H-pyrrole SI-28

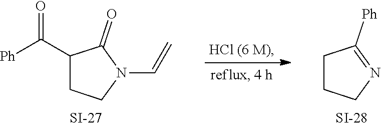

The synthesis was performed following the literature protocol by Maryanoff et al. (53)

### Manual Preparations

The system was configured as specified in the graph file for this reaction. The corresponding reagent flask‘SM’ was charged with a weighed amount of 3-benzoyl-N-vinylpyrrolidin-2-one (2.15 g, 10.0 mmol, 1.0 eq.).

### ChemPU Steps

1) Add 6M HCl (10 mL) directly to reactor at default speed without stirring.
2) Heat/Chill reactor to 100° C. with stirring at 250 RPM. Temperature control is continued after the temperature has been reached.
3) Add tetrahydrofuran (2 mL) directly to SM at default speed without stirring.
4) Transfer 7 mL from SM directly to reactor at default speed, without flushing tubing after the transfer.
5) Transfer 1 mL from SM directly to reactor at default speed, flushing tubing after the transfer.
6) Heat/Chill reactor to 100° C. for 4 h with stirring at 250 RPM.
7) Allow to warm/cool reactor to 25° C. with stirring at 250 RPM. Temperature control is stopped once the temperature has been reached.
8) Add conc. NaOH (6 mL) directly to reactor at default speed with stirring at 250 RPM.
9) Transfer all from reactor directly to separator at default speed, without flushing tubing after the transfer.
10) Repeat 2 times:
   a) Add water (20 mL) directly to reactor at default speed without stirring.
   b) Add CH₂Cl₂ (20 mL) directly to reactor at default speed without stirring.
   c) Transfer all from reactor directly to separator at default speed, without flushing tubing after the transfer.
11) Extract contents of separator with CH₂Cl₂ (3×30 mL). Transfer waste phase (top) to waste, and product phase (bottom) through MgSO₄ to rotavap.
12) Transfer 30 mL from separator through MgSO₄ to rotavap at default speed, without flushing tubing after the transfer.
13) Evaporate contents of rotavap with pressure 150 mbar at temperature 40° C. for 45 min.

The crude product was purified by column chromatography (0 to 40% EtOAc/pet. ether).

The product 5-phenyl-3,4-dihydro-2H-pyrrole SI-28 (634 mg, 4.37 mmol, 44%) was obtained as a yellow oil. The recorded NMR signals are in agreement with the literature data. (53)

### 5,11,17,23,29,35,41,47-octakis(tert-butyl)-49,50,51,52,53,54,55,56-octakis(hydroxy)calix[8]arene SI-29

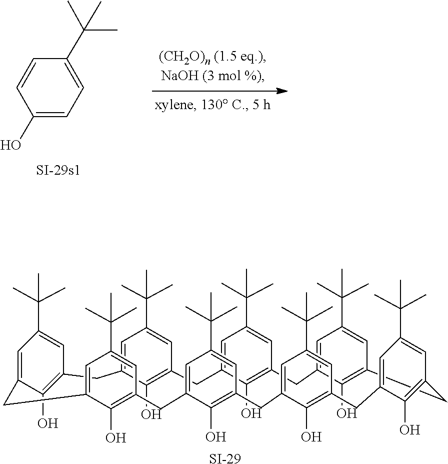

The synthesis was performed following the literature protocol by Munch and Gutsche. (54)

### Manual Preparations

The system was configured as specified in the graph file for this reaction. The reactor was charged with solid p-tert-butylphenol SI-29s1 (5.0 g, 33 mmol, 1.0 eq.) and paraformaldehyde (1.5 g, 50 mmol, 1.5 eq.). Sodium hydroxide (40 mg, 0.1 mmol, 0.03 eq.) was added manually in the corresponding step.

### ChemPU Steps

1) Add p-xylene (50 mL) directly to reactor at default speed with stirring at 250 rpm.
2) Add sodium hydroxide (40 mg, 0.1 mmol, 0.03 eq.) directly to reactor.
3) Heat/Chill reactor to 150° C. for 5 h with stirring at 450 rpm.
4) Heat/Chill reactor to 30° C. with stirring at 150 rpm (continuing temperature control after the temperature has been reached).
5) Wait for 15 min.
6) Transfer 80 mL from reactor directly to filter at default speed, flushing tubing with xylene after the transfer.
7) Transfer 100 mL from filter directly to buffer_flask2 at default speed, without flushing tubing after the transfer.
8) Dry contents of filter for 5 min at default pressure without temperature control stopping heating when step finishes.
9) Fill bottom of filter with nitrogen gas (10 mL).
10) Add 40 mL of chloroform directly filter with stirring at 150 rpm.
11) Heat/Chill filter to 60° C. for 50 min with stirring at 100 rpm.
12) Heat/Chill filter to 10° C. without stirring (continuing temperature control after the temperature has been reached).
13) Wait for 30 min.
14) Transfer 80 mL from filter directly to buffer_flask1 at default speed, without flushing tubing after the transfer.
15) Dry contents of filter for 10 min at default pressure without temperature control stopping heating when step finishes.

The product 5,11,17,23,29,35,41,47-octakis(tert-butyl)-49,50,51,52,53,54,55,56-octakis(hydroxy)calix[8]arene SI-29 (1.94 g, 1.49 mmol, 36%) was obtained as a white solid. The recorded NMR signals are in agreement with the literature data. (55)

### 49,50,51,52,53,54,55,56-octakis(hydroxy)calix[8]arene SI-30

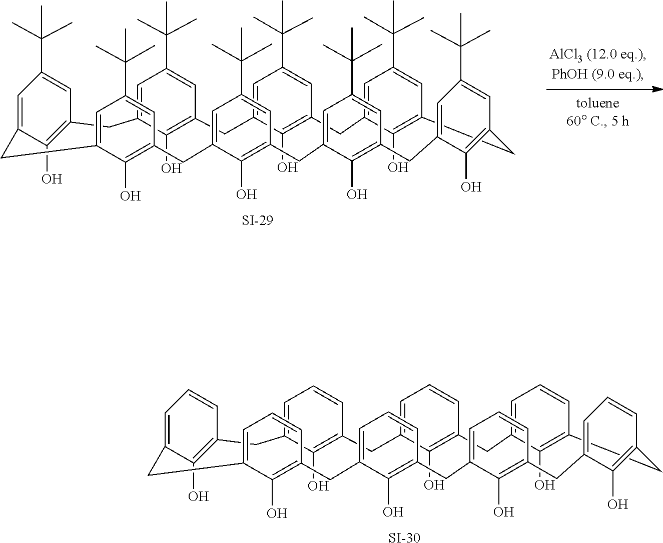

The synthesis was performed following the literature protocol by Zhang et al. (56)

### Manual Preparations

The system was configured as specified in the graph file for this reaction. Toluene was dried over 3 Å molecular sieves for 24 h prior to using. The reactor was charged with solid calix[8]arene SI-29 (1.0 g, 0.77 mmol, 1 eq.) and phenol (0.65 g, 6.9 mmol, 9.0 eq.).

AlCl₃ (1.23 g, 6.90 mmol, 12 eq.) was added manually in the corresponding step.

### ChemPU Steps

1) Add toluene (40 mL) directly to reactor at default speed with stirring at 220 rpm.
2) Stir reactor for 5 min at 320 rpm stopping stirring afterwards.
3) Add AlCl₃ (1.23 g, 6.90 mmol, 12.0 eq.) directly to reactor at default speed.
4) Heat/Chill reactor to 60° C. for 5 h with stirring at 450 rpm.
5) Heat/Chill reactor to 30° C. with stirring at 450 rpm (continuing temperature control after the temperature has been reached).
6) Add HCl (35 mL) directly to reactor at default speed with stirring at 420 rpm.
7) Stir reactor for 45 min at 500 RPM stopping stirring afterwards.
8) Transfer 90 mL from reactor directly to rotavap at default speed, flushing tubing with toluene after the transfer.
9) Evaporate contents of rotavap with pressure 63 mbar at temperature 55° C. for 30 min.
10) Add methanol (50 mL) directly to rotavap at default speed without stirring.
11) Evaporate contents of rotavap with pressure 1000 mbar at temperature 66° C. for 60 min.
12) Fill bottom of filter with nitrogen gas (10 mL).
13) Transfer 80 mL from rotavap directly to filter at default speed, flushing tubing with methanol after the transfer.
14) Transfer 100 mL from filter directly to buffer_flask at default speed, without flushing tubing after the transfer.
15) Dry contents of filter for 10 min at default pressure without temperature control stopping heating when step finishes.

The product 49,50,51,52,53,54,55,56-octakis(hydroxy)calix[8]arene SI-30 (0.50 g, 0.59 mmol, 77%) was obtained as a light beige solid. The recorded NMR signals are in agreement with the literature data. (56)

### (R,R)-1,2-Diammoniumcyclohexane mono-(+)-tartrate SI-31

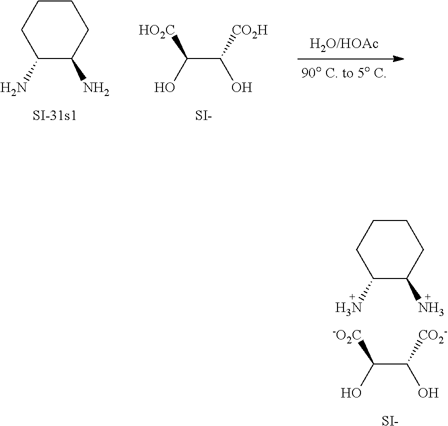

The synthesis was performed following the literature protocol by Larrow and Jacobsen. (57)

### Manual Preparation

The system was configured as specified in the graph file for this reaction. L-(+)-Tartaric acid (15.0 g, 100 mmol, 1.00 eq.) was manually added to the filter flask before the start of this procedure.

### ChemPU Steps

1) Confirm L-(+)-Tartaric acid (15.0 g, 100 mmol, 1.00 eq.) is in filter.
2) Fill bottom of filter with water (10 mL).
3) Dissolve contents of filter in water (37.5 mL) at 25° C. over 10 min, stirring at 400 RPM.
4) Add racemic trans-1,2-diaminocyclohexane (24.0 mL, 200 mmol, 2.00 eq.) directly to filter at default speed with stirring at 250 RPM.
5) Allow to warm/cool filter to 90° C. with stirring at 250 RPM. Temperature control is continued after the temperature has been reached.
6) Add glacial acetic acid (7.5 mL) directly to filter at default speed with stirring at 250 RPM.
7) Allow to warm/cool filter to 80° C. with stirring at 250 RPM. Temperature control is continued after the temperature has been reached.
8) Allow to warm/cool filter to 60° C. with stirring at 250 RPM. Temperature control is continued after the temperature has been reached.
9) Allow to warm/cool filter to 40° C. with stirring at 250 RPM. Temperature control is continued after the temperature has been reached.
10) Allow to warm/cool filter to 20° C. with stirring at 250 RPM. Temperature control is continued after the temperature has been reached.
11) Allow to warm/cool filter to 5° C. with stirring at 250 RPM. Temperature control is continued after the temperature has been reached.
12) Stir filter for 60 min at 250 RPM leaving stirring on afterwards.
13) Remove dead volume (10 mL) from bottom of filter.
14) Filter contents of filter, applying vacuum for 2 min, with stirring at 500 RPM, discarding filtrate using standard transfer speeds.
15) Wash solid in filter with water (1×7.5 mL) at 5° C., with stirring at 500 RPM, applying vacuum for 5 min. discarding filtrate.
16) Wash solid in filter with methanol (4×7.5 mL) at 5° C., with stirring at 500 RPM, applying vacuum for 5 min, discarding filtrate.
17) Dry contents of filter for 60 min at default pressure at temperature 42.5° C. stopping heating when step finishes.
18) Shut down the platform.

The product (R,R)-1,2-Diammoniumcyclohexane mono-(+)-tartrate (20.0 g, 75.8 mmol, 76%) was obtained as a white solid. The recorded NMR signals are in agreement with the literature data.

### (R,R)—N,N′-Bis(3,5-di-tert-butylsalicylidene)-1,2-cyclohexanediamine SI-32

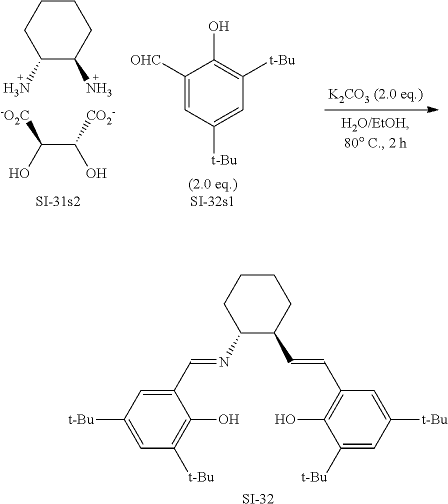

The synthesis was performed following the literature protocol by Larrow and Jacobsen. (57)

### Manual Preparation

The system was configured as specified in the graph file for this reaction. The filter reactor was charged with (R,R)-1,2-diammoniumcyclohexane mono-(+)-tartrate salt SI-31s2 (2.9 g, 11.2 mmol, 1.00 eq.) and potassium carbonate (3.12 g, 22.5 mmol, 2.00 eq.) manually in the corresponding steps.

### ChemPU Steps

1) Add (R,R)-1,2-diammoniumcyclohexane mono-(+)-tartrate salt SI-31s2 (2.90 g, 11.2 mmol, 1.00 eq.) to filter.
2) Add potassium carbonate (3.12 g, 22.5 mmol, 2.00 eq.) to filter.
3) Fill bottom of filter with water (10 mL).
4) Add water (15 mL) directly to filter at default speed without stirring.
5) Stir filter for 20 min at 250 RPM leaving stirring on afterwards.
6) Add ethanol (60 mL) directly to filter at default speed with stirring at 250 RPM.
7) Heat/Chill filter to 78.5° C. with stirring at 250 RPM. Temperature control is continued after the temperature has been reached.
8) Add 3,5-di-tert-butylsalicylaldehyde SI-32s1 ethanol solution (22.9 mL, 1M, 22.9 mmol, 2 eq.) directly to filter over 30 min with stirring at 250 RPM.
9) Add ethanol (5 mL) directly to filter at default speed with stirring at 250 RPM.
10) Stir filter for 2 h at 250 RPM stopping stirring afterwards.
11) Heat/Chill filter to 60° C. with stirring at 250 RPM. Temperature control is continued after the temperature has been reached.
12) Wait for 20 min.
13) Add water (15 mL) directly to filter at default speed without stirring.
14) Heat/Chill filter to 50° C. with stirring at 250 RPM. Temperature control is continued after the temperature has been reached.
15) Wait for 20 min.
16) Heat/Chill filter to 40° C. with stirring at 250 RPM. Temperature control is continued after the temperature has been reached.
17) Wait for 20 min.
18) Heat/Chill filter to 20° C. with stirring at 250 RPM. Temperature control is continued after the temperature has been reached.
19) Wait for 20 min.
20) Heat/Chill filter to 0° C. with stirring at 250 RPM. Temperature control is continued after the temperature has been reached.
21) Stir filter for 60 min at 250 RPM stopping stirring afterwards.
22) Remove dead volume (10 mL) from bottom of filter.
23) Filter contents of filter, applying vacuum for 2 min, with stirring at 500 RPM, discarding filtrate using standard transfer speeds.
24) Wash solid in filter with ethanol (1×10 mL) without temperature control, with stirring at 500 RPM, applying vacuum for 10 s, discarding filtrate.
25) Dry contents of filter for 60 min at default pressure without temperature control stopping heating when step finishes.
26) Fill bottom of filter with methylene chloride (10 mL).
27) Dissolve contents of filter in methylene chloride (50 mL) at 25° C. over 20 min, stirring at 400 RPM.
28) Transfer 100 mL from filter directly to separator at default speed, without flushing tubing after the transfer.
29) Wash contents of separator with water (2×300 mL). Transfer waste phase (top) to waste, and product phase (bottom) directly to separator.
30) Wash contents of separator with saturated aqueous sodium chloride (1×300 mL). Transfer waste phase (top) to waste, and product phase (bottom) through sodium sulfate to rotavap.
31) Evaporate contents of rotavap with default pressure control at temperature 25° C. for 30 min.
32)Shut down the platform.

The product (R,R)—N,N′-Bis(3,5-di-tert-butylsalicylidene)-1,2-cyclohexanediamine SI-32 (5.3 g, 9.5 mmol, 85%) was obtained as a yellow solid. The recorded NMR signals are in agreement with the literature data. (57)

### (R,R)—N,N′-Bis(3,5-di-tert-butylsalicylidene)-1,2-cyclohexanediamino manganese(II) chloride SI-33

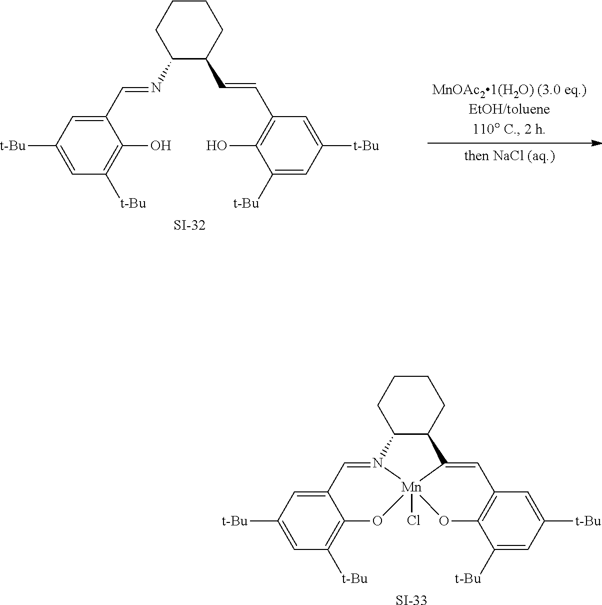

The synthesis was performed following the literature protocol by Larrow and Jacobsen. (57)

### Manual Preparation

The system was configured as specified in the graph file for this reaction. The filter reactor was charged with manganese acetate tetrahydrate (1.12 g, 4.5 mmol, 3.0 eq.)

### ChemPU Steps

1) Dissolve contents of reactor in ethanol (50 mL) at 25° C. over 10 min, stirring at 400 RPM.
2) Heat/Chill reactor to 77.5° C. with stirring at 250 RPM. Temperature control is continued after the temperature has been reached.
3) Add (R,R)—N,N′-bis(3,5-di-tert-butylsalicylidene)-1,2-cyclohexanediamine SI-32 toluene solution (12.5 mL, 0.12 M, 1.5 mmol, 1 eq.) directly to reactor over 45 min without stirring.
4) Add toluene (5 mL) directly to reactor at default speed with stirring at 250 RPM.
5) Heat/Chill reactor to 110.6° C. for 2 h with stirring at 250 RPM.
6) Add saturated aqueous sodium chloride (10 mL) directly to reactor at default speed with stirring at 250 RPM.
7) Heat/Chill reactor to 25° C. with stirring at 250 RPM. Temperature control is continued after the temperature has been reached.
8) Add toluene (30 mL) directly to reactor at default speed with stirring at 250 RPM.
9) Stir reactor for 5 min at 250 RPM stopping stirring afterwards.
10) Transfer 300 mL from reactor directly to separator at default speed, flushing tubing after the transfer.
11) Wash contents of separator with water (3×60 mL). Transfer waste phase (bottom) to waste, and product phase (top) directly to separator.
12) Wash contents of separator with saturated aqueous sodium chloride (1×50 mL). Transfer waste phase (bottom) to waste, and product phase (top) through anhydrous sodium sulfate to rotavap.
13) Evaporate contents of rotavap with pressure 190 mbar at temperature 40° C. for 35 min.
14) Dissolve contents of rotavap in methylene chloride (30 mL) at 25° C. over 20 min, stirring at 280 RPM.
15) Add heptane (30 mL) directly to rotavap at default speed with stirring at 250 RPM.
16) Evaporate contents of rotavap with default pressure control at temperature 40° C. for 35 min.
17)Shut down the platform.

The product, (R,R)—N,N-Bis(3,5-di-tert-butylsalicylidene)-1,2-cyclohexanediamino manganese(III) chloride SI-33 (0.65 g, 1.05 mmol, 70%) was obtained as a dark brown solid. A melting point 2320° C. was observed. The complex does not exhibit a readily interpretable NMR spectrum because of the paramagnetic nature of the complex. (57)

### Cholesta-3,5-dien-3-yl trifluoromethanesulfonate SI-34

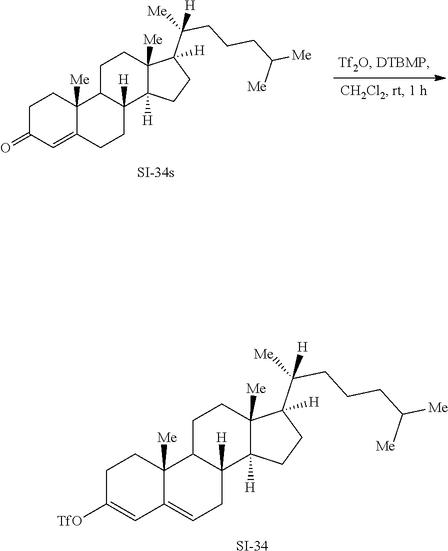

The synthesis was performed following the literature protocol by Ortar et al. (58)

### Manual Preparations

The system was configured as specified in the graph file for this reaction. The following stock solutions were prepared in dry DCM (dried over activated 3 Å molecular sieves for 24 h):
2,6-di-tert-Butyl-4-meththylpyridine (5.25 g) in DCM (45 mL).
Cholestenone (6.83 g) in DCM (45 mL).

### ChemPU Steps

1) Dry contents of reactor for 30 min at default pressure at temperature 120° C. (stop heating when step is finished).
2) Evacuate reactor and refill with inert gas 3 times, using a vacuum pressure of 50 mbar, waiting 60 s after evacuating and 60 s after refilling with inert gas.
3) Heat/Chill reactor to 25° C. for 2 min with stirring at 250 rpm.
4) Add dry dichloromethane solution of base 2,6-di-tert-butyl-4-methylpyridine (40 mL, 0.38 M, 4.67 g, 22.5 mmol, 1.5 eq.) directly to reactor at default speed without stirring.
5) Add trifluoromethanesulfonic anhydride (3.32 mL, 18.8 mmol, 1.3 eq.) directly to reactor at default speed with stirring at 250 rpm.
6) Add dry dichloromethane (3.3 mL) directly to reactor at default speed with stirring at 250 rpm.
7) Add dry dichloromethane solution of cholest-4-en-3-one SI-34s (40 mL, 0.38 M, 6.07 g, 15 mmol, 1.0 eq.) directly to reactor over 17.5 min with stirring at 250 rpm.
8) Add dry dichloromethane (3.3 mL) directly to reactor at default speed with stirring at 250 rpm.
9) Heat/Chill reactor to 25° C. for 60 min with stirring at 250 rpm.
10) Transfer all from reactor directly to rotavap at default speed, without flushing tubing after the transfer.
11) Add dry dichloromethane (50 mL) directly to reactor at default speed with stirring at 250 rpm.
12) Transfer all from reactor directly to rotavap at default speed, without flushing tubing after the transfer.
13) Evaporate contents of rotavap with pressure 699 mbar at temperature 50° C. for 30 min.
14) Add diethyl ether (40 mL) directly to rotavap at default speed with stirring at 250 rpm.
15) Transfer 100 mL from rotavap directly to separator at default speed, without flushing tubing after the transfer.
16) Add diethyl ether (40 mL) directly to rotavap at default speed with stirring at 250 rpm.
17) Transfer 100 mL from rotavap directly to separator at default speed, without flushing tubing after the transfer.
18) Add diethyl ether (40 mL) directly to rotavap at default speed with stirring at 250 rpm.
19) Transfer 100 mL from rotavap directly to separator at default speed, without flushing tubing after the transfer.
20) Add diethyl ether (40 mL) directly to rotavap at default speed with stirring at 250 rpm.
21) Transfer 100 mL from rotavap directly to separator at default speed, without flushing tubing after the transfer.
22) Add methanol (100 mL) directly to rotavap at default speed with stirring at 250 rpm.
23) Transfer 125 mL from rotavap directly to waste at default speed, without flushing tubing after the transfer.
24) Add methanol (100 mL) directly to rotavap at default speed with stirring at 250 rpm.
25) Transfer 125 mL from rotavap directly to waste at default speed, without flushing tubing after the transfer.
26) Add methanol (100 mL) directly to rotavap at default speed with stirring at 250 rpm.
27) Transfer 125 mL from rotavap directly to waste at default speed, without flushing tubing after the transfer.
28) Wash contents of separator with 2 N hydrochloric acid (2×100 mL). Transfer waste phase (bottom) to waste, and product phase (top) directly to separator.
29) Wash contents of separator with brine (3×100 mL). Transfer waste phase (bottom) to waste, and product phase (top) through anhydrous potassium carbonate to rotavap.
30) Evaporate contents of rotavap with pressure 699 mbar at temperature 50° C. for 30 min.
31) Dry contents of rotavap for 30 min at default pressure at temperature 50° C. stopping heating when step finishes.

The product 9-oxabicyclo[6.1.0]nonane SI-34 (7.99 g, 15.0 mmol, >99%) was obtained as a beige solid. The recorded NMR signals are in agreement with the literature data. (58)

### Cholesta-3,5-diene SI-35

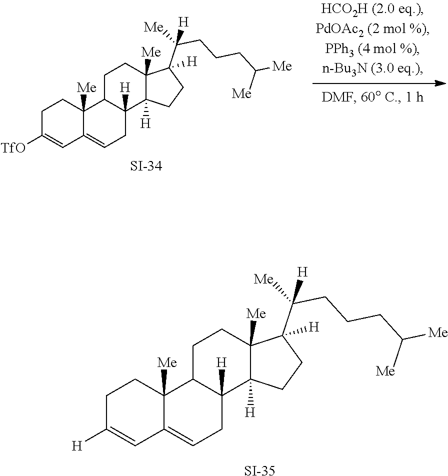

The synthesis was performed following the literature protocol by Ortar et al. (58)

### Manual Preparations

DMF was dried over activated 3 Å molecular sieves for 24 h. A solution of formic acid (1.94 M) in DMF was prepared. The reactor was charged with cholesta-3,5-dien-3-yl trifluoromethanesulfonate SI-34 (1.04 g, 2.00 mmol, 1.0 eq.), Pd(OAc)₂ (8.3 mg, 0.04 mmol, 0.02 eq.) and PPh₃ (20.8 mg, 0.079 mmol, 0.04 eq.).

### ChemPU Steps

1) Evacuate reactor and refill the reactor with inert gas 3 times, using a vacuum pressure of 50 mbar, waiting 60 s after evacuating and 60 s after refilling with inert gas.
2) Add tributylamine (3.61 mL, 15.0 mmol, 3.0 eq.) directly to reactor at default speed without stirring.
3) Add DMF (7.75 mL) directly to reactor at default speed with stirring at 250 rpm.
4) Transfer 400 mL from argon source directly to reactor at default speed, without flushing tubing after the transfer.
5) Add formic acid DMF solution (2.58 mL, 1.94 M, 10.0 mmol, 2.5 eq.) directly to reactor over 2.5 min with stirring at 250 rpm.
6) Heat/Chill reactor to 60° C. for 60 min with stirring at 250 rpm.
7) Add 2 N hydrochloric acid (50 mL) directly to separator at default speed without stirring.
8) Add diethyl ether (25 mL) directly to reactor at default speed with stirring at 250 rpm.
9) Transfer 50 mL from reactor directly to separator at default speed, without flushing tubing after the transfer.
10) Add diethyl ether (25 mL) directly to reactor at default speed with stirring at 250 rpm.
11) Transfer 50 mL from reactor directly to separator at default speed, without flushing tubing after the transfer.
12) Add diethyl ether (25 mL) directly to reactor at default speed with stirring at 250 rpm.
13) Transfer 50 mL from reactor directly to separator at default speed, without flushing tubing after the transfer.
14) Extract contents of separator with diethyl ether (2×75 mL). Transfer waste phase (bottom) to waste, and product phase (top) directly to separator.
15) Wash contents of separator with 2 N hydrochloric acid (1×62 mL). Transfer waste phase (bottom) to waste, and product phase (top) directly to separator.
16) Wash contents of separator with saturated sodium bicarbonate solution (1×62 mL). Transfer waste phase (bottom) to waste, and product phase (top) directly to separator.
17) Wash contents of separator with brine (2×100 mL). Transfer waste phase (bottom) to waste, and product phase (top) through anhydrous magnesium sulfate to rotavap.
18) Evaporate contents of rotavap at 422 mbar at 50° C. for 30 min.
19) Dry contents of rotavap for 30 min at default pressure at 50° C. Stop heating when step finishes.

The crude product was recrystallized from acetone (20 mL) to give cholesta-3,5-diene SI-35 (1.30 g, 3.52 mmol, 71%) as pale beige needle-shaped crystals. The recorded NMR signals are in agreement with the literature data. (59)

### XDL Database

The XDL database enables quick and efficient querying of both validated and unvalidated XDL syntheses. Entries can be inspected and downloaded via the ChemIDE database interface (https::/croningroup.gitlab.io/ChemPU/xdlapp/) or using the PythonAPI (https:://gitlab.com/croningroup/ChemPU/chemifydb).

Example XDL Database entry schema are shown below. The base table ‘Experiments’ gathers the key information and meta data of the experiment. The meaning of the individual entries in the base tables is set out below.
Experiment Table
Experiments(
   ID varchar(255) UNIQUE NOT NULL PRIMARY KEY,
   Username varchar(255) NOT NULL,
   ExperimentCode varchar(255),
   Public boolean,
   Description text,
   SMILE text,
   INCHI text,
   Validated boolean NOT NULL,
   Success boolean,
   Keywords text,
   ExperimentSeries varchar(255),
   Iteration integer,
   ExactRepeat Boolean,
   ExactRepeatOfExperiment varchar(255),
   EstimatedDuration real,
   ExperimentDuration real,
   ReactionClass varchar(255) NOT NULL,
   Scale real,
   Datetime timestamp,
   Location varchar(255)
)

TABLE 2
Explanation of the entries of the basic table

| Entry | Comment |
|---|---|
| ID | Unique identifier |
| Username | Owner of the entry |
| Experiment code | Laboratory code (reference to lab notebook) of the experiment |
| Public | Defines whether the entry is public or private |
| Description | A short description of what is important about this reaction. |
| SMILE | SMILE of the reaction |
| INCHI | INCHI of the target product |
| Success | Defines whether the outcome of the reaction was successful or not. |
| Keywords | Keywords that are characteristic for the reaction (e.g. ‘metathesis’) |
| Experiment series | An identifier for all runs that have been performed for this reaction. |
| Iteration | The iteration of this experiment in the experiment series (e.g. the |
| | third attempt on a reaction with modified conditions would be |
| | iteration 3) |
| Exact repeat | Number of the exact repeat of an experiment |
| Exact repeat of | ID of the experiment entry that has been repeated exactly |
| experiment | |
| Estimated duration | The estimated run time of the experiment |
| Reaction class | The reaction class the experiment belongs to. The available reaction |
| | classes are listed in Table 3 |
| Scale | The amount of the limiting reagent in mol or mmol |
| Datetime | Starting date of the experiment |
| Location | Address of the lab in which the experiment was performed |

TABLE 3
List of used reaction classes

| Entry | Reaction class |
|---|---|
| 1 | C—C bond formation (TM catalysed) |
| 2 | C—C bond formation (TM non-catalysed) |
| 3 | Heteroatom alkylation and arylation |
| 4 | Functional group manipulation |
| 5 | Protecting group manipulation |
| 6 | Ring and heterocycle formation |
| 7 | Reductions and oxidation |
| 8 | Multicomponent reactions |
| 9 | Other |

The ‘XDLEntry’ table gathers the information associated with the experimental procedure and execution. The meaning of the entries in this table (Scheme 1) is defined in Table 4.
XDLEntry (
   ExperimentID varchar(255) REFERENCES Experiments (ID),
   XDLVersion real NOT NULL,
   XDL text NOT NULL,
   XDLEXE text NOT NULL,
   Graph text NOT NULL,
   SimulationLog text,
   ExecutionLog text,
   ExecutionScript text,
   LiteratureProcedure text
)

Scheme 2: The XDLEntry schema captures all entries that are directly or indirectly relevant for the actual procedure and links it to the experiment ID defined in the top-level schema.

TABLE 4
Explanation of the entries of the XDLentry table

| Entry | Comment |
|---|---|
| Experiment | ID of the database entry as defined in the base table |
| ID | (Scheme 3) |
| XDL version | Version of the XDL code the experiment has been run with |
| XDL | The XDL file for the experiment |
| XDLEXE | The XDLEXE file for the experiment |
| Graph | The graph file for the experiment |
| Simulation | The folder containing the simulation logs of the |
| log | experiment |
| Execution | The folder containing the execution logs of the |
| log | experiment |
| Execution | The Python script used to compile, simulate, and execute |
| script | the XDL. |
| Literature | The prose procedure text for the experiment (if |
| procedure | available) |

The ‘Results’ table gathers all information related to the outcome of the experiment. The meaning of the entries in this table (Scheme 3) is defined in Table 5.
Results (
   ExperimentID varchar(255) REFERENCES Experiments (ID),
   Yield varchar(64),
   Purity varchar(64),
   NMRYield varchar(64),
   LiteratureYield varchar(64),
   LiteraturePurity varchar(64)
)

The Results schema captures the entries that quantify the experiment outcome in terms of yield and purity and allows for direct comparison to previously reported yields and purities and links it to the experiment ID defined in the top-level schema.

TABLE 5
Explanation of the entries of the Results table

| Entry | Comment |
|---|---|
| Experiment | ID of the database entry as defined in the base table |
| ID | (Scheme 4) |
| Yield | The yield of the target product from this reaction |
| Purity | The purity of the target product from this reaction |
| NMR yield | The yield of the target product as determined by NMR from |
| | this reaction (if available) |
| Literature | The yield reported in the literature for the product from |
| yield | this reaction (if available) |
| Literature | The purity reported in the literature for the product from |
| purity | this reaction (if available) |

The Analysis table gathers all information related to the analysis of the product(s) form this experiment. The meaning of the entries in this table is shown below.
Analysis (
   ExperimentID varchar(255) REFERENCES Experiments (ID),
   HNMR text,
   CNMR text,
   MS text,
   SensorData text
   Misc text
)

The Analysis schema captures all analysis results and sensor feedback that have been obtained for the experiment and links it to the experiment ID defined in the top-level schema

TABLE 6
Explanation of the entries of the Analysis table.

| Entry | Comment |
|---|---|
| Experiment | ID of the database entry as defined in the base table |
| ID | (Scheme 5) |
| 1H-NMR | Folder containing the raw data from a proton NMR |
| | experiment |
| 13C-NMR | Folder containing the raw data from a carbon NMR |
| | experiment |
| MS | Mass spectrum analysis |
| Sensor | Folder containing the data from various sensors (e.g. |
| data | hotplate temperature probes, conductivity sensor, etc) |
| Misc | Folder containing any other analysis that has been |
| | performed |

### REFERENCES

A number of publications are cited above in order to more fully describe and disclose the invention and the state of the art to which the invention pertains. Full citations for these references are provided below. The entirety of each of these references is incorporated herein.
1. Warr. Mol Inform 33, 469 (2014).
2. Baker Nature 533, 452 (2016).
3. Gelernter et al., Journal of Chemical Information and Computer Sciences 30, 492 (1990).
4. Segler et al., Nature 555, 604 (2018).
5. Engkvist et al. Drug Discov Today 23, 1203 (2018).
6. Grzybowski, K. J. Bishop, B. Kowalczyk, C. E. Wilmer. Nat Chem 1, 31 (2009).
7. Davies. Nature 570, 175-181 (2019).
8. Matosin et al., Dis Model Mech 7, 171 (2014).
9. Trobe et al., Angew Chem Int Ed Engl 57, 4192 (2018).
10. Li et al. Science 347, 1221 (2015).
11. Coley et al. Science 365, (2019).
12. Jiang et al. Chem Sci 12, 6977 (2021).
13. Bedard et al. Science 361, 1220 (2018).
14. Steiner et al. Science 363, 144 (2019).
15. Mehr et al., Science 370, 101 (2020).
16. Angelone et al., Nat Chem 13, 63 (2021).
17. Nantermet. Chem 1, 335 (2016).
18. Wang et al., Drug Discov Today 25, 2006-2011 (2020).
19. Merrifield Science 150, 178 (1965).
20. Plante et al., Science 291, 1523-1527 (2001).
21. Alvaradourbina et al. Science 214, 270 (1981).
22. Legrand, P. Bolla. J Autom Chem 7, 31 (1985).
23. Boga et al. React Chem Eng 2, 446 (2017).
24. Burger et al. Nature 583, 237 (2020).
25. Godfrey et al., Drug Discov Today 18, 795 (2013).
28. Orita et al., Org Process Res Dev 4, 333 (2000).
29. Tanaka et al., Org Process Res Dev 13, 1111 (2009).
30. Chatterjee et al., Nature 579, 379 (2020).
31. Hammer et al., JACS Au 1, 1572 (2021).
32. Wilbraham et al., Accounts Chem Res 54, 253 (2021).
33. Craven et al., ChemIDE. https://croningroup.gitlab.io/chemputer/xdlapp/ (2021).
34. Roughley et al., J Med Chem 54, 3451-3479 (2011).
35. Vasilevich et al., J Med Chem 55, 7003 (2012).
36. Schneider et al., J Med Chem 59, 4385-4402 (2016).
37. Carey et al., Org Biomol Chem 4, 2337 (2006).
38. Baran et al., J Am Chem Soc 140, 4751 (2018).
39. Isidro-Llobet et al., Chem Rev 109, 2455 (2009).
40. Volbeda et al., in Protecting Groups. (2019), pp. 1-27.
41. Fernandes et al., Chem Commun (Camb) 56, 8569 (2020).
42. Roush et al., Organic Syntheses. http://www.orgsyn.org/ (2021)
43. Banerjee et al. Inorg Chem 41, 6417-6425 (2002).
U.S. Pat. No. 5,463,564
U.S. Pat. No. 7,164,992 WO 2021/219999 WO 2019/137954

The following citations are taken from the ChemPU and Synthesis Protocols section of the application:
1. Angelone et al., Nature Chemistry 13, 63-69 (2021).
2. Fyfe et al., Chemistry-a European Journal 21, 8951-8964 (2015).
3. Everson et al., Organic Synth 90, 200-214 (2013).
4. He et al. Tetrahedron Lett 56, 4558-4558 (2015).
5. Kohnen et al., Organic syntheses; an annual publication of satisfactory methods for the preparation of organic chemicals 84, 88 (2007).
6. Yamagishi et al., Org Lett 13, 4873-4875 (2011).
7. Liu et al., J Org Chem 72, 8824-8830 (2007).
8. Mohr et al., Organic Synth 86, 194-211 (2009).
9. Marziale et al., Adv Synth Catal 357, 2238-2245 (2015).
10. Zhao et al., The Journal of Organic Chemistry 56, 3001-3006 (2002).
11. Flagstad et al., Eur J Org Chem 2018, 5023-5029 (2018).
12. Kumar et al., Acs Combinatorial Science 13, 466-477 (2011).
13. Jayanth et al., Org Lett 11, 815-818 (2009).
14. Xiang et al., Org Lett 6, 3155-3158 (2004).
15. Mook et al., Org Synth 66, 75-86 (1988).
16. Nyfeler et al., Org Lett 10, 985-988 (2008).
17. Catti et al., Chem Commun 51, 892-894 (2015).
18. Burks et al., Organic Process Research & Development 1, 198-210 (1997).
19. Nocquet et al., Eur J Org Chem 2016, 1156-1164 (2016).
20. Dai et al., Journal of Flow Chemistry 5, 133-138 (2015).
21. Banerjee et al., Inorg Chem 41, 6417-6425 (2002).
22. Sagamanova et al., Organic Syntheses, Vol 84 84, 359-367 (2007).
23. Okaya et al., Org Synth 93, 63-+(2016).
24. Shen et al., Tetrahedron 68, 8916-8923 (2012).
25. Nardi et al., Rsc Adv 5, 18751-18760 (2015).
26. Doyle et al. Organic Synthesis, Vol 73 73, 13-24 (1996).
27. Jacques et al., Org Synth 67, 1-12 (1989).
28. Tamaki et al., Tetrahedron Lett 81, 153364-153367 (2021).
29. Shipilovskikh et al., Org Lett 20, 728-731 (2018).
30. Dang et al., Acs Med Chem Lett 3, 925-930 (2012).
31. Hauser et al., Synthesis-Stuttgart 50, 4490-4500 (2018).
32. Romanov-Michailidis et al., Org Lett 14, 4906-4909 (2012).
33. Pang et al., Chem Commun 54, 10324-10327 (2018).
34. Charles et al., Org Lett 7, 3965-3968 (2005).
35. Villieras et al., Org Synth 66, 220-224 (1988).
36. Talley et al., Org Synth 60, 14-17 (1981).
37. Johnston et al., J Am Chem Soc 140, 11112-11124 (2018).
38. Itami et al., Journal of the American Chemical Society 122, 12013-12014 (2000).
39. Callam T. L. Lowary, Journal of Chemical Education 78, (2001).
40. More et al., Org Lett 23, 5506-5511 (2021).
41. Cranwell et al., Journal of Chemical Education 92, 1110-1114 (2015).
42. Jin et al., J Org Chem 77, 11368-11371 (2012).
43. Bach, Org Synth 60, 63-66 (1981).
44. Limnios, J Org Chem 79, 4270-4276 (2014).
45. S Danishefsky, Org Synth 60, 66-71 (1981).
46. Rawling et al., Chemical Science 5, 1777-1785 (2014).
47. Campos, et al., Org Lett 6, 79-82 (2004).
48. Anderson, Org Synth 60, 34-40 (1981).
49. Lange et al., J Am Chem Soc 139, 2053-2059 (2017).
50. Velezheva et al., J Heterocyclic Chem 43, 873-879 (2006).
51. Gogoi et al., Synthetic Communications 35, 2811-2818 (2006).
52. Sorgi et al., Org Synth 75, 215-215 (2003).
53. Kirk et al., Org Synth 75, 215-212 (1998).

1. A method for generating a database for chemical syntheses, the method comprising the steps of:
(i) generating a series of instruction sets for a series of chemical syntheses from the literature, wherein each instruction set is a machine readable and executable universal language for a chemical synthesiser; and
(ii) assembling the series of instructions sets within a database, and making the instruction sets available for access and autonomous execution by a chemical synthesiser.

2. The method of claim 1, wherein an instruction set is generated together with analytical data for the synthesis, and wherein the instruction set is made available for access together with the analytical data for access by a chemical synthesiser.

3. The method of claim 1, wherein an instruction set is made available for access together with information for access by a chemical synthesiser, which information is selected from:
(i) a user ranking of the instruction set;
(ii) the identity of the originator of the instruction set;
(iii) the validation status of an instruction set;
(iv) the access number for the instruction set, which access number is the number of times that instructions set has been viewed or the number of times the instruction set has been provided to a chemical synthesiser for execution; and
(v) variant instruction sets for the chemical synthesis, where a variant instructions set is one in which at least one operation within the instruction set is modified over the instruction set, where a variant instruction set may give rise a reaction outcome for the chemical synthesis that differs from the reaction outcome for the chemical synthesis executed under the instruction set.

4. The method of claim 1, wherein the instruction sets are made available only to authorised chemical synthesisers.

5. The method of claim 1, further comprising performing a chemical synthesis, the chemical synthesis comprising the steps of:
(i) accessing an instruction set from the database, and providing the instruction set to a chemical synthesiser and
(ii) autonomously executing the instruction set on the chemical synthesiser thereby to perform the chemical synthesis.

6. The method of claim 5, wherein the chemical synthesiser has a reactor, a separator, an evaporator and a purification system.

7. The method of claim 6, wherein the purification system includes a chromatographic separator.

8. The method of claim 5, wherein the result of the chemical synthesis is analysed.

9. The method of claim 5, wherein the instruction set is provided to the chemical synthesiser together with analytical data, wherein the analytical data is compared with analytical data that is generated from the chemical synthesis.

10. The method of claim 5, comprising the step of (iii) modifying an operation within the instruction set, and subsequently autonomously executing the modified instruction set on the chemical synthesiser thereby to perform the chemical synthesis, and analytical data that is generated from the chemical synthesis executed under the modified instruction set is compared with the analytical data provided with the instructions.

11. The method of claim 1, further comprising the step of validating an instruction set by performing a chemical synthesis executed under the instruction set and assessing the reaction outcome against the literature reported outcome for the chemical synthesis.

12. The method of claim 3, wherein the validation status of an instruction set further comprises where validated, the identity of the validator of the instruction set.

13. The method of claim 3, wherein the access number for the instruction set further comprises a relative ranking of the instructions set amongst a group of instruction sets available from the database.

14. The method of claim 8, wherein the result of the chemical synthesis is reported to the database.

15. A method for performing a chemical synthesis, the method comprising the steps of:
(i) accessing an instruction set from a database, and providing the instruction set to a chemical synthesiser, where the instruction set is a machine readable and executable universal language for a chemical synthesiser; and
(ii) autonomously executing the instruction set on the chemical synthesiser thereby to perform the chemical synthesis.

16. The method of claim 15, wherein the database is generated by the method of claim 1.

17. The method of claim 15, wherein the result of the chemical synthesis is analysed.

18. The method of claim 17, wherein the result of the chemical synthesis is reported to the database.

19. The method of claim 15, wherein the instruction set is provided to the chemical synthesiser together with analytical data, wherein the analytical data is compared with analytical data that is generated from the chemical synthesis.

20. The method of claim 15, comprising the step of (iii) modifying an operation within the instruction set, and subsequently autonomously executing the modified instruction set on the chemical synthesiser thereby to perform the chemical synthesis, and analytical data that is generated from the chemical synthesis executed under the modified instruction set is compared with the analytical data provided with the instructions.
